SEQUENCE LISTING (29 sequences)

SEQ ID NO: 1 - DNA, 24 bp, Artificial Sequence
  Description of Artificial Sequence Synthetic primer
aacattgggccacagacctacttc

SEQ ID NO: 2 - DNA, 23 bp, Artificial Sequence
  Description of Artificial Sequence Synthetic primer
gatggagctctgggtgaggatga

SEQ ID NO: 3 - DNA, 25 bp, Artificial Sequence
  Description of Artificial Sequence Synthetic primer
cgtgccccgacccacgccagcatgg

SEQ ID NO: 4 - DNA, 25 bp, Artificial Sequence
  Description of Artificial Sequence Synthetic primer
gcctccgagggccagcgagcagagc

SEQ ID NO: 5 - DNA, 20 bp, Artificial Sequence
  Description of Artificial Sequence Synthetic primer
tacctcactcgctcggctat

SEQ ID NO: 6 - DNA, 20 bp, Artificial Sequence
  Description of Artificial Sequence Synthetic primer
gatgcagatcagcagcagac

SEQ ID NO: 7 - DNA, 20 bp, Artificial Sequence
  Description of Artificial Sequence Synthetic primer
tgagacgctcaagtttgtgg

SEQ ID NO: 8 - DNA, 20 bp, Artificial Sequence
  Description of Artificial Sequence Synthetic primer
atgcagagggctggtagatg

SEQ ID NO: 9 - DNA, 24 bp, Artificial Sequence
  Description of Artificial Sequence Synthetic primer
gcctcagaagaactggtacatcag

SEQ ID NO: 10 - DNA, 24 bp, Artificial Sequence
  Description of Artificial Sequence Synthetic primer
ggaaggtcagctggatagcgacat

SEQ ID NO: 11 - DNA, 24 bp, Artificial Sequence
  Description of Artificial Sequence Synthetic primer
ggaaacagaggtgaaagaggatct

SEQ ID NO: 12 - DNA, 24 bp, Artificial Sequence
  Description of Artificial Sequence Synthetic primer
tttcacctccaactccagcaatgg

SEQ ID NO: 13 - DNA, 24 bp, Artificial Sequence
  Description of Artificial Sequence Synthetic primer
attgaccggttgagcaaggatgag

SEQ ID NO: 14 - DNA, 24 bp, Artificial Sequence
  Description of Artificial Sequence Synthetic primer
ctcttgcatctggttgtggctgta

SEQ ID NO: 15 - DNA, 24 bp, Artificial Sequence
  Description of Artificial Sequence Synthetic primer
aacattgggccacagacctacttc

SEQ ID NO: 16 - DNA, 23 bp, Artificial Sequence
  Description of Artificial Sequence Synthetic primer
gatggagctctgggtgaggatga

SEQ ID NO: 17 - DNA, 22 bp, Artificial Sequence
  Description of Artificial Sequence Synthetic primer
atcggcagaaggggcaaagtag

SEQ ID NO: 18 - DNA, 25 bp, Artificial Sequence
  Description of Artificial Sequence Synthetic primer
gcaacgcagacttctcatcttcaag

SEQ ID NO: 19 - DNA, 22 bp, Artificial Sequence
  Description of Artificial Sequence Synthetic primer
ccttttctgacatcgctctgaa

SEQ ID NO: 20 - DNA, 20 bp, Artificial Sequence
  Description of Artificial Sequence Synthetic primer
cgagtcgtttggctgggata

SEQ ID NO: 21 - DNA, 20 bp, Artificial Sequence
  Description of Artificial Sequence Synthetic primer
aggacctgtacgccaacaac

SEQ ID NO: 22 - DNA, 20 bp, Artificial Sequence
  Description of Artificial Sequence Synthetic primer
acatctgctggaaggtggac

SEQ ID NO: 23 - protein, 326 aa, Homo sapiens
MPPSISAFQAAYIGIEVLIALVSVPGNVLVIWAVKVNQALRDATFCFIVSLAVADVAVGALVIPLAILINIGPQTYFHTCLMVACPVLILTQSSILALLAIAVDRYLRVKIPLRYKMVVTPRRAAVAIAGCWILSFVVGLTPMFGWNNLSAVERAWAANGSMGEPVIKCEFEKVISMEYMVYFNFFVWVLPPLLLMVLIYLEVFYLIRKQLNKKVSASSGDPQKYYGKELKIAKSLALILFLFALSWLPLHILNCITLFCPSCHKPSILTYIAIFLTHGNSAMNPIVYAFRIQKFRVTFLKIWNDHFRCQPAPPIDEDLPEERPDD

SEQ ID NO: 24 - protein, 326 aa, Mus sp.
MPPYISAFQAAYIGIEVLIALVSVPGNVLVIWAVKVNQALRDATFCFIVSLAVADVAVGALVIPLAILINIGPQTYFHTCLMVACPVLILTQSSILALLAIAVDRYLRVKIPLRYKTVVTQRRAAVAIAGCWILSLVVGLTPMFGWNNLSEVEQAWIANGSVGEPVIKCEFEKVISMEYMVYFNFFVWVLPPLLLMVLIYLEVFYLIRKQLNKKVSASSGDPQKYYGKELKIAKSLALILFLFALSWLPLHILNCITLFCPTCQKPSILIYIAIFLTHGNSAMNPIVYAFRIHKFRVTFLKIWNDHFRCQPKPPIEEDIPEEKADD

SEQ ID NO: 25 - DNA, 2937 bp, Homo sapiens
gtgcggagcccgattgtcactcagctcctgcgccgggggcgacagagccgcaggcgcccgagtcgagtcccagccagctaccatccctctggagcttaccggccggccttggcttccccaggaatccctggagctagcggctgctgaaggcgtcgaggtgtgggggcacttggacagaacagtcaggcagccgggagctctgccagctttggtgaccttgggccgggctgggagcgctgcggcgggagccggaggactatgagctgccgcgcgttgtccagagcccagcccagccctaccgcgcgcggcccggagctctgttccctggaactttgggcactgcctctgggacccctgccggccagcaggcaggatggtgcttgcctcgtgccccttggtgcccgtctgctgatgtgcccagcctgtgcccgccatgccgccctccatctcagctttccaggccgcctacatcggcatcgaggtgctcatcgccctggtctctgtgcccgggaacgtgctggtgatctgggcggtgaaggtgaaccaggcgctgcgggatgccaccttctgcttcatcgtgtcgctggcggtggctgatgtggccgtgggtgccctggtcatccccctcgccatcctcatcaacattgggccacagacctacttccacacctgcctcatggttgcctgtccggtcctcatcctcacccagagctccatcctggccctgctggcaattgctgtggaccgctacctccgggtcaagatccctctccggtacaagatggtggtgaccccccggagggcggcggtggccatagccggctgctggatcctctccttcgtggtgggactgacccctatgtttggctggaacaatctgagtgcggtggagcgggcctgggcagccaacggcagcatgggggagcccgtgatcaagtgcgagttcgagaaggtcatcagcatggagtacatggtctacttcaacttctttgtgtgggtgctgcccccgcttctcctcatggtcctcatctacctggaggtcttctacctaatccgcaagcagctcaacaagaaggtgtcggcctcctccggcgacccgcagaagtactatgggaaggagctgaagatcgccaagtcgctggccctcatcctcttcctctttgccctcagctggctgcctttgcacatcctcaactgcatcaccctcttctgcccgtcctgccacaagcccagcatccttacctacattgccatcttcctcacgcacggcaactcggccatgaaccccattgtctatgccttccgcatccagaagttccgcgtcaccttccttaagatttggaatgaccatttccgctgccagcctgcacctcccattgacgaggatctcccagaagagaggcctgatgactagaccccgccttccgctcccaccagcccacatccagtggggtctcagtccagtcctcacatgcccgctgtcccaggggtctccctgagcctgccccagctgggctgttggctgggggcatgggggaggctctgaagagatacccacagagtgtggtccctccactaggagttaactaccctacacctctgggccctgcaggaggcctgggagggcaagggtcctacggagggaccaggtgtctagaggcaacagtgttctgagcccccacctgcctgaccatcccatgagcagtccagagcttcagggctgggcaggtcctggggaggctgagactgcagaggagccacctgggctgggagaaggtgcttgggcttctgcggtgaggcaggggagtctgcttgtcttagatgttggtggtgcagccccaggaccaagcttaaggagaggagagcatctgctctgagacggatggaaggagagaggttgaggatgcactggcctgttctgtaggagagactggccagaggcagctaaggggcaggaatcaaggagcctccgttcccacctctgaggactctggaccccaggccataccaggtgctagggtgcctgctctccttgccctgggccagcccaggattgtacgtgggagaggcagaaagggtaggttcagtaatcatttctgatatttgctggagtgctggctccacgccctggggagtgagcttggtgcggtaggtgctggcctcaaacagccacgaggtggtagctctgagccctccttcttgccctgagctttccggggaggagccttggagtgtaattacctgtcatctgggccaccagctccactggcctgcccgttgccgggcctggactgtcctaggtgaccccatctctgctgcttctgggcctgatggagaggagaacactagacatgccaactcgggagcattctgcctgcctgggaacggggtggacgagggagtgtctgtaaggactcagtgttgactgtaggcgcccctggggtgggtttagcaggctgcagcaggcagaggagagtacccccctgagagcatgtgggggaaggccttgctgtcatgtgaatccctcaatacccctagtatctggctgggttttcaggggctttggaagctctgttgcaggtgtccgggggtctaggactttagggatctggggaaggaccaacccatgccctgccaagcctggagcccctgtgttggggggcaaggtgggggagcctggagcccctgtgtgggagggcgaggcgggggagcctggagcccctgtgtgggagggcgaggcgggggatcctggagcccctgtgtcggggggcgagggaggggaggtggccgtcgagttgaccttctgaacatgagtgtcaactccaggacttgcttccaagcccttccctctgttggaaattgggtgtgccctggctcccaagggaggcccatgtgactaataaaaaactgtgaaccctgaaaaaaaaaaaaaaaaaa

SEQ ID NO: 26 - protein, 326 aa, Homo sapiens; identical to SEQ ID NO: 23

SEQ ID NO: 27 - DNA, 2590 bp, Homo sapiens
acagcctgtgtacatgtgagcagccgcgtgcagaggggaagctctgcctgggcctcagggactgtgacatggagcaggagccgcccccagccaagctgctttcagcacagcgtgggcccccagcaccttggtgcggggtgcggcccctcggaggagggctgtcaggtgaagcctcgtgtgagggggtgcctcaggaaccctgaagctgggctgagccatgatgctgctgccagaacccctgcagagggcctggtttcaggagactcagagtcctctgtgaaaaagcccttggagagcgccccagcagggctgcacttggctcctgtgaggaaggggctcaggggtctgggcccctccgcctgggccgggctgggagccaggcgggcggctgggctgcagcaatggaccgtgagctggcccagcccgcgtccgtgctgagcctgcctgtcgtctgtggccatgcccatcatgggctcctcggtgtacatcacggtggagctggccattgctgtgctggccatcctgggcaatgtgctggtgtgctgggccgtgtggctcaacagcaacctgcagaacgtcaccaactactttgtggtgtcactggcggcggccgacatcgcagtgggtgtgctcgccatcccctttgccatcaccatcagcaccgggttctgcgctgcctgccacggctgcctcttcattgcctgcttcgtcctggtcctcacgcagagctccatcttcagtctcctggccatcgccattgaccgctacattgccatccgcatcccgctccggtacaatggcttggtgaccggcacgagggctaagggcatcattgccatctgctgggtgctgtcgtttgccatcggcctgactcccatgctaggttggaacaactgcggtcagccaaaggagggcaagaaccactcccagggctgcggggagggccaagtggcctgtctctttgaggatgtggtccccatgaactacatggtgtacttcaacttctttgcctgtgtgctggtgcccctgctgctcatgctgggtgtctatttgcggatcttcctggcggcgcgacgacagctgaagcagatggagagccagcctctgccgggggagcgggcacggtccacactgcagaaggaggtccatgctgccaagtcactggccatcattgtggggctctttgccctctgctggctgcccctacacatcatcaactgcttcactttcttctgccccgactgcagccacgcccctctctggctcatgtacctggccatcgtcctctcccacaccaattcggttgtgaatcccttcatctacgcctaccgtatccgcgagttccgccagaccttccgcaagatcattcgcagccacgtcctgaggcagcaagaacctttcaaggcagctggcaccagtgcccgggtcttggcagctcatggcagtgacggagagcaggtcagcctccgtctcaacggccacccgccaggagtgtgggccaacggcagtgctccccaccctgagcggaggcccaatggctatgccctggggctggtgagtggagggagtgcccaagagtcccaggggaacacgggcctcccagacgtggagctccttagccatgagctcaagggagtgtgcccagagccccctggcctagatgaccccctggcccaggatggagcaggagtgtcctgatgattcatggagtttgccccttcctaagggaaggagatctttatctttctggttggcttgaccagtcacgttgggagaagagagagagtgccaggagaccctgagggcagccggttcctactttggactgagagaagggagccccaggctggagcagcatgaggcccagcaagaagggcttgggttctgaggaagcagatgtttcatgctgtgaggccttgcaccaggtgggggccacagcaccagcagcatctttgctgggcagggcccagccctccactgcagaagcatctggaagcaccaccttgtctccacagagcagcttgggcacagcagactggcctggccctgagactggggagtggctccaacagcctcctgccacccacacaccactctccctagactctcctagggttcaggagctgctgggcccagaggtgacatttgacttttttccaggaaaaatgtaagtgtgaggaaaccctttttattttattacctttcactctctggctgctgggtctgccgtcggtcctgctgctaacctggcaccagagcctctgcccggggagcctcaggcagtcctctcctgctgtcacagctgccatccacttctcagtcccagggccatctcttggagtgacaaagctgggatcaaggacagggagttgtaacagagcagtgccagagcatgggcccaggtcccaggggagaggttggggctggcaggccactggcatgtgctgagtagcgcagagctacccagtgagaggccttgtctaactgcctttccttctaaagggaatgtttttttctgagataaaataaaaacgagccacatcgtgttttaagcttgtccaaatgaaaaaaaaaaaaaaaaaaaaaaaaa

SEQ ID NO: 28 - protein, 412 aa, Homo sapiens
MPIMGSSVYITVELAIAVLAILGNVLVCWAVWLNSNLQNVTNYFVVSLAAADIAVGVLAIPFAITISTGFCAACHGCLFIACFVLVLTQSSIFSLLAIAIDRYIAIRIPLRYNGLVTGTRAKGIIAICWVLSFAIGLTPMLGWNNCGQPKEGKNHSQGCGEGQVACLFEDVVPMNYMVYFNFFACVLVPLLLMLGVYLRIFLAARRQLKQMESQPLPGERARSTLQKEVHAAKSLAIIVGLFALCWLPLHIINCFTFFCPDCSHAPLWLMYLAIVLSHTNSVVNPFIYAYRIREFRQTFRKIIRSHVLRQQEPFKAAGTSARVLAAHGSDGEQVSLRLNGHPPGVWANGSAPHPERRPNGYALGLVSGGSAQESQGNTGLPDVELLSHELKGVCPEPPGLDDPLAQDGAGVS

SEQ ID NO: 29 - DNA, 24 bp, Artificial Sequence
  Description of Artificial Sequence Synthetic primer
tttttttttttttttttttttttt

(19) United States
(12) Patent Application Publication
(10) Pub. No.: US 2010/0272711 A1
(43) Pub. Date: Oct. 28, 2010

(54) COMPOSITIONS AND METHODS FOR THE TREATMENT AND PREVENTION OF CARDIOVASCULAR DISEASES

(75) Inventors: Arthur Feldman, Wynnewood, PA (US); Tung Chan, Huntington Valley, PA (US)

Correspondence Address: DAVID S. RESNICK, NIXON PEABODY LLP, 100 SUMMER STREET, BOSTON, MA, 02110-2131, US

(73) Assignee: THOMAS JEFFERSON UNIVERSITY, PHILADELPHIA, PA (US)

(21) Appl. No.: 12/747,147

(22) PCT Filed: Dec. 12, 2008
(86) PCT No.: PCT/US08/86528

§ 371 Date: Jul. 6, 2010

Related U.S. Application Data
(60) Provisional application No. 61/013,057, filed on Dec. 12, 2007.

(51) Int. Cl. A61K 39/395; A61K 38/14; A61K 31/7088; A61K 31/7084; A61K 31/7052; A61K 38/00; A61K 31/437; A61K 31/7076; A61K 31/538; A61K 31/502; A61P 9/04; A61P 9/00; A61P 9/10

(52) U.S. Cl. 424/130.1; 514/44 R; 514/20.9; 514/1.1; 514/44 A; 514/303; 514/46; 514/230.5; 514/248

(57) ABSTRACT

The present invention is directed to a pharmaceutical composition, and methods of use thereof, comprising at least one agent which target multiple adenosine receptors (AR) simultaneously in a stoichiometric relationship (i.e. each AR receptor is targeted to an equal extent). Aspects of the present invention relate to pharmaceutical compositions, and uses thereof, comprising at least one agent which co-activates an A₁-adenosine receptor (A₁-AR) and an A_{2A}-adenosine receptor (A_{2A}-AR) or a combination of at least one agent which activates an A₁-AR and at least one agent which activates an A_{2A}-AR, where both the A₁-AR and A_{2A}-AR are activated in a stoichiometric relationship such that the level of biological activation of A₁-AR is approximately the same level of biological activation of A_{2A}-AR. Other aspects of the present invention relates to methods for the therapeutic and prophylactic treatment of cardiac dysfunction in a subject having or at risk of having a cardiac dysfunction, for example, but not limited to, for the treatment of a subject with myocardial infarction, such as acute myocardial infarction, coronary ischemia or congestive heart failure and other cardiac dysfunctions.

### CROSS REFERENCE TO RELATED APPLICATIONS

This application claims the benefit under 35 U.S.C. 119(e) of U.S. Provisional Patent Application Ser. No. 61/013,057 filed Dec. 12, 2007, the contents of which are incorporated herein by reference in their entirety.

### GOVERNMENT SUPPORT

This invention was made, in part, with Government Support under Grant Nos. HL61690, HL075443 and R01 DK68575 awarded by National Institutes of Health (NIH). The government of the United States has certain rights to the invention.

### FIELD OF THE INVENTION

The present invention relates generally to methods to activate A1 adenosine (A₁-AR) and A2A adenosine receptor (A_{2A}-AR) simultaneously for the treatment and/or prevention of cardiac dysfunction, for example myocardial infarction.

### BACKGROUND OF THE INVENTION

Adenosine is an endogenous purine nucleoside that plays an important role in protecting the heart during stress. In animal models of ischemia, adenosine reduces infarct size,^{1,2} affords protection from re-perfusion injury after prolonged coronary occlusion,³ and facilitates ischemic preconditioning.⁴ Furthermore, adenosine infusion reduced infarct size in patients with a myocardial infarction.⁵ Because of its pharmacologic effects on neurohormone and cytokine activation,⁶⁻¹⁰ it was hypothesized that adenosine might also effect ventricular remodeling in models of heart failure. Indeed, adenosine reduced cardiac hypertrophy and improved left ventricular function in mice with transverse aortic constriction.¹¹ In addition, patients with increased muscle adenosine levels due to mutation in at least one allele of the adenosine monophosphate deaminase 1 (AMPD1) gene had a longer survival when compared to patients with the wild-type genoptype.¹²⁻¹⁴ Furthermore, in patients with heart failure, increased levels of adenosine were associated with the severity of disease.¹⁵ These initial studies led investigators to identify the specific adenosine receptor (AR) subtypes that mediated the salutary benefits of adenosine.

The cardiovascular effects of adenosine are modulated by four known G protein-coupled AR (A₁, A_{2A}, A_{2B} and A₃); all of which are expressed in the heart. Activation of the A₁- and A₃-ARs inhibits adenylyl cyclase and modulates other signaling pathways regulated by G_{i/o}. By contrast, activation of A₂ₐ-ARs couple to Gs proteins and activate adenylyl cyclase.¹⁶⁻¹⁸ Pharmacologic studies using receptor-subtype-selective agonists suggested that A₁- and A₃-ARs provide cardioprotection during ischemia and reperfusion^{19,20} while A₂-ARs afford protection post-ischemia.²¹⁻²³ Because pharmacologic agonists lack selectivity and because of significant species differences in the pharmacology of adenosine receptors, 16 recent studies have assessed the role of AR-subtypes using selective gene deletion and cardiac-restricted transgenic overexpression.^{24-26,27} Although both A₁- and A₃-ARs mediate increased myocardial resistance to ischemia, mice overexpressing modest levels of the A₁-AR demonstrated a reduction in the response to high doses of catecholamines, and an increase in hypertrophy²⁸ while mice with higher levels of A₁-AR overexpression did not tolerate myocardial ischemia and had significant atrial arrhythmias.²⁹ Of greater concern was the finding that mice with moderate to high levels of A₃-AR overexpression developed a dilated cardiomyopathy.³⁰ These findings raised concerns about the safety of chronic adenosine-AR activation in patients with cardiovascular disease.

The purine adenosine is expressed ubiquitously in mammalian tissues and facilitates a large number of intracellular processes. In the heart, adenosine has been shown to mediate the process of ischemic pre-conditioning. In ischemic pre-conditioning, brief periods of coronary occlusion (i.e. brief periods of cardiac ischemia) act to condition the heart such that a subsequent complete occlusion of the coronary results in a marked decrease in the amount of myocardial damage when compared with hearts that did not undergo pre-ischemic conditioning. In addition, adenosine is a potent inhibitor of tumor necrosis factor alpha (TNFα), a protein that is not expressed in the normal heart but which is expressed in hearts with dilated cardiomyopathy. This protein facilitates the progression of heart failure as its over-expression results in extracellular matrix remodeling through activation of matrix metalloproteinases, diminished cardiac contractility through altered regulation of calcium homeostasis, activation of programmed cell death (apoptosis), abnormal mitochondrial function and mitochondrial damage, abnormal ion channel signaling, marked cardiac dilatation and early death.

Adenosine regulates cardiac homeostasis through interacting with three distinct adenosine receptors that can be found on the surface of cardiac cells: A₁, A_{2A}, and A₃. The A₁ and A_{2A} adenosine receptors are abundant while the A₃ adenosine receptor (A₃-AR) is found in relatively small quantities. All of the adenosine receptors couple with G signal transduction proteins; A₁ and A₃ adenosine receptors couple with the G inhibitory protein (Gᵢ) while the A_{2A} adenosine receptor couples with the G stimulatory (Gₛ) protein. Identifying which receptor couples with which intracellular process has been difficult, with important information obtained from the creation of transgenic mice in which a selective receptor subtype was either over-expressed or “knocked-out.” These studies have demonstrated that the A₁ and A₃-adenosine receptors function in ischemic pre-conditioning. These studies were limited to some extent by the fact that the adenosine receptor was over-expressed or knocked-out during both fetal and prenatal development as well as during adulthood. Since adenosine has been shown to play an important role in the development of the normal heart, traditional over-expression techniques could mask or exacerbate the effects of receptor-subtype over-expression or ablation.

Both the A₁- and A₃-adenosine receptors (AR) have been implicated in mediating the cardioprotective effects of adenosine, however, overexpression of either A₁-AR alone or A₃-AR alone is associated with unfavorable changes in the cardiac phenotype.

### SUMMARY OF THE INVENTION

The inventors have discovered that continual or chronic selective overexpression of A₁-adenosine receptors (A₁-AR) compromised cardiac function. The inventors have also discovered that long term or chronic overexpression of A_{2A} adenosine receptors (A_{2A}-AR), while it leads to an initial increase in heart muscle contractibility, overtime leads to compromised cardiac function such as congestive heart failure and decreased heart rate. Therefore, the inventors have discovered that long term or chronic administration of agonists which activate only the A₁-AR or alternatively only the A_{2A}-AR results in deleterious effects on cardiac function.

Agonists which activate the A₁-AR and/or the and A₃-AR, which function to signal through the G inhibitory protein (Gᵢ) are commonly known and used by persons of ordinary skill in the art to mediate or mimic the cardioprotective effects of adenosine during ischemia and reperfusion. By contrast, agonists which activate the A_{2A}-AR results in signalling via the G stimulatory (Gs) protein to activate adenyl cyclase and afford protection post-ischemia.

As discussed herein, however, the inventors discovered that long term or chronic administration of agonists which activates only the A₁-AR results in deleterious effects on cardiac function. Similarly, the inventors discovered that long term or chronic administration of agonists which activate only the A_{2A}-AR also results in deleterious effects on cardiac function.

The inventors surprisingly also discovered that the cardioprotective phenotype was restored by simultaneous and equal activation and/or overexpression of the A₁- and the A_{2A}-adenosine receptors as compared to single receptor subtype activation. Thus, the inventors discovered that if both the A₁-AR and the A_{2A}-AR are co-activated substantially simultaneously, the cardiac function was unexpectedly not compromised as compared to single receptor activation of A₁-AR or the A_{2A}-AR. This is surprisingly due to the fact that (i) both chronic activation of either A₁-AR or A_{2A}-AR by themselves (by administration of agonists or by other means, such as induced or overexpression) results in deleterious effects on cardiac function, and (ii) combined with the fact that A₁-AR and A_{2A}-AR signal through directly opposite pathways (i.e. A₁-AR signals via Gᵢ, and A_{2A}-AR signals via Gₛ). Thus one would not expect that, due to each having deleterious effects when chronically activated by themselves and functioning via directly opposite pathways, the co-activation of both A₁-AR and the A_{2A}-AR substantially and simultaneously would be beneficial or useful for cardiac protection. Thus, the inventors have surprisingly discovered that use of at least one agent which co-activates both the A₁-AR or the A_{2A}-AR, or a combination of at least one or more agents which activates the A₁-AR and at least one or more agents which activate the A_{2A}-AR is useful to mediate cardioprotective effect.

Thus the inventors have discovered that if both the A₁- and A_{2A}-adenosine receptors are activated or overexpressed simultaneously and equally, cardiac function was not compromised as compared to single receptor A₁-AR or the A_{2A}-AR activation or overexpression. The inventors also discovered that the cardioprotective phenotype was restored on simultaneous and equal activation and/or overexpression of A₁- and A_{2A}-adenosine receptors as compared to single receptor subtype activation.

One aspect of the present invention relates to methods to treat a subject, preferably a human subject with compromised cardiac function with a pharmaceutical composition which targets multiple adenosine receptors (AR) simultaneously in a stoichiometric relationship (i.e. each AR receptor is targeted to an equal extent). In particular, in one embodiment, the present invention relates to pharmaceutical compositions comprising at least one agent or a combination of two or more agents which activate the A₁-AR and also activate the A_{2A}-AR in a stoichiometric relationship. Stated another way, one embodiment relates to a pharmaceutical composition comprising an agent or agents which can activate both the A₁-AR and A_{2A}-AR, where the level of biological activation of A₁-AR is approximately the same level of biological activation of A_{2A}-AR.

In another embodiment, the present invention relates to administration of a pharmaceutical composition comprising at least one agent which activates A₁-AR and also activates the A_{2A}-AR in a stoichiometric relationship to a subject for the treatment for cardiac dysfunction, for example, but not limited to, for the treatment of a subject with myocardial infarction, such as acute myocardial infarction, coronary ischemia, or congestive heart failure and for the treatment of subjects undergoing percutaneous coronary intervention. In one embodiment, one first determines that the individual is suffering from a chronic heart failure, for example, a subject has a myocardial infarction, or chronic or acute myocardial ischemia, cardiomyopathy, myocarditis, or other such cardiac dysfunction diseases, and then one administered said pharmaceutical compositions to the subject.

Another aspect of the present invention provides methods to screen for an agent which functions as a co-agonist or co-antagonists for the A₁-AR and A_{2A}-adenosine receptors, and in particular agonists which are stoichiometrically balanced agonists of A₁-AR and A_{2A}-AR. In one embodiment of this aspect as discussed herein, the present invention provides methods to screen for an agent which functions as a co-agonist for the A₁-AR and A_{2A}-adenosine receptors which are stoichiometrically balanced agonists of A₁-AR and A_{2A}-AR.

In another aspect of the invention, the inventors have discovered that adenosine and A₁ adenosine receptors contribute to pathobiology of heart muscle dysfunction in murine models of heart failure and chronic heart failure. The present invention therefore relates to adenosine therapeutics, such as adenosine and adenosine receptor agonists and antagonists, in particular agonists and/or antagonists targeting A₁ and A_{2A}-adenosine receptors. Murine models are well known as models for human conditions for heart failure and chronic heart failure and other cardiac disorders.

One aspect of the present invention provides methods for treating or preventing a subject with or at risk of having cardiac dysfunction comprising administering to the subject a pharmaceutical composition comprising an effective amount of at least one agent which co-activates both the A₁-adenosine receptor and activates the A_{2A}-adenosine receptor, or a combination of at least one agent which activates the A₁-adenosine receptor and at least one agent which activates the A_{2A}-adenosine receptor, wherein the pharmaceutical composition results in a level of biological activation of A1-adenosine receptors within about 10% of the level of biological activation of A_{2A}-adenosine receptors, wherein the level of A₁-adenosine receptors biological activation is measured by detecting the activation of Gi-protein, and the level of the A2_{A}-adenosine receptors is measured by detecting the activation of Gs-protein.

Another aspect of the present invention provides methods for treating or preventing cardiac dysfunction in a subject having, or at risk of having cardiac dysfunction, the method comprising administering to the subject a pharmaceutical composition comprising, or alternatively consisting essentially of, or alternatively consisting of, an effective amount of at least one agent which co-activates both an A₁-adenosine receptor (A₁-AR) and an A_{2A}-adenosine receptor (A_{2A}-AR), or a combination of at least one agent which activates an A₁-adenosine receptor (A₁-AR) and at least one agent which activates an A_{2A}-adenosine receptor (A_{2A}-AR), wherein the pharmaceutical composition results in a level of biological activation of the A₁-adenosine receptor is within about 10% of the level of biological activation of the A_{2A}-adenosine receptor, wherein the level of the A₁-adenosine receptor biological activation is measured by detecting activation of Gi-protein, and the level of the A_{2A}-adenosine receptor is measured by detecting activation of Gₛ-protein.

Another aspect of the present invention provides methods for treating or preventing cardiac dysfunction in a subject having, or at risk of having cardiac dysfunction, the method comprising administering to the subject a pharmaceutical composition comprising, or alternatively consisting essentially of, or alternatively consisting of, an effective amount of at least one agent which co-activates both an A₁-adenosine receptor (A₁-AR) and an A_{2A}-adenosine receptor (A_{2A}-AR), or a combination of at least one agent which activates an A₁-adenosine receptor (A₁-AR) and at least one agent which activates an A_{2A}-adenosine receptor (A_{2A}-AR), wherein the at least one agent that co-activates the A₁-adenosine receptor and the A_{2A}-adenosine receptors, or the at least one agent that activates the A₁-adenosine receptor has a lower Kᵢ as compared to Kᵢ of at least one agent which activates the A_{2A}-adenosine receptor.

Another aspect of the present invention provides methods for treating or preventing a subject having or at risk of having cardiac dysfunction comprising administering to the subject a pharmaceutical composition comprising, or alternatively consisting essentially of, or alternatively consisting of, an effective amount of a combination of at least one agent which activates an A₁-adenosine receptor (A₁-AR) and at least one agent which activates an A_{2A}-adenosine receptor (A_{2A}-AR), wherein the pharmaceutical composition comprises at least a 1.5 fold higher amount of the at least one agent which activates the A₁-adenosine receptor as compared to the amount of the at least one agent which activates the A_{2A}-adenosine receptor activation.

Another aspect of the present invention provides methods for enhancing cardiac function in a subject comprising; (a) selecting a subject in need of, or currently being treated an adenosine agonist therapy; (b) administering to the subject a pharmaceutical composition comprising, or alternatively consisting essentially of, or alternatively consisting of, at least one agent which co-activates both an A₁-adenosine receptor (A₁-AR) and an A_{2A}-adenosine receptor (A_{2A}-AR), or a combination of at least one agent which activates an A₁-adenosine receptor (A₁-AR) and at least one agent which activates an A_{2A}-adenosine receptor (A_{2A}-AR), wherein the level of activation of A₁-AR is about the same as the level of activation of A_{2A}-AR.

In some embodiments of all aspects of the present invention, a subject is first diagnosed (i.e. screened) for having, or at risk of having a cardiac dysfunction, and if a subject is identified to have, or be at risk of having a cardiac dysfunction, then the subject can be treated for cardiac dysfunction according to the methods as discussed herein.

Another aspect of the present invention provides methods for treating or preventing a subject with or at risk of having cardiac dysfunction comprising administering to the subject a pharmaceutical composition comprising an effective amount of at least one agent which co-activates both the A₁-adenosine receptor and activates the A_{2A}-adenosine receptor, or a combination of at least one agent which activates the A₁-adenosine receptor and at least one agent which activates the A_{2A}-adenosine receptor, wherein an that agent that activates A₁-adenosine receptor has a lower Ki as compared to Ki of the agent for the A_{2A}-adenosine receptor.

Another aspect of the present invention provides methods for treating or preventing a subject with or at risk of having cardiac dysfunction comprising administering to the subject a pharmaceutical composition comprising an effective amount of at least one agent which co-activates both the A₁-adenosine receptor and activates the A_{2A}-adenosine receptor, or a combination of at least one agent which activates both the A₁-adenosine receptor and at least one agent which activates the A_{2A}-adenosine receptor, wherein the pharmaceutical composition comprises at least about a 1.5 fold higher amount of the agent which activates the A₁-adenosine receptor as compared to the amount of the agent which activates the A_{2A}-adenosine receptor activation.

Another aspect of the present invention provides methods for enhancing cardiac function in a subject comprising; (a) selecting a subject in need of, or currently being administered an adenosine agonist therapy; (b) administering to the subject a pharmaceutical composition comprising of at least one agent which co-activates both the A₁-adenosine receptor and activates the A_{2A}-adenosine receptor, or a combination of at least one agent which activates the A₁-adenosine receptor and at least one agent which activates the A_{2A}-adenosine receptor, wherein the level of activation of A₁-AR is about the same as the level of activation of A_{2A}-AR.

Another aspect of the present invention provides methods for treating or preventing cardiac dysfunction in a subject with, or at risk of having cardiac dysfunction comprising, first diagnosing a subject with, or at risk of having a cardiac dysfunction, wherein if a subject is diagnosed with, or at risk of having a cardiac dysfunction, the subject is administered a pharmaceutical composition comprising an effective amount of at least one agent which co-activates both the A₁-adenosine receptor and activates the A_{2A}-adenosine receptor, or a combination of at least one agent which activates the A₁-adenosine receptor and at least one agent which activates the A_{2A}-adenosine receptor, wherein an that agent that activates A₁-adenosine receptor has a lower Ki as compared to Ki of the agent for the A_{2A}-adenosine receptor.

Another aspect of the present invention provides methods for treating or preventing a cardiac dysfunction in a subject with or at risk of having cardiac dysfunction comprising first diagnosing a subject with, or at risk of having a cardiac dysfunction, wherein if a subject is diagnosed with, or at risk of having a cardiac dysfunction, the subject is administered a pharmaceutical composition comprising an effective amount o of at least one agent which co-activates both the A₁-adenosine receptor and activates the A_{2A}-adenosine receptor, or a combination of at least one agent which activates the A₁-adenosine receptor and at least one agent which activates the A_{2A}-adenosine receptor, wherein the pharmaceutical composition comprises at least about a 1.5 fold higher amount of the agent which activates the A₁-adenosine receptor as compared to the amount of the agent which activates the A_{2A}-adenosine receptor activation.

Another aspect of the present invention provides methods for enhancing cardiac function in a subject comprising; (a) diagnosing a subject with, or at risk of having a cardiac dysfunction, wherein if a subject is diagnosed with, or at risk of having a cardiac dysfunction, (b) selecting the subject; (c) administering to the subject a pharmaceutical composition comprising of at least one agent which co-activates both the A₁-adenosine receptor and activates the A_{2A}-adenosine receptor, or a combination of at least one agent which activates the A₁-adenosine receptor and at least one agent which activates the A_{2A}-adenosine receptor, wherein the level of activation of A₁-AR is about the same as the level of activation of A_{2A}-AR.

In some embodiments of all aspects described herein, the pharmaceutical composition is free of a sodium-hydrogen exchanger inhibitory compound.

In some embodiments of all aspects described herein, the subject in need is at risk of having or has had myocardial infarction, for example, the subject has, or is at risk of chronic heart failure. In some embodiments of all aspects described herein, where a subject with chronic heart failure has, for example, a chronic or acute myocardial ischemia and reperfusion injury, cardiomyopathy, myocarditis, cardiac hypertrophy, ventricular remodeling, coronary ischemia or congestive heart failure. In some embodiments, a subject is undergoing coronary intervention, such as percutaneous coronary intervention. In some embodiments, the subject is prior to or undergoing or post surgery having a potential to cause cardiac ischemic damage. Alternatively, the subject can be prior to, or undergoing or post surgery which is cardiac surgery.

In some embodiments of all aspects described herein, the subject is a human subject.

In some embodiments of all aspects described herein, an agent for use in the methods and compositions as disclosed herein can be selected from the group comprising a small molecule, nucleic acid, such as siRNA, shRNA, miRNA, a nucleic acid analogue such as PNA, pc-PNA, LNA, an aptamer, a ribosome, a peptide, a protein, an avimer, an antibody, or variants and fragments thereof. In some embodiments of all aspects described herein, the agent which co-activates both the A₁-adenosine receptor and activates the A_{2A}-adenosine receptor can be AMP579 or a derivative thereof. In some embodiments of all aspects described herein, an agent can be a binary conjugate of at least one agent which activates A₁-AR and at least one agent which activates A_{2A}-AR.

Another aspect of the present invention relates to a pharmaceutical composition comprising an effective amount of at least one agent which activates the A₁-adenosine receptor and an effective amount of at least one agent which activates the A_{2A}-adenosine receptor, wherein the level of activation of A₁-AR is about the same as the level of activation of A_{2A}-AR.

In this aspect and all other aspects described herein, the pharmaceutical composition can comprise an agent which activates A₁-AR, such as, but not limited to: AB-MECA, CPA, ADAC, CCPA, CHA, GR79236, S-ENBA, IAB-MECA, R-PIA, ATL146e, CGS-21680, CV1808, NECA, PAPA-APEC, DITC APEC, DPMA, S-HPNECA, WRC-0470, IB-MECA, 2-CIADO, I-ABA, S-PIA, C1-IB MECA, polyadenylic acid, or analogues or derivatives thereof.

In this aspect and all other aspects described herein, the pharmaceutical composition can comprise an agent which activates A_{2A}-AR, such as, but is not limited to: 2-cyclohexylmethylenehydrazinoadenosine, 2-(3-cyclohexenyl)methylenehydrazinoadenosine, 2-isopropylmethylenehydrazinoadenosine, N-ethyl-1′-deoxy-1′-[6-amino-2-[(2-thiazolyl)ethynyl]-9H-purin-9-yl]-β-D-ribofuranuronamide, N-ethyl-1′-deoxy-1′-[6-amino-2-[hexynyl]-9H-purin-9-yl]-β-D-ribofuranuronamide, 2-(1-hexyn-1-yl)adenosine 5′-N-methyluronamide, 5′-chloro-5′-deoxy-2-(1-hexyn-1-yl)adenosine, N6-[2-(3,5-dimethoxyphenyl)-2-(2-methylphenyl)adenosine, 2-(2-phenyl)ethoxyadenosine, 2-[2-(4-methylphenyl)ethoxy]adenosine, 2-[2-(4-fluorophenyl)ethoxy]adenosine, 2-(2-(2-naphthyl)ethoxy)adenosine, 2-[p-(2-carboxyethyl)phenethylamino-5′-N-ethylcarboxamidoadenosine (CGS-21680), 2-(2-cyclohexyl)ethoxyadenosine, 2-octynyladenosine (YT-146), 2-thiazolylethynyladenosine and 2-phenethylamino-5′-N-ethylcarboxamidoadenosine (CGS-21577) or analogues or derivatives thereof.

In one embodiment of this aspect and all other aspects described herein, the pharmaceutical composition can comprise at least one agent which activates the A₁-adenosine receptor which is conjugated to an agent which activates the A_{2A}-adenosine receptor.

Another aspect of the present invention relates to the use of the pharmaceutical composition as described herein for the treatment or prevention of myocardial infarction in a subject, and/or for the treatment or prevention of chronic heart failure in a subject.

Another aspect of the present invention relates to the use of the pharmaceutical composition as described herein for the treatment or prevention of chronic or acute myocardial ischemia and reperfusion injury, cardiomyopathy, myocarditis, cardiac hypertrophy, ventricular remodeling, coronary ischemia or congestive heart failure in a subject.

Another aspect of the present invention is a pharmaceutical composition comprising an effective amount of AMP 579 and aldose reductase inhibitor. In this aspect and all other aspects described herein, an aldose reductase inhibitor is selected from the group consisting of: epalrestat; 3,4-dihydro-2,8-diisopropyl-3-thioxo-2H-1,4-benzoxazine-4-acetic acid; 2,7-difluoro-spiro(9H-fluorene-9,4′-imidazolidine)-2′,5′-dione; 3-[(4-bromo-2-fluorophenyl)methyl]-7-chloro-3,4-dihydro-2,4-dioxo-1(2H)-q-uinazoline acetic acid; 6-fluoro-2,3-dihydro-2′,5′-dioxo-spiro[4H-1-benzopyran-4,4′-imidazolidine]-2-carboxamide; zopolrestat; sorbini; and 1-[(3-bromo-2-benzofuranyl)sulfonyl]-2,4-imidazolidinedione.

Another aspect of the present invention relates to methods for treating or preventing a subject with or at risk of having cardiac dysfunction comprising administering to the subject a pharmaceutical composition comprising an effective amount of an effective amount of AMP 579 and aldose reductase inhibitor.

Another aspect of the present invention relates to methods for treating or preventing a subject with or at risk of having cardiac dysfunction comprising administering to the subject a pharmaceutical composition comprising an effective amount of an effective amount of AMP 579 and β-blocker.

In all aspect as described herein, the pharmaceutical composition described herein can comprise a β-blocker and/or an aldose reductase inhibitor.

### BRIEF DESCRIPTION OF FIGURES

FIGS. 1A-C shows generation of the transgenic system to induce cardiac A₁-AR expression. FIG. 1A is a schematic depiction of the two component transgenic system to induce cardiac A₁-AR expression. FIG. 1B shows A₁-AR transgene expression in five founder lines. Total ventricular protein extracts from six-week-old male mice were immunoblotted with anti-A₁-AR antibody. FIG. 1C shows myocardial expression of A₁-AR by radioligand binding. Myocardium membranes from 6 week-old mice were incubated with A₁-AR radioligand, [³H]DPCPX. Nonspecific binding was measured in the presence of 100 uM R-PIA. Figures are representative of at least three independent experiments.

FIGS. 2A-2D show expression of A₁-AR expression in transgenic mice. FIG. 2A shows constitutive induction (Con) and doxycyline (DOX) inhibition of A₁-AR expression in six-week-old male transgenic mice. FIG. 2B shows adult A₁-AR induction. DOX was removed from transgenic mice at 3 weeks of age to induce A₁-AR expression (A₁-TG_{Ind}). Cardiac A₁-AR expression was determined when mice reached 4 weeks and 6 weeks of age. FIG. 2C shows similar myocardium A₁-AR expression in 6 week-old A₁-TG_{Con} and A₁-TG_{Ind} male mice. FIG. 2D shows A₁-AR radioligand binding. Binding assay was performed in triplicate and nonspecific binding was measured in the presence of 100 μM R-PIA. Data were presented as fmol of bound ³H DPCPX ligand per mg of membrane protein. Figures are representative of at least three independent experiments.

FIGS. 3A-3B show survival of A₁-AR transgenic mice. FIG. 3A show A₁-AR induction of scheme. FIG. 3B shows a Kaplan-Meier survival curve for transgenic line B and line C constitutively expressing A₁-AR (A₁-TG_{Con}) or with A₁-AR induced at three weeks of age by removing DOX (A₁-TG_{Ind}). For both line B and line C, P<0.01 for A₁-TG_{Ind} vs. A₁-TG_{Con}. Also, P<0.01 for line B A₁-TG_{Con} vs. line C A₁-TG_{Con}.

FIGS. 4A-4C show mouse myocardium of A₁-TG_{Con} and A₁-TG_{Ind} transgenic mice. FIG. 4A shows horizontal slices (upper), Picro-sirius Red staining (middle) and collagen gene expression (lower table) of 6 week-old mouse myocardium. FIG. 4B shows horizontal slice, Picro-sirius Red staining and collagen gene expression of 20 week-old mouse myocardium. A₁-TG_{Con} mice did not survive at 20 weeks. Minimum two animals of each genotype and age and five independent high-power fields of stained images were analyzed using Image-Pro Plus Software. Representative scaled photographic images are shown. FIG. 4C shows inhibition of Akt phosphorylation in A₁-AR expressing myocardium. Ventricular extracts from 6 week-old male mouse were probed with antibodies against phospho-Thr308 Akt, total Akt, actin and A₁-AR. Akt phosphorylation is normalized to actin (WT, n=7; A₁-TG_{Con}, n=7; A₁-TG_{Ind}, n=4). Data shown are mean±SEM. *P<0.05 vs. age-matched WT.

FIGS. 5A-5G show results of aortic banding in six week-old A₁-TG_{Ind} mice. Echocardiography, harvesting and biochemical characterizations were performed four weeks after surgery. FIG. 5A shows VW/BW ratio. FIG. 5B shows lung/BW ratio. FIG. 5C shows fractional shortening percentage. n-7-10 mice for each group *P<0.01 sham vs. banding, †P<0.01 WT-banding vs. A₁-TG banding. FIG. 5D shows relative expression (normalized to sham mice) of SERCA, PLB and collagen subtypes. *P<0.01 WT vs. A₁-TG. FIG. 5E shows picro-sirius Red staining of wild-type and A₁-TG_{Ind} mouse myocardium after banding. Five hearts from each group were stained and five independent 100× magnified fields were analyzed. Both wild-type and A₁-TG_{Ind} mouse myocardium showed enhanced staining after banding. Representative scaled photographic images are shown. FIG. 5F shows cFos and FIG. 5G shows EGR-1 gene expression in A₁-TG_{Ind} and wild-type mouse hearts at steady-state or after Langendorff perfusion (6 weeks-old male mice). Relative expression normalized to non-perfused WT mice is shown. *Steady-State (N=6) vs. Perfusion (N=3), P<0.01; †Perfusion WT vs. Perfusion A₁-TG_{Ind} (N=3,), P<0.01. The experiment was repeated twice.

FIGS. 6A-6F show that DOX treatment reversed cardiomyopathy in A₁-TG_{Con} mice. FIG. 6A shows horizontal slices of myocardium and Picro-sirius Red staining showed enlargement of the left ventricular cavity and fibrosis in 3 week-old A₁-TG_{Con} mice. FIG. 6B is a schematic diagram showing that at 3 weeks of age, A₁-TG_{Con} mice were fed with DOX diets. FIGS. 6C and 6D show the results of an assessment of cardiac function at 12 weeks of age (WT, n=11; A₁-TG_{Con}, n=4; A₁-TG_{Con}+DOX, n=8). FIG. 6C shows VW/BW ratio (mg/g) and Lung/BW ratio (mg/g) and FIG. 6D shows echocardiographic parameters (EDD, ESD and Fractional Shortening), *P<0.01 vs. WT, †P<0.01 vs. A₁-TG_{Con}. FIG. 6E shows the relative expression (normalized to WT mice) of SERCA, PLB, ANP and collagen subtypes. *P<0.01 vs. WT, †P<0.01 vs. A₁-TG_{Con}. FIG. 6F shows DOX treatment enhanced the survival of A₁-TG_{Con} mice. Kaplan-Meier survival curve for A₁-TG_{Con} mice treated with or without DOX at 3 weeks of age. P<0.01 for A₁-TG_{Con} vs. A₁-TG_{Con}+DOX.

FIG. 7 shows quantitative PCR analysis of genomic copies of inserted transgene. To quantify the number of transgenes inserted into the genome, 40 ng of genomic DNA from wild-type, A₁-TG line B and A₁-TG line C mice were used in real-time PCR reaction with a primer set that is specific for both human and mouse A₁-AR. Each experimental group was performed in triplicate and repeated three times. Data are presented as relative fold changes to the endogenous mouse A₁-AR gene.

FIG. 8 shows quantitative PCR analysis of A₁-AR transgene expression. Total RNA was extracted from the bi-ventricular tissues of wild-type, A₁-TG line B and A₁-TG line C mice. 10 μg total RNA was used to synthesize double-stranded cDNA and were then used in real-time PCR reaction with primers specific for A₁-AR and for actin. Each experimental group was performed in triplicate and repeated three times. Each experimental group was performed in triplicate. The ΔCT method was used to quantify the results, which are presented as relative fold changes to the actin gene.

FIG. 9 shows ventricular weight/body weight ratio (VW/BW) of wild type mice (WT), mice constitutively expressing tTA transactivating factor (tTA) and wild type mice on 300 mg/kg doxycyline diets (DOX). 7-10 12-weeks old mice from each group were measured. No significance was detected at p-value setting of P<0.05.

FIG. 10 shows fractional shortening (FS) in wild type mice (WT), mice constitutively expressing tTA transactivating factor (tTA) and wild type mice on 300 mg/kg doxycyline diets (DOX) inhibition of A₁-AR expression in. Echocardiography were performed on 7-10 12-weeks-old male mice. No significance was detected at p-value setting of P<0.05.

FIG. 11 shows kinase phosphorylation in A₁-AR expressing myocardium. Ventricular extracts from 6 weeks-old male mice with indicated transgenes were probed with antibodies against phospho-Ser473 Akt, phospho-JNK, phospho-P38, phospho-ERK and actin. Data represent one of three independent experiments.

FIG. 12 shows the amino acid conservation between human A₁-AR protein sequence (SEQ ID NO: 23) and mouse A₁-AR protein sequence (SEQ ID NO: 24).

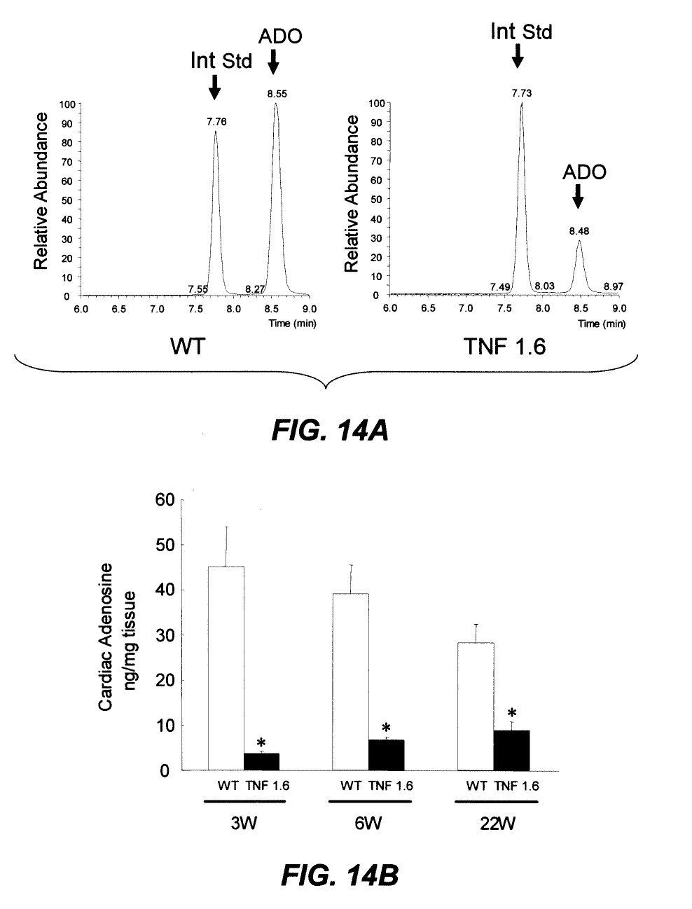

FIG. 13 shows DOX treatment reversed cardiac phenotype in A₁-TG_{Con} mice. Horizontal slice of myocardium and Picro-sirius Red staining from 12-weeks old mice are shown.

FIGS. 14A-14B show adenosine level for TNF 1.6 transgenic mice and age-matched WT mice. FIG. 14A shows HPLC/mass spectrometry analysis for adenosine level in the ventricles of TNF 1.6 and age-matched WT mice. FIG. 14B shows the average adenosine levels in 3 weeks-old, 6 weeks-old and 22 weeks-old wild type and TNF 1.6 mice. Values were mean±SEM (n=3-6) and analyzed with non-parametric method. *P<0.05 vs. WT.

FIG. 15A-15D shows A₁ and A_{2A} receptor expression in wild type and TNF 1.6 transgenic mice. FIG. 15A shows immunoblotting for A₁ and A_{2A} receptors in 6 week old wild type and TNF 1.6 mice. Values were mean±SEM (n=4) and analyzed with non-parametric method. *P<0.05 vs. age-matched WT. FIG. 15B shows the detection of myocardial expression of A₁ receptor by radio-ligand binding. Left graph: Titration curve of A₁ binding in myocardium. Right graph: Myocardium membranes were prepared from 6 week old wild type or TNF 1.6 male mice. A₁ receptor binding was determined. Values are mean±SEM (n=5) and analyzed with non-parametric method. *P<0.05 vs. age-matched WT. FIG. 15C shows immunoblotting for A₁ receptors in wild type, TNF 1.6 and TNF 1.6 mice with TNFα receptor 1 ablation (TNFR1KO). FIG. 15D shows the localization of A₁ receptor in wild type and TNF 1.6 myocardium by immunohistochemistry. Specificity is confirmed by competition with antigenic peptide (1 ug/1 ul antibody).

FIGS. 16A-16B show the cardiac response of wild type (WT) and TNF 1.6 transgenic mice to an adenosine analogue or AR agonists. Under anesthetization, a 1.4 F micromanometer catheter (Millar Instruments) was inserted into the left ventricle through the right carotid artery. Chronotropic and functional responsiveness to adenosine analogue or AR agonists were recorded. FIG. 16A shows chronotropic responses which were recorded at baseline and 10 minutes after injection of the Adenosine analogue, 2-chloroadenosine (CADO), the A₁ receptor selective agonist, 2-chloro-N⁶-cyclopentanyladenosine (CPA) and the A_{2A} receptor selective agonist, 2-p-(2-carboxyethyl)phenethylamino-5′-N-ethylcarboxamino adenosine hydrochloride (CGS21680). FIG. 16B shows the arterial pressure and cardiac contractility of the mice after CPA injection. Values are mean±SEM (n=5). *P<0.05 vs. age-matched WT. The graph measured the slope (in vivo chronotropic responsiveness) from baseline to 10 minutes after drug administration in wild-type and TNF 1.6 mice. Values were mean±SEM (n=5) and compared using ANOVA General Linear Model with repeated measures. *P<0.05 vs. age-matched WT in slope change. The graph in FIG. 16B measured the slope (arterial pressure or cardiac contractility) from baseline to 10 minutes after drug administration in wild-type and TNF1.6 mice. Values were mean±SEM (n=5) and compared using ANOVA General Linear Model with repeated measures. *P<0.05 vs. age-matched WT in slope change.

FIGS. 17A-17C shows production of adenosine (ADO) and fractional shortening (FS) in TNF 1.6 mice as compared with WT. FIG. 17A shows the proportional changes of myocardial production of adenosine (ADO) and fractional shortening (FS) in TNF 1.6 mice as compared with WT. Values were mean±SEM (n=6) and analyzed with non-parametric method. *P<0.01 vs. TNF 1.6. †P<0.01 vs. WT. FIG. 17B shows the correlation between ADO and FS. A strong positive correlation was observed among them. Correlation value was obtained using Linear Regression. FIG. 17C shows an Elisa assay for TNFα. Values were mean±SEM (n=3-6) and analyzed with non-parametric method. ‡P<0.05 vs. TNF 1.6.

FIGS. 18A-18B show relative expression levels of products produced from Myocardial tissue from TNF 1.6 and WT mice. FIG. 18A shows a summary of pathways of adenosine. 5′-NUC: 5′-nucleotidase, ADA: adenosine deaminase, ADP: Adenosine 5′-diphosphate, AK: adenosine kinase, AMP: Adenosine 5′-monophosphate, AMP-DA: AMP deaminase, ATP: Adenosine 5′-triphosphate, IMP: Adenosine 5′-monophosphate, PNP: purine nucleoside phosphorylase, SAM: S-adenosylmethionine, SAH: S-adenosylhomocysteine, XDH/XO: xanthine dehydrogenase/xanthine oxidase. FIG. 18B shows the relative expression level of myocardial productions of products that are correlated with adenosine pathways. Each measurement was normalized to WT (WT=1) and plotted on the same graph. Values are mean±SEM (n=3-6) and analyzed with non-parametric method. *P<0.01 vs. age-matched WT.

FIGS. 19A-19D shows the relative expression levels of different cardiac related gene products in TNF 1.6 and WT mice. FIG. 19A shows the relative expression levels of Atp5j and Atp5o genes in wild type and TNF 1.6 mice. Atp5j and Atp5o expression were normalized to WT (WT=1) and plotted on the same graph. Values are mean±SEM (n=3-6) and analyzed with non-parametric method. *P<0.01 vs. age-matched WT. FIG. 19B shows the real time PCR analysis of relative gene expression of ectonucleotide pyrophosphatase/phosphodiesterase 2 (Enpp2) in wild type and TNF 1.6 mice. Values were mean±SEM (n=3-6) and analyzed with non-parametric method. *P<0.01 vs. age-matched WT. FIG. 19C shows the relative expression of purine nucleoside phosphorylase (PNP) or xanthine oxidase (XO) genes in wild type and TNF 1.6 mice. Real-time PCR values were mean±SEM (n=3-6) and analyzed with non-parametric method. *P<0.01 vs. age-matched WT. (19D) Immunoblotting for XO and ENPP2 in mouse heart lysates of 6 week old wild type and TNF 1.6 mice. Values were mean±SEM (n=5) and analyzed with non-parametric method. *P<0.05 vs. age-matched WT.

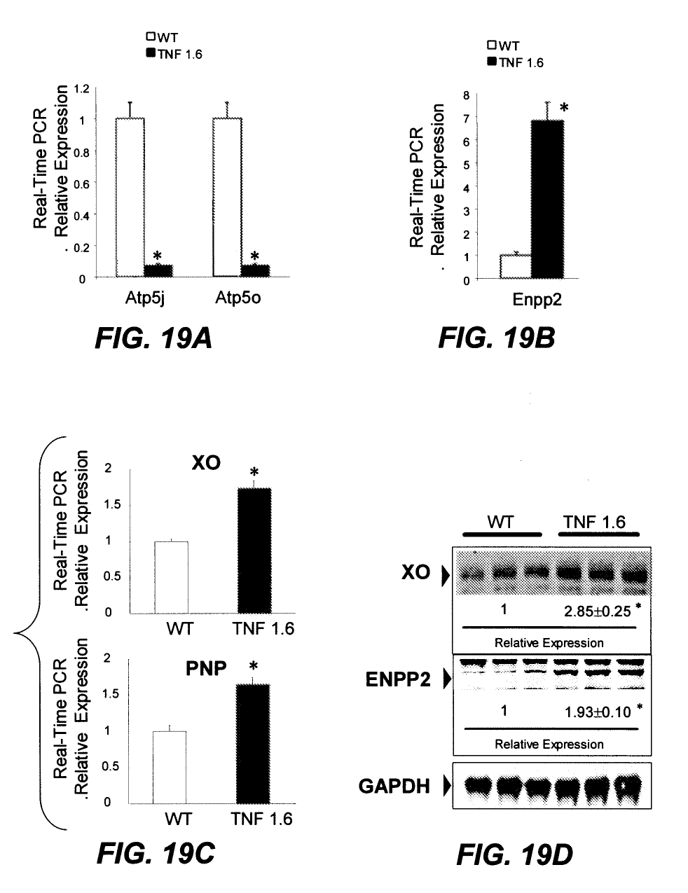

FIGS. 20A-20C show the creation and characteristics of transgenic mice expressing cardiac-specific A_{2A}-R. FIG. 20A shows A_{2A}-R transgenic founder lines express low and high levels of A_{2A}-R. Total ventricular protein extracts from six-week-old male mice were immunoblotted with anti-A_{2A}-R antibody. FIG. 20B shows A_{2A}-R transgenic founder lines express low and high levels of A_{2A}-R mRNA. DNAse-treated ventricular total RNA were used in real-time PCR. FIG. 20C shows the morphology of isolated myocytes and their length and width measurements. 20× images are shown. Values are means+/−SE. n=38-56 myocytes pooled from four to five mice.

FIGS. 21A-21B show the creation and characteristics of double transgenic mice overexpressing both A₁-R and A_{2A}-R. FIG. 21A shows A₁-R and A_{2A}-R expression in WT, A₁-TG, A_{2A}-TG and A₁/A_{2A}-TG mice. Ventricular extracts from 8 week old male mice were probed with indicated antibodies. FIG. 21B shows horizontal sections of mouse hearts stained with Haematoxylin-Eosin. Minimum two animals (8 week old male) of each genotype were stained and representative scaled 1× photographic images are shown.

FIGS. 22A-22C show A_{2A}-AR expression improves cardiac function and hemodynamics without affecting heart rate in A₁-TG mice. FIG. 22A shows the percent fractional shortening of indicated mouse groups (WT, A_{2A}Tg, A₁-Tg and A₁/A_{2A}-Tg mice); FIG. 22B shows the heart rate of indicated mouse groups; FIG. 22C shows the (+) DP/DT; FIG. 22D shows the (−)DP/DT. *P<0.001 vs. WT, †P<0.001 vs. A₁TG. (n=15-21, SE, male 8-12 week old mice).

FIGS. 23A-23D show animal survival, calcium handling and gene expression in A₁-TG and A_{2A}-TG mice. FIG. 23A shows Kaplan-Meier survival curve for A1-R transgenic lines co-expressing high levels of A_{2A}-R (A_{2A}-TGHi) A₁ vs. A₁/A_{2A} p<0.001. FIG. 23B shows Kaplan-Meier survival curve for A₁-R transgenic lines co-expressing low levels of A_{2A}-R (A_{2A}-TGlo) A₁ vs. A₁/A_{2A} NS. FIG. 23C shows the representative tracings of myocyte Ca2+ transients and contractions. Detailed calculations are shown in Table 6. FIG. 23D shows ventricular extracts from 8 week-old male mice were probed with indicated antibodies. SERCA2 and Gαi2 signals were normalized to average intensity in WT hearts (n=4-7) and were analyzed by one-way analysis of variance followed by Dunnett's test. Analyzed values were mean+/−SEM, *P<0.05 vs. WT.

### DETAILED DESCRIPTION OF INVENTION

The present invention is based on the discovery that the selective activation of A₁ adenosine receptor (A₁-AR) or selective activation of A_{2A} adenosine receptor (A_{2A}-AR) compromised cardiac function, and that this compromised cardiac function caused by single overexpression of A₁-AR or single overexpression of A_{2A}-AR was due to altered calcium homeostasis.

Activation of the A₁-AR in pregnant mice has been shown to inhibit cardiac cell proliferation and leads to cardiac hypoplasia³¹. The inventors assessed if changes in the cardiac phenotype resulting from moderate A₁-AR overexpression might be due, at least in part, to activation of the A₁-AR transgene in the early heart tube.³² The inventors tested this by creating mice with inducible overexpression of the A₁-AR using a tetracycline (Tet)-based system in which expression could be regulated throughout cardiac development.³³ Surprisingly, both constitutive and controlled overexpression of the A₁-AR resulted in the development of a reversible dilated cardiomyopathy.

The inventors discovered long-term and/or chronic overexpression of A₁-AR resulted in a decrease in calcium uptake by cardiomyocytes, leading to intolerance of the stress associated with pressure overload (such as that detected in a model of pressure stress known in the art as aortic banding) and rapid onset of cardiac failure and death. Cardiac dysfunction and cardiac enlargement was detected in a mouse genetically altered to constitutively overexpress or induced-overexpression A₁-AR expression and resulted in diminished left ventricular function with increased age of the mice. In contrast, the inventors also discovered long-term and/or chronic overexpression of A_{2A}-AR resulted in an increase in calcium uptake by cardiomyocytes, which resulted in short-term increase in muscle contractibility, but long-term leads to death of cardiomyocytes and compromised cardiac function such as congestive heart failure and decreased heart rate.

The inventors further discovered they could prevent and rescue the A₁-AR activation or overexpression mediated compromised cardiac function by also simultaneously overexpressing the A_{A2}-AR. Thus, one embodiment the present invention relates to the discovery that simultaneous activation of both the A₁- and A_{2A}-AR is a useful and safe cardioprotective strategy as compared to activation of only the A₁-AR alone or the A_{2A}-AR alone.

In another aspect of the invention, the inventors have discovered that adenosine and A₁ adenosine receptors contribute to pathobiology of heart muscle dysfunction in murine models of heart failure and chronic heart failure that are known to be applicable to the similar conditions in humans. The present invention therefore relates to adenosine therapeutics, such as adenosine and adenosine receptor agonists and antagonists, in particular agonists and/or antagonists targeting A₁ and A_{2A}-adenosine receptors.

One aspect of the present invention relates to the use of an agent which simultaneously activates both the A₁ and A_{2A} adenosine receptors to prevent the comprised cardiac function which occurs with the use of a selective A₁ adenosine receptor agonist alone. Typically, use of an A₁-AR agonist alone is commonly used as cardiac-protective treatments to prevent myocardial ischemia, where agents which activate A₁-AR are used to mimic the effects of preconditioning and increasing myocardial resistance to ischemia.

Another aspect of the present invention relates to methods to treat a subject, such as a human subject, with compromised cardiac function with a pharmaceutical composition which targets multiple adenosine receptors simultaneously in a stoichiometric relationship (i.e. each AR receptor is targeted to about the same extent). In particular, in some embodiments of this aspect of the present invention relates to a pharmaceutical composition comprising at least one agent which activates both the A₁-AR and the A_{2A}-AR in a stoichiometric relationship, or a pharmaceutical composition comprising at least one agent which activates A₁-AR and at least one agent which activates A_{2A}-AR in a stoichiometric relationship. Stated another way, in some embodiments a pharmaceutical composition as disclosed herein comprises an agent or agents which can activate the A₁-AR and also activate the A_{2A}-AR, where the level of biological activation of A₁-AR is about the same as the level of biological activation of A_{2A}-AR.

In some embodiments where a pharmaceutical composition comprises at least one agent which activates the A₁-AR and at least one agent which activates the A_{2A}-AR, the amount of an agent which activates A_{2A}-AR is an amount that counteracts or normalizes the cardiac dysfunction caused by the agent that activates A₁-AR.

Another aspect of the present invention as discussed herein relates to the administration to a subject a pharmaceutical composition comprising at least one agent which activates the A₁-AR and also activates the A_{2A}-AR in a stoichiometric relationship for the treatment for cardiac dysfunction, for example, but not limited to, for the treatment of a subject with myocardial infarction, such as acute myocardial infarction, coronary ischemia, or congestive heart failure and for the treatment of subjects undergoing percutaneous coronary intervention.

Another aspect of the present invention provides methods to screen for agents which are co-agonists or co-antagonists for the A₁- and A_{2A}-adenosine receptors, and in particular agonists which are stoichiometrically balanced agonists of A₁-AR and/or A_{2A}-AR, such as for example agents which activate the A₁-AR to the same amount which activates A_{2A}-AR. Another aspect of the present invention relates to methods for identifying agents that target A₁ and A₂ adenosine receptors simultaneously.

### DEFINITIONS

For convenience, certain terms employed in the entire application (including the specification, examples, and appended claims) are collected here. Unless defined otherwise, all technical and scientific terms used herein have the same meaning as commonly understood by one of ordinary skill in the art to which this invention belongs.

The term “agent” refers to any entity which is normally absent or not present at the levels being administered, in the cell. Agent may be selected from a group comprising; chemicals; small molecules; nucleic acid sequences; nucleic acid analogues; proteins; peptides; aptamers; antibodies; or fragments thereof. A nucleic acid sequence may be RNA or DNA, and may be single or double stranded, and can be selected from a group comprising; nucleic acid encoding a protein of interest, oligonucleotides, nucleic acid analogues, for example peptide-nucleic acid (PNA), pseudo-complementary PNA (pc-PNA), locked nucleic acid (LNA), etc. Such nucleic acid sequences include, for example, but not limited to, nucleic acid sequence encoding proteins, for example that act as transcriptional repressors, antisense molecules, ribozymes, small inhibitory nucleic acid sequences, for example but not limited to RNAi, shRNAi, siRNA, micro RNAi (mRNAi), antisense oligonucleotides etc. A protein and/or peptide or fragment thereof can be any protein of interest, for example, but not limited to; mutated proteins; therapeutic proteins; truncated proteins, wherein the protein is normally absent or expressed at lower levels in the cell. Proteins can also be selected from a group comprising; mutated proteins, genetically engineered proteins, peptides, synthetic peptides, recombinant proteins, chimeric proteins, antibodies, midibodies, tribodies, humanized proteins, humanized antibodies, chimeric antibodies, modified proteins and fragments thereof. The agent may be applied to the media, where it contacts the cell and induces its effects. Alternatively, the agent may be intracellular within the cell as a result of introduction of the nucleic acid sequence into the cell and its transcription resulting in the production of the nucleic acid and/or protein environmental stimuli within the cell. In some embodiments, the agent is any chemical, entity or moiety, including without limitation synthetic and naturally-occurring non-proteinaceous entities. In certain embodiments the agent is a small molecule having a chemical moiety. For example, chemical moieties included unsubstituted or substituted alkyl, aromatic, or heterocyclyl moieties including macrolides, leptomycins and related natural products or analogues thereof. Agents can be known to have a desired activity and/or property, or can be selected from a library of diverse compounds.

The term “A₁-AR” and “A₁-A receptor” and “A₁ adenosine receptor” are used interchangeably herein with A₁-AR” and “A₁-A receptor” and “A₁ adenosine receptor”, and refer to the A₁ adenosine receptor, also commonly known as alias ADORA₁ or RDC7 by persons of ordinary skill in the art. By way of reference example only, the human gene for A₁-AR is GenBank number NM_{—}000674 (SEQ ID NO: 25), which encodes the human protein (amino acid) sequence for A₁-AR which is NP_{—}000665 (SEQ ID NO: 26).

The term “A_{2A}-AR” and “A_{2A}-A receptor” and “A_{2A} adenosine receptor” are used interchangeably herein with A_{2A}-AR” and “A2-A receptor” and “A_{2A} adenosine receptor”, and refer to the A_{2A} adenosine receptor, also commonly known as alias ADORA_{2A}, ADORA₂, and RDC8 by persons of ordinary skill in the art. By way of reference example only, the human gene for A_{2A}-AR is GenBank number NM_{—}000675 (SEQ ID NO: 27), which encodes the human protein (amino acid) sequence for A_{2A}-AR which is (SEQ ID NO: 28).

The term “agonist” as used herein refers to any agent or entity capable of activating the expression or biological activity of a protein, polypeptide portion thereof, or polynucleotide. Thus, an agonist can operate to increase the transcription, translation, post-transcriptional or post-translational processing or otherwise activate the activity of the protein, polypeptide or polynucleotide in any way, such as functioning as a ligand to activate a receptor or via other forms of direct or indirect action. By way of example only, an agonist which activates the A₁-AR can be any entity or agent which functions as a ligand for A₁-AR, such as a ligand which binds to the active site of the A₁-AR, or alternatively any agent which interacts with the A₁-AR (at the active site or at a non-active site) to initiate downstream signalling of the A₁-AR. Similarly, and by way of example only, an agonist which activates the A_{2A}-AR can be any entity or agent which functions as a ligand for A_{2A}-AR, such as a ligand which binds to the active site of the A_{2A}-AR, or alternatively any agent which interacts with the A_{2A}-AR (at the active site or at a non-active site) to initiate downstream signalling of the A_{2A}-AR. An agonist can be, for example a nucleic acid, peptide, or any other suitable chemical compound or molecule or any combination of these. Additionally, it will be understood that in indirectly activating the activity of a protein, polypeptide of polynucleotide, an agonist may affect the activity of the cellular molecules which may in turn act as regulators or the protein, polypeptide or polynucleotide itself. Similarly, an agonist may affect the activity of molecules which are themselves subject to the regulation or modulation by the protein, polypeptide of polynucleotide. An agonist is also referred to herein as an “activating agent”.

The term “antagonist” as used herein refers to any agent or entity capable of inhibiting the expression or biological activity of a protein, polypeptide portion thereof, or polynucleotide. Thus, the antagonist may operate to prevent transcription, translation, post-transcriptional or post-translational processing or otherwise inhibit the activity of the protein, polypeptide or polynucleotide in any way, such as functioning as a ligand to activate a receptor or via other forms of direct or indirect action. By way of example only, an antagonist which inhibits the A₁-AR can be any entity or agent which functions as a to competitively block the active site for A₁-AR, or alternatively any agent which is a non-competitive inhibitor of A₁-AR which interacts at a region of A₁-AR which is not the active site) to inhibit or reduce downstream signalling of the A₁-AR. Similarly, and by way of example only, an antagonist which inhibits the A_{2A}-AR can be any entity or agent which functions as a to competitively block the active site for A_{2A}-AR, or alternatively any agent which is a non-competitive inhibitor of A_{2A}-AR which interacts at a region of A_{2A}-AR which is not the active site) to inhibit or reduce downstream signalling of the A_{2A}-AR. In some embodiments, an antagonist can indirectly inhibit the A₁-AR and/or A_{2A}-AR by inhibiting an activator the A₁-AR and/or the A_{2A}-AR respectively, or inhibit the upstream signaling pathways of A₁-AR and/or the A_{2A}-AR. An antagonist may for example, be any agent, such as but not limited to a nucleic acid, peptide, or any other suitable chemical compound or molecule or any combination of these. Additionally, it will be understood that in indirectly impairing the activity of a protein, polypeptide of polynucleotide, the antagonist may affect the activity of the cellular molecules which may in turn act as regulators or the protein, polypeptide or polynucleotide itself. Similarly, the antagonist may affect the activity of molecules which are themselves subject to the regulation or modulation by the protein, polypeptide of polynucleotide.

The terms “activate” or “increased” or “increase” as used in the context of biological activity of a protein (i.e. of A₁-AR or A_{2A}-AR) herein generally means an increase in the biological function of the protein (i.e. A₁-AR or A_{2A}-AR) by a statically significant amount relative to in the absence of an agonist or activator agent. For the avoidance of doubt, an “increase” of activity, or “activation” of a protein means a statistically significant increase of at least about 10% as compared to the absence of an agonist or activator agent, including an increase of at least about 20%, at least about 30%, at least about 40%, at least about 50%, at least about 60%, at least about 70%, at least about 80%, at least about 90%, at least about 100% or more, including, for example at least 2-fold, at least 3-fold, at least 4-fold, at least 5-fold, at least 10-fold increase or greater as compared to in the absence of an agonist or activating agent, as that term is defined herein.

The term “inhibit” or “reduced” or “reduce” or “decrease” as used herein generally means to inhibit or decrease the biological function of the protein (i.e. A₁-AR or A_{2A}-AR) by a statistically significant amount relative to in the absence of an inhibitor or antagonist. However, for avoidance of doubt, “inhibit” means statistically significant decrease in activity of the biological function of a protein by at least about 10% as compared to in the absence of an inhibitor or antagonist, for example a decrease by at least about 20%, at least about 30%, at least about 40%, at least about 50%, or least about 60%, or least about 70%, or least about 80%, at least about 90% or more, up to and including a 100% inhibition (i.e. complete absence of protein biological function as compared to the biological function of the protein (i.e. of A₁-AR or A_{2A}-AR) in the absence of an inhibitor or antagonist), or any decrease in biological activity of the protein (i.e. of A₁-AR or A_{2A}-AR) between 10-100% as compared to a in the absence of an inhibitor or antagonist, as that term is defined herein.

A “ligand” as used herein refers to an entity or molecule that binds to another, and typically refers to a soluble molecule or molecule in a cytoplasm which binds to a receptor and activates the receptor to trigger downstream signaling events. For example the endogenous ligand which binds to A₁-AR and A_{2A}-AR is adenosine.

The term “adenosine A₁/A_{2A} agonist” or “compound having adenosine A₁/A_{2A} agonistic activity” or a “A₁/A_{2A} co-agonist” as used herein refers to an agent which functions as an agonist for both the A₁-AR and A_{2A}-AR subtypes of adenosine receptors, for example an agent which activate the A₁-AR and the A_{2A}-AR with about the same level of activation, for example but not exclusively, activation of A₁-AR and A_{2A}-AR with a 1:1 ratio, or about a 1:1.125 ratio, or about a 1:1.25 ratio, or about a 1:1.5 ratio, or a about 1:1.75 ratio or a about 1:2 ratio, or alternatively, activates A_{2A}-AR and A₁-AR with a ratio of about a 1:1.125 ratio, or about a 1:1.25 ratio, or about a 1:1.5 ratio, or about 1:1.75 ratio or a about 1:2 ratio. As an exemplary example only, an adenosine A₁/A_{2A} agonist is, for example, but not limited to AMP 579.

The term “AMP 579” as used herein refers to the molecule [1S-[1α,2β,3β,4α(S*)]]-4-[7-[[3-chloro-2-thienyl)methyl]propyl]amino]-3H-imidazo[4,5-b]pyridin-3-yl]-N-ethyl-2,3-dihydroxycyclopentanecarboxamide, or analogues thereof or:

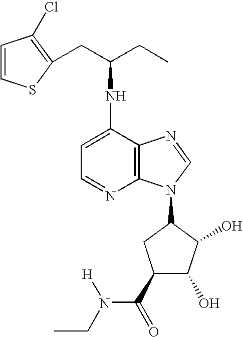

The term “selective adenosine A_{2A} receptor agonist” or “A_{2A}-AR agonist” are used interchangeably herein, refers to agonists that stimulate preferentially the adenosine A_{2A} receptor and do not stimulate substantially the adenosine A₁ receptor. Compounds can be chosen as selective A_{2A} agonists by testing for cardiovascular activity as described in Niiya, K., et al., J. Med. Chem. 35:4557-4561 (1992) and demonstrating an A₁/A₂ selectivity ratio therein defined as greater than approximately 50. As will be appreciated by one of ordinary skill in the art, other assays can be employed to screen for adenosine A_{2A} receptor agonism, or an agent which functions to activate the A_{2A}-AR.

The term “synergy” or “synergistically” are used interchangeably herein refers to the increase in the biological activity of the both the A₁-AR and the A_{2A}-AR at the same time as compared to their activation at different times.

The phrases “stoichiometric relationship” or “activation in a biological stoichiometric manner” refers to activation of two or more molecules to an equal extent. By way of example only, for every one A₁-AR protein activated, the same number of A_{2A}-AR proteins are activated. In any given population of A₁-AR and A_{2A}-AR proteins, if the ratio to A₁-AR:A_{2A}-AR is different, an agent which is capable of activating both of these receptors should have different binding affinities for A₁-AR and A_{2A}-AR such that for every one A₁-AR protein activated, the same number of A_{2A}-AR proteins are activated. Stated another way and for illustrative purposes only, if the ratio of A₁-AR:A_{2A}-AR is 2:1, a co-agonist A₁-AR and A_{2A}-AR (i.e. activates both A₁-AR and A_{2A}-AR) will have a binding affinity A₁-AR which is about half (i.e. 50%) that of the binding affinity for A_{2A}-AR, so that based on the ratio of A₁-AR:A_{2A}-AR, for every one A₁-AR protein activated, the same number of A_{2A}-AR proteins are activated.

The term “cardiovascular dysfunction” used herein refers to but is not limited to disorders and diseases of the heart and vascular system, such as congestive heart failure, myocardial infarction, ischemic diseases of the heart, all kinds of atrial and ventricular arrhythmias, hypertensive vascular diseases, peripheral vascular diseases and atherosclerosis. Heart failure and in particular Congestive Heart Failure (CHF), is one of the major causes of combined morbidity and mortality in industrialized nations. Congestive Heart Failure occurs when the heart is damaged from diseases such as e.g. high blood pressure, heart attack or arteriosclerosis and is characterized by a reduced contraction and delayed relaxation of the heart. The failing, inefficient heart eventually results in fluid retention and shortness of breath, fatigue and exercise intolerance. Diagnostic criteria for these diseases and disorders are well known and are available from The Merck Manual of Diagnosis and Therapy, 18th Edition, published by Merck Research Laboratories, 2006 (ISBN 0-911910-18-2), which is incorporated herein by reference. The current treatment of CHF is mainly directed to reduce the heart's workload by rest, by controlling sodium and water retention by means of a low sodium diet or by administering diuretics. Also the heart's activity has been tried to be influenced by administering inotropic agents, such as digoxin or vasodilators such as captopril.

The term “cardioprotection” as used herein refers to protecting against or reducing damage to the myocardium, for example prior to, during or after an ischemic attack, during reperfusion, or prior to during or after cardiac surgery. Cardioprotection methods commonly used in the art include administration of adenosine therapy, such as an A₁-AR agonist and/or A₃-AR agonist.

As used herein, the terms “treat” or “treatment” or “treating” refers to both therapeutic treatment and prophylactic or preventative measures, wherein the object is to prevent or slow the development of the disease. The term “treating” includes reducing or alleviating at least one adverse effect or symptom of a condition, disease or disorder associated with cardiac dysfunction, for example such as but not limited to cardiac dysfunction of myocardial infarction. Treatment is generally “effective” if one or more symptoms or clinical markers are reduced as that term is defined herein. Alternatively, treatment is “effective” if the progression of a disease is reduced or halted. That is, “treatment” includes not just the improvement of symptoms or markers, but also a cessation of at least slowing of progress or worsening of symptoms that would be expected in absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already diagnosed with cardiac dysfunction, as well as those likely to develop cardiac dysfunction, such as those at risk of myocardial infarction. Used in the context of cardiac dysfunction, the term treating as used herein refers to a reduction of a symptom of cardiac dysfunction of myocardial infarction and/or a reduction of at least one biochemical marker of cardiac dysfunction of myocardial infarction by at least 10%. For example but are not limited to, a reduction in a biochemical marker of cardiac dysfunction of myocardial infarction, for example a reduction in, as an illustrative example only, at least one of the following biomarkers as disclosed in U.S. Patent Application 2005/0250156, which is incorporated herein by reference, include for example, protein biomarkers in the blood such as; troponin I and T (TnI/TnT), creatine kinase-MB isoform (CKOMB), myoglobin (MYO), hsCRP, H-FABP, MPO, BNP, p-selectin, sCD40L, GPIIb/IIIa, PTF 1.2, DD, TAT, BTG, PF4, PECAM-1, TPP, IL-6, IL-18, PIGF, PaPP-A, glutathione peroxidase, plasma thioredoxin, cyctatin C, and serum deoxyribonuclease I and ATP/ADP, i.e. a reduction in the biomarkers by at least 10%. As alternative examples, a reduction in a symptom of or a reduction in the size of infarct for example for myocardial infarction by 10% or reduction in myocardial infarct, would be considered effective treatments by the methods as disclosed herein, or a reduction in a symptom of cardiac dysfunction, for example a reduction in a symptom of acute coronary symptom (ACS) or a reduction of a symptom of Congestive Heart Failure (CHF), such as for example change in symptoms include but are not limited to, a reduction in high blood pressure by at least about 10%, a reduction in chest pain by at least about 10%, an increase in heart contraction by at least about 10%, an increase in efficiency of heart pumping by about 10%, a increase in exercise tolerance by at least 10%, and decrease in shortness of breath by at least about 10% would also be considered as affective treatments by the methods as disclosed herein.

The term “effective amount” as used herein refers to the amount of therapeutic agent of pharmaceutical composition to alleviate at least one or more symptom of the disease or disorder, and relates to a sufficient amount of pharmacological composition to provide the desired effect. The phrase “therapeutically effective amount” as used herein, e.g., of any composition as disclosed herein means a sufficient amount of the composition to treat a disorder, at a reasonable benefit/risk ratio applicable to any medical treatment. The term “therapeutically effective amount” therefore refers to an amount of the composition as disclosed herein that is sufficient to effect a therapeutically or prophylatically significant reduction in a symptom or clinical marker associated with a cardiac dysfunction when administered to a typical subject who has a cardiac dysfunction, such as for example, myocardial infarction or any other disease associated with cardiac dysfunction.

With reference to the treatment of a subject with a cardiac dysfunction, the term “effective amount” as used herein refers to the amount of pharmaceutical composition comprising at least one agent that activates both the A₁-AR and A_{2A}-AR or at least one agent that activates A₁-AR and at least one agent that activates A_{2A}-AR, where the level of activation of A₁-AR and A_{2A}-AR is about the same. In the latter instance, where the pharmaceutical composition comprises at least one agent that activates A₁-AR and at least one agent that activates A_{2A}-AR, the effective amount of the agent that activates A_{2A}-AR in an amount that counteracts or normalizes the cardiac dysfunction caused by the agent that activates A₁-AR. A therapeutic “effective amount” therefore refers to an amount of a pharmaceutical composition disclosed herein that is sufficient to effect a therapeutic or prophylatically significant reduction in a symptom or clinical marker associated with cardiac dysfunction, such as myocardial infarction. As a non-limiting example, an effective amount using the methods as disclosed herein would be considered as the amount sufficient to reduce either a clinical marker or a symptom associated with cardiac dysfunction, such as myocardial infarction, for example a reduction of at least one symptom of myocardial infarction by at least 10%. An effective amount as used herein would also include an amount sufficient to prevent or delay the development of a symptom of the disease, alter the course of a symptom disease (for example but not limited to, slow the progression of a symptom of the disease), or reverse a symptom of the disease.

Thus, it is not possible to specify the exact “effective amount”. However, for any given case, an appropriate “effective amount” can be determined by one of ordinary skill in the art using only routine experimentation. The efficacy of treatment can be judged by an ordinarily skilled practitioner, for example, efficacy can be assessed in animal models of cardiac dysfunction, for example treatment of a rodent with myocardial infarction or ischemia/reperfusion injury, and any treatment or administration of the compositions or formulations that leads to a decrease of at least one symptom of the myocardial infarction, for example a prevention of a large infarct, or a reduction in the size of the infarct, or a reduction in cardiac dysfunction indicates effective treatment. In embodiments where the compositions are used for the treatment of cardiac dysfunction, the efficacy of the composition can be judged using an experimental animal model of cardiac dysfunction, e.g., mice or rats, or for example, induction of myocardial infarction in animal models, or an animal model which has been genetically modified to develop cardiac abnormalities. An effective amount can be assessed in an animal models of ischemia/reperfusion injury when administered just before reperfusion, such as disclosed in Smits et al., J Pharmacol Exp Ther 1998; 286:611-618; McVey et al., J Cardiovasc Pharmacol 1999; 33:703-710; Budde et al., Cardiovasc Res 2000; 47:294-305 and Xu et al., J Mol Cell Cardiol 2000; 32:2339-2347, which are incorporated herein in their entirety by reference.

Further, in some embodiments an experimental model could be an in vitro model, such as organ culture, cells or cell lines. When using an experimental animal model, efficacy of treatment is evidenced when a reduction in a symptom of the cardiac dysfunction, for example a reduction in the size of the infarct or prevention of such an large infarct in a treated, versus untreated animals.

A therapeutically or prophylatically significant reduction in a symptom is, e.g. at least about 10%, at least about 20%, at least about 30%, at least about 40%, at least about 50%, at least about 60%, at least about 70%, at least about 80%, at least about 90%, at least about 100%, at least about 125%, at least about 150% or more in a measured parameter as compared to a control or non-treated subject. Measured or measurable parameters include clinically detectable markers of disease, for example, elevated or depressed levels of a clinical or biological marker, as well as parameters related to a clinically accepted scale of symptoms or markers for a disease or disorder. It will be understood, however, that the total daily usage of the compositions and formulations as disclosed herein will be decided by the attending physician within the scope of sound medical judgment. The exact amount required will vary depending on factors such as the type of disease being treated.

As used herein, the terms “administering,” and “introducing” are used interchangeably and refer to the placement of the agents as disclosed herein into a subject by a method or route which results in at least partial localization of the agents at a desired site. The compounds of the present invention can be administered by any appropriate route which results in an effective treatment in the subject.

The phrases “parenteral administration” and “administered parenterally” as used herein means modes of administration other than enteral and topical administration, usually by injection, and includes, without limitation, intravenous, intramuscular, intraarterial, intrathecal, intraventricular, intracapsular, intraorbital, intracardiac, intradermal, intraperitoneal, transtracheal, subcutaneous, subcuticular, intraarticular, sub capsular, subarachnoid, intraspinal, intracerebro spinal, and intrasternal injection and infusion. The phrases “systemic administration,” “administered systemically”, “peripheral administration” and “administered peripherally” as used herein mean the administration of pharmaceutical compositions other material other than directly into the diseased tissue, such as cardiac tissue, such that it enters the subjects system and, thus, is subject to metabolism and other like processes.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. The phrase “pharmaceutically acceptable carrier” as used herein means a pharmaceutically acceptable material, composition or vehicle, such as a liquid or solid filler, diluent, excipient, solvent or encapsulating material, involved in maintaining the activity of or carrying or transporting the subject agents from one organ, or portion of the body, to another organ, or portion of the body. In addition to being “pharmaceutically acceptable” as that term is defined herein, each carrier must also be “acceptable” in the sense of being compatible with the other ingredients of the formulation. The pharmaceutical formulation contains a compound of the invention in combination with one or more pharmaceutically acceptable ingredients. The carrier can be in the form of a solid, semi-solid or liquid diluent, cream or a capsule. These pharmaceutical preparations are a further object of the invention. Usually the amount of active compounds is between 0.1-95% by weight of the preparation, preferably between 0.2-20% by weight in preparations for parenteral use and preferably between 1 and 50% by weight in preparations for oral administration. For the clinical use of the methods of the present invention, targeted delivery composition of the invention is formulated into pharmaceutical compositions or pharmaceutical formulations for parenteral administration, e.g., intravenous; mucosal, e.g., intranasal; enteral, e.g., oral; topical, e.g., transdermal; ocular, e.g., via corneal scarification or other mode of administration. The pharmaceutical composition comprises at least one agent which results in the activation of A₁-AR and A_{2A}-AR, where activation of A₁-AR and A_{2A}-AR are in a biological stoichiometric manner, and in some embodiments, in combination with one or more pharmaceutically acceptable ingredients. The carrier can be in the form of a solid, semi-solid or liquid diluent, cream or a capsule.

The phrase “pharmaceutically acceptable carrier” as used herein means a pharmaceutically acceptable material, composition or vehicle, such as a liquid or solid filler, diluent, excipient, solvent or encapsulating material, involved in carrying or transporting the subject agents from one organ, or portion of the body, to another organ, or portion of the body. Each carrier must be “acceptable” in the sense of being compatible with the other ingredients of the formulation.

The terms “patient”, “subject” and “individual” are used interchangeably herein, and refer to an animal, particularly a human, to whom treatment including prophylaxic treatment is provided. The term “subject” as used herein refers to human and non-human animals. The term “non-human animals” and “non-human mammals” are used interchangeably herein includes all vertebrates, e.g., mammals, such as non-human primates, (particularly higher primates), sheep, dog, rodent (e.g. mouse or rat), guinea pig, goat, pig, cat, rabbits, cows, and non-mammals such as chickens, amphibians, reptiles etc. In one embodiment, the subject is human. In another embodiment, the subject is an experimental animal or animal substitute as a disease model.

The term “fragment” as used herein when referred to a protein (as in “a fragment thereof”) refers to a portion of any size of that protein. The fragments may range in size from four amino acids residues to the entire amino acid sequence (that is, the “full size” sequence) minus one amino acid.

As used herein, the phrase “gene expression” is used to refer to the transcription of a gene product into mRNA and is also used to refer to the expression of the protein encoded by the gene.

As used herein, the term “overexpression” is used to refer to an increased level of the gene product and/or protein as compared to a cell or animal in the absence of overexpression.

As used herein, a “regulatory sequence”, “promoter” or “promoter region” or “promoter element” are used interchangeably herein, refers to a segment of a nucleic acid sequence, typically but not limited to DNA or RNA or analogues thereof, that controls the transcription of the nucleic acid sequence to which it is operatively linked. The promoter region includes specific sequences that are sufficient for RNA polymerase recognition, binding and transcription initiation. This portion of the promoter region is referred to as the promoter. In addition, the promoter region includes sequences which modulate this recognition, binding and transcription initiation activity of RNA polymerase. These sequences may be cis-acting or may be responsive to trans-acting factors. Promoters, depending upon the nature of the regulation may be constitutive or regulated.

The term “constitutively active promoter” refers to a promoter of a gene which is expressed at all times within a given cell. Exemplary promoters for use in mammalian cells include cytomegalovirus (CMV), and for use in prokaryotic cells include the bacteriophage T7 and T3 promoters, and the like. The term “inducible promoter” refers to a promoter of a gene which can be expressed on a given signal, for example addition or reduction of an agent. Non-limiting examples of an inducible promoter are “tet-on” and “tet-off” promoters, or promoters that are regulated in a specific tissue type.

The term “operatively linked” or “operatively associated” are used interchangeably herein, and refer to the functional relationship of the nucleic acid sequences with regulatory sequences of nucleotides, such as promoters, enhancers, transcriptional and translational stop sites, and other signal sequences. For example, operative linkage of nucleic acid sequences, typically DNA, to a regulatory sequence or promoter region refers to the physical and functional relationship between the DNA and the regulatory sequence or promoter such that the transcription of such DNA is initiated from the regulatory sequence or promoter, by an RNA polymerase that specifically recognizes, binds and transcribes the DNA. In order to optimize expression and/or in vitro transcription, it may be necessary to modify the regulatory sequence for the expression of the nucleic acid or DNA in the cell type for which it is expressed. The desirability of, or need of, such modification may be empirically determined.

The interaction of a cellular receptor (i.e. A₁-AR or A_{2A}-AR) and an agonist, such as a ligand, may be described in terms of “affinity” and “specificity”. Affinity is sometimes quantified by the equilibrium constant of complex formation. Specificity relates to the difference in affinity between the same agonist or ligand binding to different receptors (i.e. A₁-AR or A_{2A}-AR) or to different binding sites on the same cellular receptor. The terms “binding affinity” or “specifically binds” as used herein in the context of an agonist binding to a receptor (i.e. A₁-AR or A_{2A}-AR) indicates that the binding preference (e.g., affinity of the agonist for the target A₁-AR or A_{2A}-AR is at least 2 fold, more preferably at least 5 fold, and most preferably at least 10 or 20 fold over a non-specific (e.g. randomly generated molecule lacking the specifically recognized amino acid or amino acid sequence) target molecule or protein. Stated another way, the term “specifically bind” as used herein refers to the ability of an agonist to bind to a target protein with a greater affinity than non target proteins. For example, about 10%, about 20%, about 30%, about 40%, preferably about 50%, more preferably about 60%, more preferably about 70%, still more preferably about 80%, still more preferably about 90%, still more preferably about 100% or greater affinity for the target protein (i.e. A₁-AR or A_{2A}-AR) relative to non-target proteins. At a minimum, the term “specifically binds” refers to binding with a K_{d} of 10 micromolar or less, preferably 1 micromolar or less, more preferably 100 nM or less, 10 nM or less, or 1 nM or less. One example of an agonist is an A₁/A_{2A} co-agonist which specifically binds both the binds A₁-AR and A_{2A}-AR receptors.

The articles “a” and “an” are used herein to refer to one or to more than one (i.e., at least one) of the grammatical object of the article. By way of example, “an element” means one element or more than one element. Thus, in this specification and the appended claims, the singular forms “a,” “an,” and “the” include plural references unless the context clearly dictates otherwise. Thus, for example, reference to a pharmaceutical composition comprising “an agent” includes reference to two or more agents.

As used herein, the term “comprising” means that other elements can also be present in addition to the defined elements presented. The use of “comprising” indicates inclusion rather than limitation. The term “consisting of” refers to compositions, methods, and respective components thereof as described herein, which are exclusive of any element not recited in that description of the embodiment. As used herein the term “consisting essentially of” refers to those elements required for a given embodiment. The term permits the presence of elements that do not materially affect the basic and novel or functional characteristic(s) of that embodiment of the invention.

Other than in the operating examples, or where otherwise indicated, all numbers expressing quantities of ingredients or reaction conditions used herein should be understood as modified in all instances by the term “about.” The term “about” when used in connection with percentages can mean±1%.

This invention is further illustrated by the following examples which should not be construed as limiting. The contents of all references cited throughout this application, as well as the figures and tables are incorporated herein by reference.

It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims. Other features and advantages of the invention will be apparent from the following Detailed Description, the drawings, and the claims.

Agents which Activate A₁-AR and/or A_{2A}-AR

In one aspect of the present invention relates to a pharmaceutical composition, and use thereof, where the pharmaceutical composition comprises an effective amount of at least one agent which simultaneously activates both the A₁-AR and the A₂-AR, where A₁-AR and A₂-AR are activated in a biologically stoichiometric manner. In other words, in this aspect and all other aspects described herein, a pharmaceutical composition comprises an agent or agents which can activate A₁-AR and A_{2A}-AR, where the level of biological activation of A₁-AR is matched (or equal) with the level of biological activation of A_{2A}-AR.

In some embodiments in this aspect and all other aspects described herein, the pharmaceutical composition comprises an agent with dual function to activate both the A₁-AR and A_{2A}-AR simultaneously. For example, but not limited to, the AMP579 compound, which is disclosed in U.S. Patent Application No. 2004/0248928 and 2004/0122045 and are incorporated herein in their entirety by reference. AMP 579 is a co-agonist for A₁-AR and A₂-AR (herein also referred to a A₁/A_{2A} receptor agonist or A₁/A_{2A} co-agonist) and has been demonstrated to be cardioprotective when administered with a sodium-hydrogen exchanger at the time of reperfusion, as well as in animal models of ischemia/reperfusion injury when administered just before reperfusion (Smits G J, McVey M, Cox B F, Perrone M H, Clark K L: Cardioprotective effects of the novel adenosine A₁/A₂ receptor agonist AMP 579 in a porcine model of myocardial infarction. J Pharmacol Exp Ther 1998; 286:611-618; McVey M J, Smits G J, Cox B F, Kitzen J M, Clark K L, Perrone M H: Cardiovascular pharmacology of the adenosine A₁/A₂-receptor agonist AMP 579: coronary hemodynamic and cardioprotective effects in the canine myocardium. J Cardiovasc Pharmacol 1999; 33:703-710; Budde J M, Velez D A, Zhao Z-Q, Clark K L, Morris C D, Muraki S, Guyton R A, Vinten-Johansen J: Comparative study of AMP579 and adenosine in inhibition of neutrophil-mediated vascular and myocardial injury during 24 h of reperfusion. Cardiovasc Res 2000; 47:294-305 and Xu Z, Yang X-M, Cohen M V, Neumann T, Heusch G, Downey J M: Limitation of infarct size in rabbit hearts by the novel adenosine receptor agonist AMP 579 administered at reperfusion. J Mol Cell Cardiol 2000; 32:2339-2347).

In some embodiments in this aspect and all other aspects described herein, the pharmaceutical composition can comprise pharmaceutically acceptable carrier and pharmaceutically effective amounts of an agent which functions as an A₁-AR agonist and an effective amount of an agent that function as an A_{2A}-AR agonist.

In some embodiments, agents which activate A₁-AR and are useful as A₁-AR agonists in the pharmaceutical compositions and methods as disclosed herein can be selected from a group comprising adenosine agonists are described in PCT application 05003150, PCT 9850047, U.S. Patent Application 2004020248928 which are incorporated herein by reference in their entirety. Such A₁-AR agonists useful in this aspect and all other aspects described herein include, but are not limited to, A₁-AR selective agonists CCPA, CHA, ADAC, CI-IB-MECA, MRS584, MRS537, MRS1340 and DBXMA, MRS646, MRS1364 (see U.S. Pat. No. 9,850,047), AB-MECA, CPA, ADAC, GR79236, S-ENBA, IAB-MECA, R-PIA, ATL146e, CGS-21680, CV 1808, HENECA, NECA, PAPA-APEC, DITC APEC DPMA, S-PHPNECA, WRC-0470, AMP-579, IB-MECA, 2-CIADO, I-ABA, S-PIA, 2-[(2-aminoethyl aminocarbonylethyl)phenylethyl amino]-5′-N-ethyl-carboxamidoadenosine, 2-C1-IB MECA, polyadenylic acid, and any mixture or analogue or derivative thereof.

Other agents which activate A₁-AR and can be used as A₁-AR agonists in this aspect and all other aspects described herein, include for example but not limited to A₁-AR agonists selected from the group of: AB-MECA V6-4-amino benzyl-5′-N-methylcarboxamidoadenosine), CPA (N6-cyclopentyladenosine), ADAC (N6-[4-[[[4-[[[(2-aminoethyl)amino]carbonyl]methyl]-anilino]carbonyl]methyl]phenyl]adenosine), CCPA (2-chloro-N6-cyclopentyladenosine), CHA (N6-cyclohexyladenosine), GR79236 (1V6-[1S, trans,2-hydroxycyclopentyl]adenosine), S-ENBA ((2S)—N6-(2-endonorbanyl) adenosine), IAB-MECA (1V6-(4-amino-3-iodobenzyl)adenosine-5′-N-methylcarboxamidoadenosine), R-PIA (R—N6-(phenylisopropyl) adenosine), ATL146e (4-[3-[6-amino-9-(5-ethylcarbamoyl-3,4 dihydroxy [symbol] tetrahydro-furan-2-yl)-9H-purin-2-yl]-prop-2-ynyl]-cyclohexanecarboxylic acid methyl ester), CGS-21680 (APEC or 2-Lp-(2-carbonyl-ethyl)-phenyl ethyl amino]-5′-N-ethylcarboxamidoadenosine), CV 1808 (2-phenylaminoadenosine), HENECA (2-hex-1-ynyl-5′-N-ethylcarboxamido adenosine), NECA (5′-N-ethyl [symbol] carboxamido adenosine), PAPA-APEC (2-(4-[2-[(4-aminophenyl)methyl carbonyl]ethyl]phenyl)ethylamino-5′-N-ethyl carboxamidoadeno sine), DITC APEC (2-[p-(4-isothiocyanatophenyl aminothiocarbonyl-2-ethyl)-phenylethylamino]-15′-N-ethylcarboxamidoadenosine), DPMA (N6-(2(3,5-dimethoxy phenyl)-2-(2-methyl phenyl)ethyl)adenosine), S-PHPNECA ((S)-2-phenylhydroxypropynyl-5′-N ethylcarbox amidoadenosine), WRC-0470 (2 cyclohexylmethylidenehydrazinoadenosine), AMP-579 (1S-[1a,2b,3b,4a(S*)]]-4-[7 [[2-(3-chloro-2-thienyl)-1-methylpropyl]amino]-3H-imidazo[4,5-b]pyridyl-3-yl]cyclopentane carboxamide), IB-MECA (N6-(3-iodobenzyl) adenosine-5′-N methyluronamide), 2-CIADO (2-chloroadenosine), I-ABA (N6-(4-amino-3-1 iodobenzyl) adenosine), S-PIA (S—N6-(phenylisopropyl) adenosine), 2-[(2-aminoethyl aminocarbonylethyl)phenylethyl amino]-5′-N-ethyl-carboxamidoadenosine, 2-C1-IB MECA (2-chloro-Ni-(3-iodobenzyl) adenosine-5′-N-methyluronamide), polyadenylic acid, and any mixture or analogue or derivative thereof. In some embodiments, agents which are useful as agents which activate A₁-AR are, for example, 2-chloro-N6-CyClopentyladenosine (CCPA), N6-cyclohexyladenosine (CHA) and adenosine amine congener (ADAC).

In some embodiments in this aspect and all other aspects described herein, agents which activate A_{2A}-AR and are useful in the pharmaceutical compositions and methods as disclosed herein can be selected from a group for example, but not limited to, 2-(substituted amino)adenosine 5′-carboxamides, as described in U.S. Pat. No. 4,968,697; 2-(substituted amino)adenosines, described in U.S. Pat. No. 5,034,381; imidazo-[4,5-b]-pyridine derivatives, described in U.S. Pat. No. 4,977,144; and 2-(substituted alkynyl)adenosines, described in U.S. Pat. No. 5,189,027. Additional examples of selective A_{2A} receptor agonists include 2-hydrazoadenosines, described in U.S. Pat. Nos. 5,278,150 and 2-aralkoxy and 2-alkoxy adenosines, described in U.S. Pat. No. 5,140,015; 2-cyclohexylmethylenehydrazinoadenosine, 2-(3-cyclohexenyl)methylenehydrazinoadenosine, 2-isopropylmethylenehydrazinoadenosine, N-ethyl-1′-deoxy-1′-[6-amino-2-[(2-thiazolyl)ethynyl]-9H-purin-9-yl]-â-D-ribofuranuronamide, N-ethyl-1′-deoxy-1′-[6-amino-2-[hexynyl]-9H-purin-9-yl]â-D-ribofuranuronamide, 2-(1-hexyn-1-yl)adenosine-5′-N-methyluronamide, 5′-chloro-5′-deoxy-2-(1-hexyn-1-yl)adenosine, N₆-[2-(3,5-dimethoxyphenyl)-2-(2-methylphenyl)adenosine, 2-(2-phenyl)ethoxyadenosine, 2-[2-(4-methylphenyl)ethoxy]adenosine, 2-[2-(4-fluorophenyl)ethoxy]adenosine, 2-(2-(2-naphthyl)ethoxy)adenosine, 2-[p-(2-carboxyethyl)phenethylamino-5′-N-ethylcarboxamidoadenosine (CGS-21680), 2-(2-cyclohexyl)ethoxyadenosine, 2-octynyladenosine (YT-146), 2-thiazolylethynyladenosine and 2-phenethylamino-5′-N-ethylcarboxamidoadenosine (CGS-21577). The patent applications and published patent applications disclosing A_{2A}-AR agonists referred to in this paragraph are all incorporated herein in their entirety by reference.

In some embodiments in this aspect and all other aspects described herein, an agent which activates the A_{2A}-AR can be selected from the group, for example but not limited to; 2-cyclohexylmethylenehydrazinoadenosine, 2-(3-cyclohexenyl)methylenehydrazinoadenosine, 2-isopropylmethylenehydrazinoadenosine, 2-(2-phenyl)ethoxyadenosine, 2-(2-(4-methylphenyl)ethoxyadenosine, 2-(2-cyclohexyl)ethoxyadenosine, and 2-(2-(p-carboxyethyl)phenyl)ethylamino-5′-N-ethyl-carboxamidoadenosine.

In some embodiments in this aspect and all other aspects described herein, the pharmaceutical composition can comprise a pharmaceutically acceptable carrier and pharmaceutically effective amounts of an A₁-AR agonist and an A_{2A}-AR agonists, wherein the A₁ adenosine receptor agonist is conjugated with the A₂ adenosine agonist. For example, the methods for conjugation of two agents to form a binary conjugate is described in detail in PCT Patent Application No. 9966944, which is specifically incorporated by reference in its entirety.

In some embodiments in this aspect and all other aspects described herein, the pharmaceutical composition can comprise a pharmaceutically acceptable carrier and pharmaceutically effective amounts of an A₁ adenosine receptor agonist and a A₂ adenosine receptor agonists which are polypeptide and proteins. In one embodiment, the polypeptides may be an adenosine A₁-specific receptor ligand, or fragment or portion or variant thereof, and in another embodiment, the polypeptide may be an adenosine A₂-specific receptor ligand, or fragment or portion or variant thereof. In one embodiment, the polypeptides that activate A₁ and A_{2A} adenosine receptors may be conjugated, such methods of protein or polypeptide conjugation are well known in the art, and are for example, conjugation by chemical means, covalent bonds, linkers and the like. In some embodiments the conjugation may be protein fusion, the methods of which are well known in the art.

In some embodiments in this aspect and all other aspects described herein, multi-binding agents are useful in the methods and compositions as disclosed herein, for example multi-binding agents capable of activating at least two receptors for example at least two or more adenosine receptors, in particular at least two sub-types of adenosine receptors such as A₁-AR and A_{2A}-AR. Multivalent binding interactions are characterized by the concurrent interaction of multiple ligands with multiple ligand binding sites on one or more cellular receptors. Multivalent interactions differ from collections of individual monovalent interactions by imparting enhanced biological and/or therapeutic effect. Just as multivalent binding can amplify binding affinities; it can also amplify differences in binding affinities, resulting in enhanced binding specificity as well as affinity. An example of a multi-binding agent is an avimer, which relates to a peptide agent which is capable of binding to one or more sites.

In some embodiments in this aspect and all other aspects described herein, the pharmaceutical composition can comprise a pharmaceutically acceptable carrier and agent which is an avimer which activates both the A₁-AR and the A_{2A}-AR. Avimers are multi-domain proteins with multiple binding properties and are comprised typically of multiple independent binding domains linked together, such as a binding domain for A₁-AR and a binding affinity for A_{2A}-AR. As such, avimers have improved affinity and specificity for multiple receptors, such as A₁-AR and A_{2A}-AR herein as compared to conventional single epitope binding agents. In some embodiments, an agent useful in the pharmaceutical composition as disclosed herein is an avimer which is a protein or polypeptide that can bind simultaneously to A₁-AR and A₂-AR, a process known as multi-point attachment in the art. Accordingly, in some embodiments the present invention encompasses an agent which is a multi-binding agent, such as an avimer, which binds and activates the A₁-AR and also binds and activates the A_{2A}-AR. In some embodiments, such a multi-binding A₁-AR/A_{2A}-AR agent can bind A₁-AR and A_{2A}-AR with the same or different binding affinities, such that, depending on the ratio of distribution of A₁-AR:A_{2A}-AR molecules, when one A₁-AR protein is activated, one A_{2A}-AR protein is also activated.

Measurement of Biological Activity of Agents that Activate A₁-AR and A₂-AR

Agents that function to activate A_{2A}-AR (i.e. function A_{2A}-AR agonists) produce a variety of effects that depend on both the characteristics of the agent or agonist, its receptor, and the tissue bearing A_{2A} receptors. Factors relate to agonist properties are the intrinsic efficacy (E) and the equilibrium dissociation constant of the agonist-receptor complex (K_{d}).

Similarly, agents that function to activate A₁-AR (i.e. function A₁-AR agonists) produce a variety of effects that depend on both the characteristics of the agent or agonist, its receptor, and the tissue bearing A₁ receptors. Factors relate to agent properties are the intrinsic efficacy (E) and the equilibrium dissociation constant of the agent-receptor complex (K_{d}).

Similarly, an agent which functions to activate both the A₁-AR and the A_{2A}-AR simultaneously (i.e. function A₁/A_{2A}-AR co-agonists) will depend on the characteristics of the agent or agonist, its receptor, and the tissue in which the agent is present with respect to the distribution of A₁-AR and A_{2A} receptors. Factors relate to agent properties are the intrinsic efficacy (E) and the equilibrium dissociation constant of the agent-receptor complex (K_{d}).

Intrinsic efficacy (maximal efficacy) is the maximum effect that an agonist can produce if the dose is taken to its maximum. Efficacy is determined mainly by the nature of the receptor and its associated effector system. By definition, partial agonist has a lower maximal efficacy than full agonists.

The K_{d} of a drug is obtained from data generated from a saturation experiment analyzed according to a Scatchard plot (B/F versus F), which leads to a linear curve. The K_{d} is estimated as the negative reciprocal of the slope of the line of best fit, and Bₘₐₓ by the abscissa intercept of the line. The reciprocal of K_{d} measures the affinity constant (Kₐ) of the radioligand for the receptor. Thus, for a given ligand-receptor pair, the smaller the K_{d} (0.1-10 nM) the higher its affinity. Bₘₐₓ is expressed as pmol or fmol per mg tissue or protein.

When the saturation experiment is performed in the presence of a displacer (competitor), the line of best fit of the Scatchard plot can be modified in a manner that depends on the type of receptor interaction exhibited by the displacer. Two main cases exist: (1) decreased slope and unchanged Bₘₐₓ, the displacement is of the competitive type; (2) unchanged slope and unchanged displacement of the non-competitive type. Intermediate cases where both the slope and Bₘₐₓ are modified also exist.

Data generated from a displacement experiment are generally fitted by a sigmoidal curve termed the displacement or inhibition curve, that is the percentage radiolabeled ligand specifically bound versus log [displaces] in M). The abscissa of the inflexion point of the curve gives the IC₅₀ value, the concentration of displacer that displaces or inhibitor 50% of the radioactive ligand specifically bound. IC₅₀ is a measure of the inhibitor or affinity constant (Kᵢ) of the displacer for the receptor. IC₅₀ and Kᵢ are linked as follows if the displacement is of the competitive type then:

Kᵢ=IC₅₀/(1+[C*]/K_{d}*

This is the Cheng-Prusoff equation (Biochem. Pharmacol, 22:3099 (1973)). [C*] is the concentration of radioligand and K_{d}* is its dissociation constant. The duration of the biological effect of an agonist is directly related to the binding affinity of a compound. It is desirable that compounds useful in the methods as disclosed herein act as adjuncts have an effect that is long enough to produce a response without repeated administration but short enough to avoid adverse side effects.

The potency is the dose or concentration required to bring about some fraction of a compound's maximal effect (i.e., the amount of compound needed to produce a given effect). In graded dose-response measurements, the effect usually chosen is 50% of the maximum effect and the dose causing the effect is called the EC₅₀. Dose-response ratios using EC₅₀ values for an agonist for a given receptor in the absence and presence of various concentrations of an antagonist for the same receptor are determined and used to construct a Schild plot from which the K_{b} and PA₂ (−log 10K_{b}) values are determined.

The concentration of antagonist that causes 50% inhibition is known as the IC₅₀. IC₅₀ is used to determine the K_{b}, the equilibrium dissociation constant for the antagonist-receptor complex. Thus, K_{b}=[IC₅₀]/1+[A]/K_{A}

Wherein K_{A}=equilibrium dissociation constant for an agonist binding to a receptor (concentration of agonist that causes occupancy of 50% of the receptors) and [A] is the concentration of agonist.

An agent can be potent but have less intrinsic activity than another compound. Relatively potent therapeutic compounds are preferable to weak ones in that lower concentrations produce the desired effect while circumventing the effect of concentration dependent side effects.

The tissue specific factors that determine the effect of an agonist are the number of viable specific receptors in a particular tissue [RT] and the efficiency of the mechanisms that convert a stimulus (S) into an effector response. Thus, there exists for a given tissue, a complex function f(S) that determines the magnitude of the response: Response=f(S)=[f ([A]E [RT])]/([A]+K_{d})

Accordingly, a response to an agent as disclosed herein is a function of both the stimulus produced by agent interaction with the receptor and the efficiency of the transduction of that stimulus by the tissue. Stimulus is proportional to the intrinsic efficacy of the agent and the number of receptors. Consequently, variation in receptor density in different tissues can affect the stimulus for response.

In other words, the distribution or ratio of A₁-AR to A_{2A}-AR in the heart will affect how a subject will respond to a pharmaceutical composition comprising at least one agent that activates both the A₁-AR and A_{2A}-AR substantially simultaneously. For instance, a subject having a ratio of A₁-AR to A_{2A}-AR which is different from the normal distribution of A₁-AR to A_{2A}-AR will respond differently as compared to a normal distribution to a pharmaceutical composition comprising at least one agent that activates A₁-AR and A_{2A}-AR.

Furthermore, some tissues have very efficiently coupled receptors and other tissues have relatively inefficient coupled receptors. This has been termed ‘receptor reserve’ (or spare receptor) since in the first case, a maximum effect can be achieved when a relatively small fraction of the receptor is apparently occupied and, further receptor occupancy can produce no additional effect. The magnitude of the response will thus depend on the intrinsic efficacy value so that, by classical definition, full agonists (E=1) produce the maximum response for a given tissue, partial agonists produce a maximum response that is below that induced by the full agonist (0≦E≦1), and antagonists produce no visible response and block the effect of agonists (E=0). These activities can be completely dependent upon the tissue, i.e., upon the efficiency coupling. By way of an example, a low-efficacy A₁-AR agonists may be partial A₁-AR agonists in a given tissue and yet function as a full A₁-AR agonists in peripheral arteries with respect to a function such as vasodilatation.

The presence of spare receptors in a tissue increases sensitivity to an agonist. For example, if a subject has a ratio of A₁-AR to A_{2A}-AR in the heart of 1:1.5, then the heart tissue have increased sensitivity to an agent that activates A_{2A}-AR as compared to A₁-AR, provided the agents have the same binding affinity for their respective receptors (i.e. the binding affinity for the agent which activates A_{2A}-AR is the same as the binding affinity for the agent which activates A₁-AR). Thus, an important biologic consequence of spare receptors is that they allow agonists with low efficacy for receptors to produce full responses at low concentrations and therefore elicit a selective tissue response.

Thus, in one embodiment, the present intervention provides methods to administer to a subject a pharmaceutical composition comprising at least one agent which substantially simultaneously activates the A₁-AR receptor and the A_{2A}-AR, where the biological consequence of such dual activation of both the A₁-AR and A_{2A}-AR is that the activation of the A₁-AR results in a level of signalling that is within 10% of the level of signalling as a result of the activation of the A_{2A}-AR.

In some embodiments, the methods as disclosed herein allow for identifying and determining the binding affinity and agonist efficacy of an agent for A₁-AR as compared to a known full A₁-AR agonist. Then, the binding affinity of the A₁-AR activating agent can be determined. Similarly, In some embodiments, the methods as disclosed herein allow for identifying and determining the binding affinity and agonist efficacy of an agent for A_{2A}-AR as compared to a known full A_{2A}-AR agonist such as those disclosed herein, allowing the binding affinity of the A_{2A}-AR activating agent to be determined. Agents identified by this method will demonstrate partial agonist effects in the cAMP assays and a low IC as determined by affinity binding assays.

Methods to Identify and Treat Subjects Amenable to Administration of a Pharmaceutical Compositions Comprising Agents that Activate A₁-AR and A₂ₐ-AR

Another aspect of the present invention relates to methods to treat and/or prevent cardiac dysfunction in a subject, for example ischemic damage in a subject, the method comprising administering to a subject a pharmaceutical composition comprising agents which function to activate both the A₁-AR and A_{2A}-AR.

In some embodiments in this aspect and all other aspects described herein, the subject is identified to have myocardial infarction, and in some embodiments, the subject is identified to be at risk of myocardial infarction, for example the subject has cardiac dysfunction, or expresses a symptom of coronary syndrome. In some embodiments, a subject has suffered an infarction, for example the subject has ischemic damage or a myocardial infarction. In some embodiments, the subject expresses a symptom of coronary syndrome or coronary artery disease and/or has had a myocardial infarction. In another embodiment, the subject has not yet expressed a symptom of coronary syndrome or coronary artery disease, but has, for example, a family or a biological family history of the disease, or alternatively has a polymorphism which identifies them with an increased risk of developing a coronary syndrome, coronary artery disease, or an other cardiac dysfunction as that term is defined herein.

Without being bound to theory, myocardial infarction (i.e. a heart attack) can be a consequence of coronary artery disease. In some instances, coronary artery disease can occur from atherosclerosis, when arteries become narrow or hardened due to cholesterol plaque build-up, with further narrowing occurring from thrombi (blood clots) that form on the surfaces of plaques. Myocardial infarction can occurs when a coronary artery is so severely blocked that there is a significant reduction or break in the blood supply, causing damage or death to a portion of the myocardium (heart muscle). Depending on the extent of the heart muscle damage, the patient may experience significant disability or die as a result of myocardial infarction.

In alternative instances, myocardial infarction can result from a temporary contraction or spasm of a coronary artery. When this occurs, the artery narrows and the blood flow from the artery is significantly reduced or stopped. Though the cause of coronary artery spasm is still unknown, the condition can occur in both normal blood vessels and those partially blocked by plaques.

In some embodiments in this aspect and all other aspects described herein, the methods and compositions are useful for treatment of heart or cardiac dysfunctions. The terms “heart dysfunction” or “cardiac disorder” are used interchangeably herein, and refers to diseases which affect the heart. Heart dysfunctions include, but are not limited to, cardiomyopathies, cardiovascular diseases, coronary heart diseases, heart failures, hypertensive heart diseases, inflammatory heart diseases, and valvelar heart diseases.

In some embodiments in this aspect and all other aspects described herein, a heart dysfunction is a coronary heart disease. By “coronary heart disease (CHD)”, or “coronary artery disease (CAD)”, or “ischemic heart disease” or atherosclerotic heart disease” herein is meant a disease that is the end result of the accumulation of atheromatous plaques within the walls of the arteries that supply the myocardium with oxygen and nutrients. The limitation of blood flow to the heart causes ischemia of the myocardial cells. When myocardial cells die from lack of oxygen, it results in myocardial infarction (heart attack), which leads to heart muscle damage, heart muscle death and later scarring without heart muscle regrowth.

In some embodiments, the coronary heart disease is acute coronary syndrome. By “acute coronary syndrome (ACS)” herein is meant a set of signs and symptoms, usually a combination of chest pain and other features, interpreted as being the result of cardiac ischemia. The most common cause ACS is the disruption of atherosclerotic plaque in an epicardial coronary artery. The subtypes of acute coronary syndrome include unstable angina (UA, not associated with heart muscle damage), and two forms of myocardial infarction (ST segment elevation myocardial infarction (STEMI) and non-ST segment elevation myocardial infarction (NSTEMI).

In some embodiments, the heart dysfunction is heart failure. The term “heart failure”, or “congestive heart failure (CHF)”, or “congestive cardiac failure (CCF)” are used interchangeably herein and refer to a condition that results from any structural or functional cardiac disorder that impairs the ability of the heart to fill with or pump a sufficient amount of blood through the body. Heart failure can be either chronic or acute. Most heart failures are chronic and progressive illness resulting from a variety of cardiac problems, including ischemic and valvular heart disease, cardiomyopathy, hypertension, and abnormal diastolic or systolic function. However, heart failure may also develop suddenly, particularly as a complication of acute myocardial infarction or as an acute exacerbation in patients with previously compensated chronic heart failure.

In some embodiments in this aspect and all other aspects described herein, the methods and compositions are useful for a coronary protective effect and to maintain the integrity of cellular signalling.

The adenosine A₁ receptor is a member of a group of G protein-coupled receptors that hyperpolarize cells and either inhibit or promote adenylate cyclase activity and thus production of cAMP. Without being bound by theory, it is believed that adenosine A1 receptor agonists offer coronary protective effect and maintain the integrity of cellular signalling by the blockade of Ca²⁺ influx, which results in the inhibition of glutamate release and reduction of its excitatory effects at a postsynaptic level.

In some embodiments in this aspect and all other aspects described herein, subjects amenable to the administration of a pharmaceutical composition as disclosed herein is a subject identified to be at risk of myocardial infarction. Such subjects can be identified based on risk factors commonly known by persons in the art to be associated with myocardial infarction, and include for example subjects with hypertension (high blood pressure), low levels of HDL (high-density lipoproteins), or high levels of LDL (low-density lipoprotein) blood cholesterol or high levels of triglycerides, subjects with a family history of heart disease (especially with onset before age 55), aging men and women, persons with type 1 diabetes, post-menopausal women, obese subjects, subjects who smoke, and subjects with increased stress.

In some embodiments in this aspect and all other aspects described herein, the method comprises administering to a subject a pharmaceutical composition comprising at least one agent which function to activate both the A₁-AR and A_{2A}-AR for the treatment of heart dysfunction, including but is not limited to ischemic damage, such as myocardial infarction.

In some embodiments in this aspect and all other aspects described herein, a methods to treat heart dysfunctions, such as acute coronary syndromes and acute heart failures, comprises administering to a subject a pharmaceutical composition comprising agents which function to activate both the A₁-AR and A_{2A}-AR pharmaceutical composition comprising agents which function to activate both the A₁-AR and A_{2A}-AR and one or more β-blockers. By “beta blockers” or “β-blockers” or “beta adrenergic-receptor blockers” as used herein is meant a class of drugs block the action of endogenous catecholamine (such as epinephrine (adrenaline) and norepinephrine (noradrenalin)), on β-adrenergic receptors, part of the sympathetic nervous system which mediates the hyperarousal (acute stress) response. There are three known types of beta receptor, designated β₁, β₂ and β₃. β₁-Adrenergic receptors are located mainly in the heart and in the kidneys. β₂-Adrenergic receptors are located mainly in the lungs, gastrointestinal tract, liver, uterus, vascular smooth muscle, and skeletal muscle. β₃-receptors are located in fat cells. β-blockers are used for various indications, but particularly for the management of cardiac arrhythmias and cardioprotection after myocardial infarction. β-blockers that suitable for the present invention include, but is not limited to: alprenolol, carteolol, levobunolol, mepindolol, metipranolol, nanolol, oxprenolol, penbutolol, pindolol, propranolol, sotalol, timolol, acebutolol, atenolol, betaxolol, bisoprolol, esmolol, metoprolol, nebivolo, carvedilol, celiprolol, and labetaol.

In some embodiments in this aspect and all other aspects described herein, subjects amenable to the pharmaceutical compositions as disclosed herein are subjects diagnosed with myocardial infarction.

Subjects can be identified by any method to diagnose myocardial infarction (commonly referred to as a heart attack) which are commonly known by persons of ordinary skill in the art, and such subjects identified with myocardial infarction are amenable to treatment using the methods as disclosed herein, and such diagnostic methods include, for example but are not limited to; (i) blood tests to detect levels of creatine phosphokinase (CPK), aspartate aminotransferase (AST), lactate dehydrogenase (LDH) and other enzymes released during myocardial infarction; (ii) electrocardiogram (ECG or EKG) which is a graphic recordation of cardiac activity, either on paper or a computer monitor. An ECG can be beneficial in detecting disease and/or damage; (iii) echocardiogram (heart ultrasound) used to investigate congenital heart disease and assessing abnormalities of the heart wall, including functional abnormalities of the heart wall, valves and blood vessels; (iv) Doppler ultrasound can be used to measure blood flow across a heart valve; (v) nuclear medicine imaging (also referred to as radionuclide scanning in the art) allows visualization of the anatomy and function of an organ, and can be used to detect coronary artery disease, myocardial infarction, valve disease, heart transplant rejection, check the effectiveness of bypass surgery, or to select patients for angioplasty or coronary bypass graft.

In some embodiments in this aspect and all other aspects described herein, subjects amenable to the pharmaceutical compositions as disclosed herein is a subject identified to be at risk of a large myocardial infarction. In some embodiments, a subject has been identified to have nucleic acid variances in the coding regions and non-coding regions of the A₁-AR, A_{2A}-AR or A₃-AR genes, for example as disclosed in U.S. Provisional Patent application 60/857,562 and PCT/US2007/684083 which are incorporated herein in their entirety by reference. Accordingly, a subject identified to have a likelihood of a higher risk of a large infarction using the methods as disclosed in U.S. Provisional Patent application 60/857,562 or PCT/US2007/684083 is a suitable subject amenable to administration of the pharmaceutical compositions comprising agents which result in the activation of both A₁-AR and A₂-AR.

In some embodiments in this aspect and all other aspects described herein, subjects amenable to the pharmaceutical compositions as disclosed herein is a subject identified to be presently on adenosine treatment or adenosine therapy, for example a subject on any treatment that acts as adenosine, adenosine analogues and mimetics and variants thereof, adenosine receptor agonists, selective adenosine agonists and dual activating adenosine agonists and variants and analogues thereof.

In some embodiments in this aspect and all other aspects described herein, adenosine treatment can include prophylaxis, including agents which slow or prevent the infarction. In other embodiments, adenosine treatment is any means to activate the adenosine pathway and/or adenosine receptors. In some embodiments, adenosine treatment is an adenosine or adenosine analogue, for example orally available adenosine analogues, or injectable form of adenosine, such as ADENOSCAN®. In some embodiments, adenosine treatment is any means to activate the adenosine pathway and/or adenosine receptors. In some embodiments, adenosine treatment is an adenosine or adenosine analogue, for example orally available adenosine analogues. In other embodiments, adenosine treatment is an adenosine receptor agonist. For example, an adenosine receptor agonist can be an A₁-AR selective agonist or a A_{2A}-AR selective agonist or a A₃-AR selective agonist.

As used herein, the term “adenosine therapy” or “adenosine receptor agonists” are used interchangeably herein broadly refers to use of any treatment that acts as adenosine, adenosine analogues and mimetics and variants thereof, adenosine receptor agonists, selective adenosine agonists and dual activating adenosine agonists and variants and analogues thereof. Adenosine receptor agonists are also intended to refer to treatment that increase endogenous adenosine levels and/or increase the expression of the A₁-adenosine receptor and/or A₃-AR.

It is a further object of the invention to provide a kit for providing cardioprotection in a subject, the kit comprising a pharmaceutical composition comprising a pharmaceutically acceptable carrier and pharmaceutically effective amounts of an A₁ adenosine receptor agonist and an A₂ adenosine receptor agonists and/or a dual agonist of A₁/A₂ adenosine receptors.

### Administration of Pharmaceutical Compositions

The pharmaceutical compositions as disclosed herein can be prepared according to any method known by persons of ordinary skill in the art, such as customary methods, using one or more pharmaceutically acceptable carrier, which comprise adjuvants or excipients. In some embodiments and adjuvant can comprise, inter alia, diluents, sterile aqueous media and the various non-toxic organic solvents. The compositions may be presented in the form of tablets, pills, capsules, lozenges, troches, hard candies, granules, powders, aqueous solutions or suspensions, injectable solutions, elixirs or syrups, powders, solution or suspension for intrapulmonary administration and can contain one or more agents chosen from the group comprising sweeteners, flavorings, colorings, or stabilizers in order to obtain pharmaceutically acceptable preparations.

The pharmaceutical compositions as disclosed herein can comprise agents or pharmaceutical acceptable carriers or vehicles which are suitable for the A₁-AR and/or A_{2A}-AR or A₁/A_{2A}-AR agonist agents in accordance with the solubility and chemical properties of such A₁-AR and/or A_{2A}-AR or A₁/A_{2A}-AR agonist, the particular mode of administration and the provisions to be observed in pharmaceutical practice. For example, excipients such as sterile water, Ringer's solution, lactose, sodium citrate, isotonic saline solutions (monosodium or disodium phosphate, sodium, potassium, calcium or magnesium chloride, or mixtures of such salts), calcium carbonate and disintegrating agents such as starch, alginic acids and certain complex silicates combined with lubricants such as magnesium stearate, sodium lauryl sulfate and talc may be used for preparing tablets. To prepare a capsule, it is advantageous to use lactose and high molecular weight polyethylene glycols. When aqueous suspensions are used they can contain emulsifying agents or agents which facilitate suspension. Diluents such as sucrose, ethanol, polyethylene glycol, propylene glycol, glycerol and chloroform or mixtures thereof may also be used.

In some embodiments, the pharmaceutical composition as disclosed herein can be administered parenterally, topically, rectally, transdermally, intrapulmonary or orally. In some embodiments, administration is parenterally and/or orally. Suitable pharmaceutical compositions can further comprise pharmaceutically acceptable carriers and can be prepared by any conventional means known to persons of ordinary skill in the art. For example, the compounds used according to the invention may be dissolved or suspended in a suitable carrier.

In some embodiments, the pharmaceutical compositions as disclosed herein can be presented in forms permitting administration by the most suitable route. In some embodiments, the pharmaceutical compositions as disclosed herein are suitable for use in human or veterinary medicine.

For parenteral administration, emulsions, suspensions or solutions of the compounds used according to the invention in vegetable oil, for example sesame oil, groundnut oil or olive oil, or aqueous-organic solutions such as water and propylene glycol, injectable organic esters such as ethyl oleate, as well as sterile aqueous solutions of the pharmaceutically acceptable salts, are useful. The solutions of the salts of the compounds used according to the invention are especially useful for administration by intramuscular, intravenous, intraarterial or subcutaneous injection or infusion techniques. The aqueous solutions, also comprising solutions of the salts in pure distilled water, may be used for intravenous administration with the proviso that their pH is suitably adjusted, that they are judiciously buffered and rendered isotonic with a sufficient quantity of glucose or sodium chloride and that they are sterilized by heating, irradiation or microfiltration.

In some embodiments, a pharmaceutical composition useful in the methods as disclosed herein can be formulated in a manner which resists rapid clearance from the vascular (arterial or venous) wall by convection and/or diffusion, thereby increasing the residence time of the composition at the desired site of action. In some embodiments, the pharmaceutical composition as disclosed herein can be in the form or a depot or depository, or in a capsule, for example in a copolymer matrix, such as ethylene-vinyl acetate, or a polyvinyl alcohol gel surrounded by a Silastic shell. Alternatively, in some embodiments, agents activating A₁ adenosine receptor and/or A₂ adenosine receptor can be administered in a form that they are administered simultaneously or separately, for example administered sequentially, for example, as such local delivery from a silicone polymer implanted in the adventitia.

In some embodiments, the pharmaceutical composition as disclosed herein can further comprise agents to extend the half life of agents which simultaneously activate A₁-AR and A_{2A}-AR. One approach that can be used to minimize the half-life of an agent or agents that activate A₁-AR and A_{2A}-AR, either simultaneously or separately, (i.e. for reducing the half life during percutaneous, transvascular delivery of such a pharmaceutical composition) comprises the use of nondiffusible, drug-eluting microparticles. The microparticles can be comprised of a variety of synthetic polymers, such as polylactide for example, or natural substances, including proteins or polysaccharides. Such microparticles enable strategic manipulation of variables including total dose of a drug and kinetics of its release. Microparticles can be injected efficiently into the arterial or venous wall through a porous balloon catheter or a balloon over stent, and are retained in the vascular wall and the periadventitial tissue for at least about two weeks. Formulations and methodologies for local, intravascular site-specific delivery of therapeutic agents are discussed, for example, in Reissen et al. (J. Am. Coll. Cardiol. 1994; 23: 1234-1244), the entire contents of which are hereby incorporated by reference.

In some embodiments, the pharmaceutical composition as disclosed herein which comprise at least one agent which dually activates the A₁ adenosine receptor and the A_{2A} adenosine receptors, either simultaneously or separately can further comprise a hydrogel. A hydrogel useful can be prepared from any biocompatible or non-cytotoxic (homo or hetero) polymer, such as a hydrophilic polyacrylic acid polymer that can act as a drug absorbing sponge. Such polymers have been described, for example, in application WO93/08845, the entire contents of which are hereby incorporated by reference. Certain of them, such as, in particular, those obtained from ethylene and/or propylene oxide are commercially available.

In further embodiments, the pharmaceutical composition as disclosed herein comprising agents for dual activation of A₁ adenosine receptor and A_{2A} adenosine receptor, either simultaneously or separately can be administered directly to the blood vessel wall by means of an angioplasty balloon which is coated with a hydrophilic film (for example a hydrogel), or by means of any other catheter containing an infusion chamber for the compounds, which can thus be applied in a precise manner to the site to be treated.

In some embodiments, the pharmaceutical composition comprises at least one agent which activate A₁-AR and at least one agent that activates A_{2A}-AR in a stoichiometric relationship, for example, an agent useful in the methods as disclosed herein activates A₁-AR and A_{2A}-AR with a 1:1 ratio, or a ratio of between 1:1-2, for example a ratio of about a 1:1.125 ratio, or about a 1:1.25 ratio, or about a 1:1.5 ratio, or a about 1:1.75 ratio or about 1:2 ratio. In alternative embodiments, the pharmaceutical composition can comprise at least one agent which activates the A_{2A}-AR and at least one agent that activates A₁-AR in a stoichiometric relationship of a ratio of between 1:1-2, for example a ratio of 1:1 or a ration of about a 1:1.125, or about a 1:1.25, or about 1:5, or about 1:1.75 or a about 1:2, and varying rations between. Optionally, the pharmaceutical composition further comprises a suitable amount of one or more β-blockers.

As such, the pharmaceutical composition as disclosed herein comprises effective amount of an agent or agents which activate both A₁-AR and activate A_{2A}-AR in a biologically matched manner, so that each receptor is activated to an equal extent. For example and as an illustrative example only, if the pharmaceutical composition comprises an agent which increases the activity of the A₁-AR by two fold, the composition can also comprise at an effective amount of least one agent or the sum of several agents which acting together result in an increase in the activation of the A_{2A}-AR by about two fold. For example, if the pharmaceutical composition comprises an agent which increases the biological activation of the A₁-AR by two fold, the composition also comprises at an effective amount of least one agent or the sum of several agents which acting together result in an increase in the biological activity of the A_{2A}-AR within 10% of the level of the biological activation of A₁-AR, i.e. A_{2A}-AR is activated by two fold ±10%. Typically, the level of A₁-adenosine receptor biological activation is measured by activation of Gi-protein, and the level of the A_{2A}-adenosine receptor biological activation is measured by activation of Gₛ-protein.

In the adult, the dosages of pharmaceutical composition comprising an agent or agents that activate A₁ adenosine receptor and A_{2A} adenosine receptor, either simultaneously or separately, or optionally in combination with one or more β-blockers, are generally from about 0.00001 to about 0.5, preferably about 0.0001 to about 0.05, mg/kg body weight per day by inhalation, from about 0.0001 to about 1, preferably 0.001 to 0.5, mg/kg body weight per day by oral administration, and from about 0.00001 to about 0.1, preferably 0.0001 to 0.01, mg/kg body weight per day by intravenous administration. The agent or agents that activate A₁ adenosine receptor and A_{2A} adenosine receptor, either simultaneously or separately may be administered in dosages which are pharmaceutically effective for each compound, or in dosages which are sub-clinical, i.e., less than pharmaceutically effective for each, or a combination thereof, provided that the combined dosages are pharmaceutically effective. In some embodiments, where a β-blocker is included in the pharmaceutical composition, an effective amount or dose of a β-blocker used is the amount sufficient to block activity of beta-adrenergic receptor or the action of endogenous catecholamine and norepinephrine on β-adrenergic receptors. In alternative embodiments, the dose of a β-blocker used is an amount less than the amount required to block activity of beta-adrenergic receptor or the action of endogenous catecholamine and norepinephrine on β-adrenergic receptors.

In this aspect and all other aspects described herein, an agent or agents that activate the A₁ adenosine receptor and the A_{2A} adenosine receptor, either simultaneously or separately used according to the invention may be administered as frequently as necessary in order to obtain the desired therapeutic effect. The dosage regimen in carrying out the method of this invention is that which insures maximum therapeutic response until improvement is obtained and thereafter the minimum effective level which gives relief. Some patients may respond rapidly to a higher or lower dose and may find much lower maintenance doses adequate. Both short- and long-term treatments regimens are contemplated for the invention. Treatments at the rate of about 1 to about 4 doses per day are also contemplated, in accordance with the physiological requirements of each particular patient, bearing in mind, of course, that in selecting the appropriate dosages in any specific case, consideration must be given to the patient's weight, general health, age, and other factors which may influence response to the drug. Continuous parenteral infusion, in order to maintain therapeutically effective blood levels of the pharmaceutical composition comprising an agent that activate A₁ adenosine receptor and A_{2A} adenosine receptor, either simultaneously or separately is also contemplated.

In this aspect and all other aspects described herein, an agent or agents that activate A₁ adenosine receptor and A_{2A} adenosine receptor, either simultaneously or separately as described herein can be used during the treatment of restenosis during angioplasty using any device such as balloon, ablation or laser techniques, in order to reduce or protect against injury during reperfusion.

In this aspect and all other aspects described herein, an agent or agents that activate A₁ adenosine receptor and A_{2A} adenosine receptor, either simultaneously or separately as described herein can used during the treatment of restenosis, in order to reduce or protect against injury during reperfusion, in combination with any anticoagulant, antiplatelet, antithrombotic or profibrinolytic agent. Often patients are concurrently treated prior, during and after interventional procedures with agents of these classes either in order to safely perform the interventional procedure or to prevent deleterious effects of thrombus formation. Some examples of classes of agents known to be anticoagulant, antiplatelet, antithrombotic or profibrinolytic agents include any formulation of thrombin inhibitors or Factor VIIa inhibitors. Some examples of classes of agents known to be anticoagulant, antiplatelet, antithrombotic or profibrinolytic agents include any formulation of aspirin, direct thrombin inhibitors, direct Factor Xa inhibitors, or Factor VIIa inhibitors.

Screening for Agents that Co-Activate A₁ and A₂ Adenosine Receptors

Another aspect of the present invention relates to a method to identify potential agents for treating and/or preventing and/or reducing the risk of developing diseases of the cardiovascular system in a subject. In some embodiments, the subject is a human or non-human animal. Another aspect of the present invention pertains to a screening method, wherein cells can be induced to overexpressing A₁ adenosine receptor is used as a biological model for searching for agents active against heart failure. In some embodiments, the cell is derived from a transgenic animal. In some embodiments, the transgenic animal is a transgenic mouse

Another aspect of the present invention relates to a method to identify agents which function as co-agonists of A₁-AR and A_{2A}-AR for the treatment and/or prevention and/or to reduce the risk of developing diseases of the cardiovascular system in a subject. In some embodiments of this aspect and all other aspects described herein, the subject is a human or non-human animal. Another aspect of the present invention pertains to a screening method, wherein a cells can be induced to overexpressing A₁ adenosine receptor is used as a biological model for searching for agents active against heart failure. In some embodiments, the cell is derived from a transgenic animal. In some embodiments, the transgenic animal is a transgenic mouse.

Several screening methods for compounds preventing or treating cardiovascular diseases are already known in the art. In this respect, Numann et al. describe a method, wherein compounds may be tested in cell cultures with respect to their ion channel binding properties (Numann and Negulescu, Trends Cardiovasc. Med. 11:54-59 (2001)). According to another method, compounds are tested on isolated and perfused hearts (R. Bessho, D. J. J. Chambers, Thorac Cardiovasc. Surg. 122:993-1003 (2001)). In addition thereto, in vivo testing methods are known, wherein the effect of compounds on the cardiovascular system is monitored by the means of electrocardiography, magnetic resonance imaging, or echocardiography in living animals (Chu et al., BMC Physiol 1:6-11 (2001); Krupnick et al., J Heart Lung Transplant 21:233-43 (2002)).

Use of AMP 579 in Combination with Aldose Reductase Inhibitors (ARIs)

Another aspect of the present the invention relates to a pharmaceutical composition comprising AMP 579 disclosed herein and aldose reductase inhibitors (ARIs). ARIs are an experimental class of medications that inhibit an enzyme (protein that produces chemical reactions in the body) called aldose reductase. Aldose reductase is normally present in many other parts of the body, and catalyzes one of the steps in the sorbitol (polyol) pathway that is responsible for fructose formation from glucose. It normally increases the rate at which aldoses (types of sugars) are reduced to sorbitol, a sugar alcohol. Sorbitol can cause problems for people with diabetes, who are vulnerable to high glucose (blood sugar).

Another aspect of the present invention provides method of treating cardiovascular diseases comprising administering to the subject a pharmaceutical composition comprising an effective amount of an effective amount of AMP 579 and aldose reductase inhibitor.

There are may known aldose reductase inhibitors that can be used in combination with AMP 579 according to the present invention, such as those disclosed in U.S. Pat. No. 7,144,900, herein incorporated by reference in its entirety. Examples of the aldose reductase inhibitors include tolurestat; epalrestat; 3,4-dihydro-2,8-diisopropyl-3-thioxo-2H-1,4-benzoxazine-4-acetic acid; 2,7-difluoro-spiro(9H-fluorene-9,4′-imidazolidine)-2′,5′-dione (generic name: imirestat); 3-[(4-bromo-2-fluorophenyl)methyl]-7-chloro-3,4-dihydro-2,4-dioxo-1(2H)-q-uinazoline acetic acid (generic name: zenarestat); 6-fluoro-2,3-dihydro-2′,5′-dioxo-spiro[4H-1-benzopyran-4,4′-imidazolidine]-2-carboxamide (SNK-860); zopolrestat; sorbini]; 1-[(3-bromo-2-benzofuranyl)sulfonyl]-2,4-imidazolidinedione (M-16209), etc.

In some embodiments of the present invention may be defined in any of the following numbered paragraphs:

1. A method for treating or preventing cardiac dysfunction in a subject having, or at risk of having cardiac dysfunction, the method comprising administering to the subject a pharmaceutical composition comprising, or alternatively consisting essentially of, or alternatively consisting of, an effective amount of at least one agent which co-activates both an A₁-adenosine receptor (A₁-AR) and an A_{2A}-adenosine receptor (A_{2A}-AR), or a combination of at least one agent which activates an A₁-adenosine receptor (A₁-AR) and at least one agent which activates an A_{2A}-adenosine receptor (A_{2A}-AR), wherein the pharmaceutical composition results in a level of biological activation of the A₁-adenosine receptor is within about 10% of the level of biological activation of the A_{2A}-adenosine receptor, wherein the level of the A₁-adenosine receptor biological activation is measured by detecting activation of Gi-protein, and the level of the A_{2A}-adenosine receptor is measured by detecting activation of Gₛ-protein.
2. A method for treating or preventing cardiac dysfunction in a subject having, or at risk of having cardiac dysfunction, the method comprising administering to the subject a pharmaceutical composition comprising, or alternatively consisting essentially of, or alternatively consisting of, an effective amount of at least one agent which co-activates both an A₁-adenosine receptor (A₁-AR) and an A_{2A}-adenosine receptor (A_{2A}-AR), or a combination of at least one agent which activates an A₁-adenosine receptor (A₁-AR) and at least one agent which activates an A_{2A}-adenosine receptor (A_{2A}-AR), wherein the at least one agent that co-activates the A₁-adenosine receptor and the A_{2A}-adenosine receptors, or the at least one agent that activates the A₁-adenosine receptor has a lower Kᵢ as compared to Kᵢ of at least one agent which activates the A_{2A}-adenosine receptor.
3. A method for treating or preventing a subject having or at risk of having cardiac dysfunction comprising administering to the subject a pharmaceutical composition comprising, or alternatively consisting essentially of, or alternatively consisting of, an effective amount of a combination of at least one agent which activates an A₁-adenosine receptor (A₁-AR) and at least one agent which activates an A_{2A}-adenosine receptor (A_{2A}-AR), wherein the pharmaceutical composition comprises at least a 1.5 fold higher amount of the at least one agent which activates the A₁-adenosine receptor as compared to the amount of the at least one agent which activates the A_{2A}-adenosine receptor activation.
4. A method for enhancing cardiac function in a subject comprising;
(a) selecting a subject in need of, or currently being treated an adenosine agonist therapy;
(b) administering to the subject a pharmaceutical composition comprising, or alternatively consisting essentially of, or alternatively consisting of, at least one agent which co-activates both an A₁-adenosine receptor (A₁-AR) and an A_{2A}-adenosine receptor (A_{2A}-AR), or a combination of at least one agent which activates an A₁-adenosine receptor (A₁-AR) and at least one agent which activates an A_{2A}-adenosine receptor (A_{2A}-AR), wherein the level of activation of A₁-AR is about the same as the level of activation of A_{2A}-AR.
5. The method of any of paragraphs 1, 2, 3 or 4, wherein the subject is first diagnosed as having, or at risk of having a cardiac dysfunction, wherein a subject identified as having, or at risk of having a cardiac dysfunction is then treated for cardiac dysfunction according to the methods of paragraphs 1, 2, 3 or 4.
6. The method of any of paragraphs 1, 2, 3 or 4, wherein the pharmaceutical composition is free of a sodium-hydrogen exchanger inhibitory compound.
7. The method of any of paragraphs 1, 2, 3 or 4, wherein the subject in need is at risk of having or has had myocardial infarction.
8. The method of any of paragraphs 1, 2, 3 or 4, wherein the subject in need is a subject with chronic heart failure.
9. The method of paragraph 8, wherein the subject with chronic heart failure has chronic or acute myocardial ischemia and reperfusion injury, cardiomyopathy, myocarditis, cardiac hypertrophy, ventricular remodeling, coronary ischemia or congestive heart failure.
10. The method of any of paragraphs 1, 2, 3 or 4 or 8, wherein the subject is undergoing coronary intervention.
11. The method of paragraph 10, wherein the subject is undergoing percutaneous coronary intervention.
12. The method of any of paragraphs 1, 2, 3 or 4, wherein the subject is prior to or undergoing or post surgery having a potential to cause cardiac ischemic damage.
13. The method of paragraph 10, wherein the subject is prior to, or undergoing or post surgery having cardiac surgery.
14. The method of any of paragraphs 1, 2, 3 or 4, wherein at least one agent is selected from the group consisting of: a small molecule, a nucleic acid, a nucleic acid analogue, an aptamer, a ribosome, a peptide, a protein, an avimer, an antibody, an siRNA, a miRNA, an shRNA, PNA, pc-PNA or variants or pharmaceutical salts and fragments thereof.
15. The method of any of paragraphs 1, 2, 3, 4 or 14, wherein the agent which activates both an A₁-adenosine receptor and an A_{2A}-adenosine receptor is AMP579 or a derivative thereof.
16. The method of any of paragraphs 1, 2, 3, 4 or 14, wherein the agent which activates both an A₁-adenosine receptor (A₁-AR) and an A_{2A}-adenosine receptor (A_{2A}-AR) is a binary conjugate of at least one agent which activates A₁-AR and at least one agent which activates A_{2A}-AR.
17. A pharmaceutical composition comprising, or alternatively consisting essentially of, or alternatively consisting of, a combination of at least one agent which activates an A₁-adenosine receptor and at least one agent which activates an A_{2A}-adenosine receptor.
18. A pharmaceutical composition comprising, or alternatively consisting essentially of, or alternatively consisting of, at least one agent which co-activates both an A₁-adenosine receptor (A₁-AR) and an A_{2A}-adenosine receptor (A_{2A}-AR) and a pharmaceutically acceptable carrier.
19. The pharmaceutical composition of paragraph 17, wherein the combination of at least one agent which activates an A₁-adenosine receptor and at least one agent which activates an A_{2A}-adenosine receptor results in substantially the same level of biological activation of both the A₁-adenosine receptor and the A_{2A}-adenosine receptor.
20. The pharmaceutical composition of paragraph 18, wherein the agent which co-activates both an A₁-adenosine receptor (A₁-AR) and an A_{2A}-adenosine receptor (A_{2A}-AR) results in substantially the same level of biological activation of both the A₁-adenosine receptor and the A_{2A}-adenosine receptor.
21. The pharmaceutical composition of paragraph 18, wherein the agent which co-activates both an A₁-adenosine receptor (A₁-AR) and an A_{2A}-adenosine receptor (A_{2A}-AR) is at least one agent which activates the A₁-adenosine receptor conjugated to at least one agent which activates the A_{2A}-adenosine receptor.
22. The pharmaceutical composition of paragraphs 17 or 21, wherein the at least one agent which activates A₁-AR is selected from the group consisting of; AB-MECA, CPA, ADAC, CCPA, CHA, GR79236, S-ENBA, IAB-MECA, R-PIA, ATL146e, CGS-21680, CV1808, NECA, PAPA-APEC, DITC APEC, DPMA, S-PHPNECA, WRC-0470, IB-MECA, 2-CIADO, I-ABA, S-PIA, C1-IB MECA, polyadenylic acid or pharmaceutically acceptable analogues or derivatives or salts thereof.
23. The pharmaceutical composition of paragraphs 17 or 21, wherein the at least one agent which activates A_{2A}-AR is selected from the group consisting of; 2-cyclohexylmethylenehydrazinoadenosine, 2-(3-cyclohexenyl)methylenehydrazinoadenosine, 2-isopropylmethylenehydrazinoadenosine, N-ethyl-1′-deoxy-1′-[6-amino-2-[(2-thiazolyl)ethynyl]-9H-purin-9-yl]-β-D-ribofuranuronamide, N-ethyl-1′-deoxy-1′-[6-amino-2-[hexynyl]-9H-purin-9-yl]-β-D-ribofuranuronamide, 2-(1-hexyn-1-yl)adenosine 5′-N-methyluronamide, 5′-chloro-5′-deoxy-2-(1-hexyn-1-yl)adenosine, N₆-[2-[(3,5-dimethoxyphenyl)-2-(2-methylphenyl)adenosine, 2-(2-phenyl)ethoxyadenosine, 2-[2-(4-methylphenyl)ethoxy]adenosine, 2-[2-(4-fluorophenyl)ethoxy]adenosine, 2-(2-(2-naphthyl)ethoxy)adenosine, 2-[p-(2-carboxyethyl)phenethylamino-5′-N-ethylcarboxamidoadenosine (CGS-21680), 2-(2-cyclohexyl)ethoxyadenosine, 2-octynyladenosine (YT-146), 2-thiazolylethynyladenosine and 2-phenethylamino-5′-N-ethylcarboxamidoadenosine (CGS-21577) or pharmaceutically acceptable analogues or derivatives or salts thereof.
24. Use of the pharmaceutical composition of paragraphs 17 or 18 for the treatment or prevention of myocardial infarction in a subject.
25. Use of the pharmaceutical composition of paragraphs 17 or 18 for the treatment or prevention of chronic heart failure in a subject.
26. Use of the pharmaceutical composition of paragraphs 17 or 18 for the treatment or prevention of chronic or acute myocardial ischemia and reperfusion injury, cardiomyopathy, myocarditis, cardiac hypertrophy, ventricular remodeling, coronary ischemia or congestive heart failure in a subject.
27. A pharmaceutical composition comprising, or alternatively consisting essentially of, or alternatively consisting of, an effective amount of AMP 579 or pharmaceutically acceptable analogues or derivatives or salts thereof, and aldose reductase inhibitor.
28. The pharmaceutical composition according to paragraph 27, wherein the aldose reductase inhibitor is selected from the group consisting of: epalrestat; 3,4-dihydro-2,8-diisopropyl-3-thioxo-2H-1,4-benzoxazine-4-acetic acid; 2,7-difluoro-spiro(9H-fluorene-9,4′-imidazolidine)-2′,5′-dione; 3-[(4-bromo-2-fluorophenyl)methyl]-7-chloro-3,4-dihydro-2,4-dioxo-1(2H)-q-uinazoline acetic acid; 6-fluoro-2,3-dihydro-2′,5′-dioxo-spiro[4H-1-benzopyran-4,4′-imidazolidine-]-2-carboxamide; zopolrestat; sorbini; and 1-[(3-bromo-2-benzofuranyl)sulfonyl]-2,4-imidazolidinedione.
29. A method for treating or preventing cardiac dysfunction in a subject having, or at risk of having cardiac dysfunction, the method comprising administering to the subject a pharmaceutical composition comprising, or alternatively consisting essentially of, or alternatively consisting of, an effective amount of an AMP 579 and an aldose reductase inhibitor.
30. A method for treating or preventing cardiac dysfunction in a subject having, or at risk of having cardiac dysfunction, the method comprising administering to the subject a pharmaceutical composition comprising, or alternatively consisting essentially of, or alternatively consisting of, an effective amount of an AMP 579 and a β-blocker.
27. The method of any of paragraphs 1, 2, 3 or 4, wherein the pharmaceutical composition comprises a β-blocker.
28. The method of any of paragraphs 1, 2, 3 or 4, wherein the pharmaceutical composition comprises an aldose reductase inhibitor.
29. The pharmaceutical composition of any of paragraphs 17 or 18, wherein the pharmaceutical composition optionally comprises a β-blocker.
30. The pharmaceutical composition of any of paragraphs 17 or 18, wherein the pharmaceutical composition optionally comprises an aldose reductase inhibitor.

### EXAMPLES

The examples presented herein relate to the discovery that activation of A₁-AR alone, or activation of A_{2A}-AR alone result in compromised cardiac function, and that activation of A₁-AR and A_{2A}-AR both together (i.e. substantially simultaneously) ameliorates the compromised cardiac function which occurs when each adenosine receptor is the only receptor activated. Throughout this application, various publications are referenced. The disclosures of all of the publications and those references cited within those publications in their entireties are hereby incorporated by reference into this application in order to more fully describe the state of the art to which this invention pertains. The following examples are not intended to limit the scope of the claims to the invention, but are rather intended to be exemplary of certain embodiments. Any variations in the exemplified methods which occur to the skilled artisan are intended to fall within the scope of the present invention.

Methods

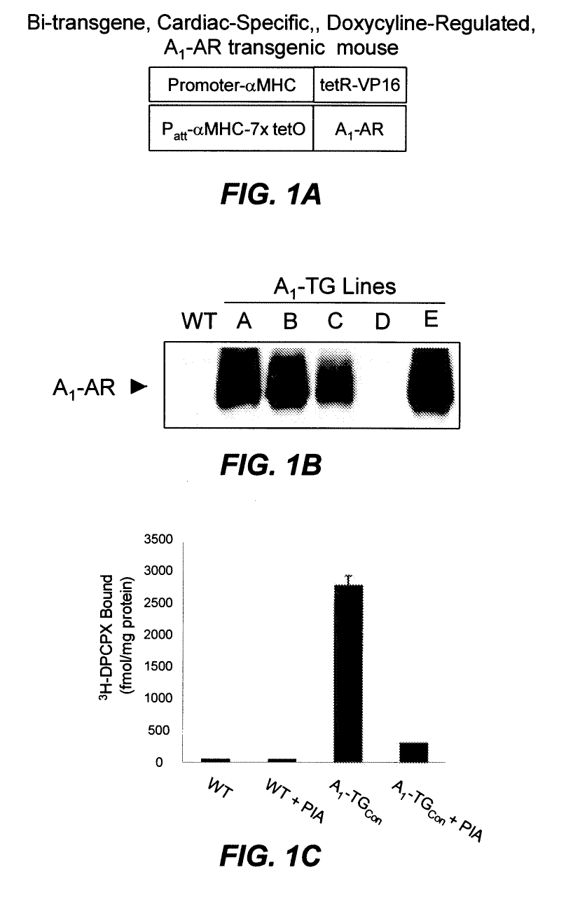

Transgenic mouse generation. The human A₁-AR cDNA was cloned into a cardiac-specific and inducible controlled vector (TREMHC)⁶⁴ composed of a modified mouse α-myosin heavy chain minimal promoter fused with nucleotide binding sites for tetracyline transactivating factor (tTA).³³ A₁-AR transgenic mice, engineered on a FVB background (PolyGene, Switzerland), were crossed with mice that expressed tTA in the heart (MHC-tTA) (FIG. 1A). In this “tet-off” inducible system, the stable tetracycline analog, doxycycline (DOX), inhibits tTA transactivation and was administered to mice at 300 mg/kg of mouse diet (Bio-Serv, NJ). Survival studies were performed using both male and female mice and found no statistical significant differences. Therefore, only male mice were used for subsequent studies. All protocols were approved by the Institutional Animal Care and Use Committee of Thomas Jefferson University.

Animal Model of TNFα. Experiments were carried out in transgenic mice with cardiac-restricted over expression of TNFα (TNF 1.6 mice)⁷⁷⁻⁷⁹. Non-transgenic litter served as controls and unless otherwise noted, all mice were male. The TNF 1.6 mice were engineered on an FVB background. Studies were also performed in two additional murine heart failure models: mice overexpressing calsequestrin^{80,81} in DBA/2 background and C57BL/6 mice who underwent chronic aortic banding. TNF 1.6 mice were crossed with mice in which either the TNF\acute{α} Receptor 1 (TNFR1) or Receptor 2 (TNFR2) had been ablated as previously described.⁸² All protocols were approved by the Institutional Animal Care and Use Committee of Thomas Jefferson University.

Echocardiography and Electrocardiogram (ECG). Echocardiographic studies on A₁-AR transgenic mice were performed using an ultrasonographic system (ACUSON Sequoia C256) as described.^{34,35} Briefly Echocardiographic studies on A₁-AR transgenic mice were performed using an ultrasonographic system (ACUSON Sequoia C256) as described.^{2,3} Non-transgenic littermates served as controls and unless otherwise noted, all mice were male. Mice were anesthetized with 2.5% Avertin (10 μl/g body weight, IP, Aldrich Chemical Co) and placed in the supine position. A 14-MHz transducer was applied to the left hemithorax. Two-dimensional targeted M-mode imaging was obtained from the short-axis view at the level of the greatest left ventricular dimension at baseline. M-mode measurements of left ventricular end-diastolic and end-systolic diameter and left ventricular anterior- and posterior-wall thickness were made using the leading-edge convention of the American Society of Echocardiography. End diastole was determined at the maximal left ventricular diastolic dimension, and end systole was taken at the peak of posterior-wall motion. To measure conscious heart rate, ECG recordings were obtained using PowerLab (ML866) and Bio amp (ML136) (Adinstruments). Briefly, animals were anaesthetized with inhalation of Isoflurane (Veterinary Supply, Inc), placed in a supine position and restrained. Mice returned to consciousness within 2 minutes. ECG was recorded for 2 minutes and analyzed using Char 5 software (Adinstruments). To normalize the recordings, mice were trained 3 times a day for 7 days before measurement.

Left Ventricular Hemodynamics Measurement. After anesthetization with 2.5% Avertin (10 μl/g body weight, IP, Aldrich Chemical Co), mice were placed in the supine position. A 1.4 F micromanometer catheter (Millar Instruments) was inserted into the left ventricle through the right carotid artery.^{64,65} Left ventricular pressure and heart rate were then recorded at baseline and 10 minutes after injection of CPA (0.1 mg/kg body weight, IP, Sigma-Aldrich Co).

Mouse Langendorff heart perfusion. As was previously described³⁶⁻³⁸, the inventors anesthetized mice were sacrificed with cervical dislocation. The abdominal cavity was immediately opened and the heart cooled with the ice-cold perfusion fluid. The aorta was cannulated above the aortic valve and retrograde perfusion was begun with a modified Krebs-Henseleit bicarbonate buffer, pH 7.4, equilibrated with 95% O₂/5% CO₂ at 37° C. Buffer composition was 113.8 mM NaCl, 22 mM NaHCO₃, 4.7 mM KCl, 1.2 mM KH₂PO₄, 1.1 mM MgSO4, 2.0 mM CaCl₂ and 11.0 mM glucose. The hearts were perfused using a Langendorff apparatus and paced at 400 beats per minute with a Grass stimulator (9 V, 0.5 ms, Grass Instruments, Quincey, Mass., USA). All hearts were immersed in a water-jacketed organ chamber to maintain a temperature of 37° C. A constant pressure protocol was used to compare the acute response between wild-type and A₁-TG mouse hearts. For hemodynamic measurements, a balloon was inserted into the left ventricle (LV) and the balloon volume was adjusted to 8-11 mmHg of LV end-diastolic pressure (LVEDP) for stabilization. Initially, each isolated heart was perfused with a constant pressure of 55 cm H₂O for 15 minutes. Then, the perfusion pressure was elevated to 120 cm H₂O for 45 minutes. Following stabilization, no further alterations in balloon volume were made. LV pressure (LVP), the maximum rate of positive and negative change in LVP (±dP/dt), and coronary perfusion pressures were continuously recorded (Powerlab/8SP, ADInstruments, Colorado Springs, Colo.). Coronary perfusion pressure was measured at heart level via a fluid-filled pressure transducer. At the end of the protocol, left and right ventricles were flash frozen with liquid nitrogen.

Real-Time Quantitative PCR. Real-time quantitative PCR analysis determined both genomic copies of inserted transgene and transgene expression using the human and mouse conserved A₁-AR primer sets; 5′-AAC ATT GGG CCA CAG ACC TAC TTC-3′ (SEQ ID NO: 1) and 5′-GAT GGA GCT CTG GGT GAG GAT GA-3′ (SEQ ID NO:2). These primers are 100% conserved between mouse and human A₁-AR. To quantify the number of transgenes inserted into the genome, genomic DNA from mouse tail was isolated using the Qiagen DNAeasy kit. Briefly, 40 ng of genomic DNA from mouse tail were used to quantify the number of transgenes inserted into the genome. To analyze the expression of A₁-AR transgene in the heart, total RNA was extracted from the bi-ventricular tissues and 10 μg total RNA was used to synthesize double-stranded cDNA with a SuperScript kit (InvitroGene), incorporating a T7 oligo(dT)24 (SEQ ID NO: 29) promoter primer. Reverse-transcribed cDNA from myocardium RNA were used to determine the expression of A₁-AR, ANP, SERCA, PLB, cFos, EGR-1, Collagen 1a, 3a and 6a genes using specific primer sets: ANP (5′ CGT GCC CCG ACC CAC GCC AGC ATG G 3′ (SEQ ID NO:3), 5′ GCC TCC GAG GGC CAG CGA GCA GAG C 3′ (SEQ ID NO:4)); PLB (5′ TAC CTC ACT CGC TCG GCT AT 3′ (SEQ ID NO:5), 5′ GAT GCA GAT CAG CAG CAG AC 3′ (SEQ ID NO:6)); SERCA₂ (5′ TGA GAC GCT CAA GTT TGT GG 3′(SEQ ID NO:7), 5′ ATG CAG AGG GCT GGT AGA TG 3′ (SEQ ID NO:8)); collagen 1a (5′ GCC TCA GAA GAA CTG GTA CAT CAG 3′ (SEQ ID NO:9), 5′ GGA AGG TCA GCT GGA TAG CGA CAT 3′ (SEQ ID NO:10)); collagen 3a (5′ GGA AAC AGA GGT GAA AGA GGA TCT 3′(SEQ ID NO. 11), 5′ TTT CAC CTC CAA CTC CAG CAA TGG 3′ (SEQ ID NO:12)); collagen 6a (5′ ATT GAC CGG TTG AGC AAG GAT GAG 3′ (SEQ ID NO:13); 5′ CTC TTG CAT CTG GTT GTG GCT GTA 3′ (SEQ ID NO:14)); A₁-AR (5′ AAC ATT GGG CCA CAG ACC TAC TTC 3′ (SEQ ID NO:15), 5′ GAT GGA GCT CTG GGT GAG GAT GA 3′ (SEQ ID NO:16)); cFos (5′ ATC GGC AGA AGG GGC AAA GTA G 3′ (SEQ ID NO:17), 5′ GCA ACG CAG ACT TCT CAT CTT CAA G-3′ (SEQ ID NO:18)); EGR-1 (5′ CCT TTT CTG ACA TCG CTC TGA A 3′ (SEQ ID NO:19), 5′ CGA GTC GTT TGG CTG GGA TA 3′ (SEQ ID NO:20)); actin (5′ AGG ACC TGT ACG CCA ACA AC 3′ (SEQ ID NO:21), 5′ACA TCT GCT GGA AGG TGG AC 3′ (SEQ ID NO:22)). Real time PCR was performed in a 50 μl reaction (5 μl cDNA or 40 ng of genomic DNA; 250 nM each primer; 1×SYBRE Green Master Mix). Each experimental group was performed in triplicate. The ΔCT method was used to quantify the results, which are presented as relative fold changes to the actin gene. Each primer set was designed assuming a Tm of 60° C. and 50% GC content.

Real-time quantitative PCR analysis was used to analyze specific gene expression changes in wild-type and TNF 1.6 mouse ventricles as described.³ Real time PCR was performed in 50 μl reaction (5 μl cDNA; 250 nM each primer; 1×SYBRE Green Master Mix). Three samples were measured in each experimental group in triplicate in a minimum of two independent experiments. ΔCT method was used for this study and the results were presented as relative fold changes of actin gene. Each primer set was designed for 60° C. of TM with 50% of GC content.

Membrane Preparation and A₁ Receptor Binding Assay. Radioligand binding of A₁-AR in crude cardiac membranes was performed as described.^{39,40} Radioligand binding of crude cardiac membranes was performed as described.^{4,5} Briefly, ventricular myocardium in ˜10 volumes of cold 50 mM Tris-HCl buffer pH 7.5 containing 2 mM EGTA, 250 mM sucrose and 1× protease inhibitor (Roche) was homogenized with a polytron homogenizer on ice. The resulting homogenate was centrifuged at 100×g for 10 min at 4° C. The supernatants were re-centrifuged at 14000×g for 12 min at 4° C. The pellets were then resuspended in a solution containing 50 mM Tris-HCl buffer pH 7.5, 250 mM Sucrose and 1 mM EGTA. Aliquots were frozen at −70° C.

A₁-AR binding was performed with 20-40 μg of membrane protein in 300 μL of incubation solution (50 mM Tris-HCl buffer pH 7.5, 2 mM MgCl₂ and 11 nM [³H]DPCPX) for 2 hr at 23-25° C. Nonspecific binding was measured in the presence of 100 uM R-PIA. All binding assays were performed in triplicate. The binding reactions were stopped by vacuum filtration. The washing volume was 10 mL cold 50 mM Tris-HCl buffer. The filters were transferred into scintillation vial containing 200 uL of 70% formic acid. The filter paper was soaked in the acid for at least a half hour before the scintillation liquid was added. The non-selective AR agonist, N₆-2-phenylisopropyl-adenosine (PIA), was from Sigma Chemical Company. Radio-labeled [³H]DPCPX was from GE Healthcare.

Immunoblotting and Histopathology of Myocardium. Immunoblotting of ventricular protein extracts was digitally detected using an Odyssey Infrared Imaging System as described.⁴¹ Briefly, frozen ventricular tissues were homogenized on ice using a non-ionic detergent-based lysis buffer (25 mM Tris-HCl pH 7.6, 137 mM NaCl, 10% glycerol, 1% NP40 or IGEPAL CA-630, 10 mM NaF) freshly supplemented with 1 mM Sodium pyrophosphate, 5 μg/ml leupeptin, 5 μg/ml aprotinin, 1 mM EDTA, 10 mM PMSF, and 1 mM NaVO4. After electrophoresis in a 4-12% SDS-PAGE and transfer onto nitrocellulose membranes, the blots were probed with the following antibodies (1:1000 dilution): anti-total Akt (Cell Signaling Tech.), anti-phosphoAkt (Thr308) (Cell Signaling Tech.), anti-actin (Sigma) and anti-GAPDH (Fitzgerald). The following antibodies were also used at 1:1000 dilution: anti-A₁ receptor (Affinity BioReagents), anti-A_{2A} receptor (Alpha Diagnostics), anti-ectonucleotide pyrophosphatase/phosphodiesterase 2 (Enpp2, Cayman Chem.), and anti-xanthine oxidase (XO, Lab-Vision) and anti-GAPDH (Fitzgerald). For immunoblotting overexpressed human A₁-AR with anti-A₁-AR antibodies (Affinity BioReagents), special precaution was taken to denature protein extract in the absence of reducing agent at 70° C. for 5 minutes. All blots were incubated with either IRDye 700 or 800 secondary antibodies and visualized using the Odyssey Infrared Imaging System software (Li-Cor, Lincoln, Nebr.).

Picro-sirius Red staining for assessment of fibrosis was performed by RADIL, U. of Missouri. To determine fibrosis, five independent high-power fields of stained images from each animal were analyzed using Image-Pro Plus Software.

Immunohistochemistry. The immunostaining with anti-A₁ AR antibody (ABR) was performed on the Dako Autostainer by MDR Global Systems (Windber, Pa.). Briefly, frozen sections of left ventricular myocardium were cut at 5 to 7 microns and placed on positively charged slides. The slides were allowed to dry at room temperature and then fixed in acetone. A peroxide procedure was used to block endogenous peroxidase. The primary antibody was applied to the slides and then detected by a non-avidin-biotin polymer peroxidase detection system. Diaminobenzidine “DAB”/hydrogen peroxide was used for color visualization. Once staining was completed, all the slides were counterstained with hematoxylin.

Enzyme-Linked Immunosorbent Assay. The protein levels were assessed using kits for mouse TNFα (Quantikine, R&D Systems) according to manufactures instructions as previously described.⁷ Results were expressed as picograms of target proteins per gram of tissue protein.

Surgical Procedure for Aortic Banding. 6-week-old male wild-type and transgenic FVB mice were used for aortic banding. Before the procedure, mice were anesthetized with 2.5% Avertin (10 μl/g body weight, IP), placed in a supine position, and ventilated with a tidal volume of 0.15 ml and a respiratory rate of 120 breaths per minute. A skin incision of 0.5-1.0 cm in length at the suprasternal notch and a 2- to 3-mm longitudinal incision at the proximal sternum allowed the visualization of the aortic arch under low-power magnification. Briefly, an aortic band was created by placing a ligature (7-0 nylon suture) securely between the origin of the right innominate and left common carotid arteries using a 27-gauge needle as a guide. After needle removal and skin closure, mice were allowed to recover on a warming pad until they were fully awake. The sham procedure was identical except that the aorta was not ligated. Echocardiography and heart harvest were performed 4 weeks after surgery.

Chemicals. The non-selective AR agonists, 2-chloroadenosine (CADO) and N⁶-2-phenylisopropyl [symbol] adenosine (PIA), the A₁-AR selective agonist, 2-chloro-N⁶-cyclopentanyladenosine (CPA) and the A_{2A} receptor selective agonist, 2-p-(2-carboxyethyl)phenethylamino-5′-N-ethylcaroxamino adenosine hydrochloride (CGS21680) were purchased from Sigma Chemical Company. Radio-labeled [³H]DPCPX were purchased from GE Healthcare.

Affymetrix Microarray Hybridization and Data Analyses. Affymetrix microarray analyses were performed using a standard protocol as described previously.⁶⁶ In brief, total RNA was extracted from the bi-ventricular tissues and 10 μg total RNA was used to synthesize double-stranded cDNA with a SuperScript kit (InvitroGene), incorporating a T7 oligo(dT)24 (SEQ ID NO: 29) promoter primer. Biotin-labeled cRNAs were then generated from the cDNA and hybridized to Affymetrix murine U74Av2 microarrays. RNA isolated from individual mice were hybridized on individual chips and each experimental grouping consisted of 3 chips. Data were analyzed with the Affymetrix GeneChip Operating Software (GCOS) and Affymetrix Data Mining Tool 2.0. Genes were considered significant if p-values were <0.05 for both statistical tests; signal intensity was >100. The analyses detailed here comply with MIAME (minimal information about a microarray experiment) guidelines established by the microarray gene expression data society (world-wide-web at mged.org) and the expression data for all samples described in this study can be obtained from Gene Expression Omnibus (GEO) web site (world-wide-web at: ncbi.nlm.nih.gov/geo/). Gene Ontology Mining Tool (Affymetrix web site) was used to define gene groups according to their function.

Cardiac ATP, ADP, AMP, Adenosine and Hypoxanthine Measurements. Mouse hearts were rapidly excised and frozen in liquid nitrogen to preserve adenosine. We compared the traditional Wollenberger clamp method^{8,9} to our modified rapid pinch-excision method. Both methods worked equally in preserving adenosine (data not shown). Briefly, under isoflurane anesthesia, beating hearts were exposed by opening the mouse chest cavity. The hearts were clamped with liquid nitrogen-cooled aluminum blocks or were rapidly and cleanly pinch-excised and immediately immersed in liquid nitrogen (within 1 sec). The pinch-excision method was more precise and avoided the scraping of sticky frozen tissue mass into plastic tubes. To extract adenosine, frozen heart tissues (20-25 mg) were rapidly boiled in 500 ul of water for 4 minutes (to inactivate adenosine deaminase and other enzymes). It should be noted that we avoided the acid-precipitation method for preserving adenosine because this method severely suppressed electro-spray ionization in the mass spectrometry source even after neutralization with base. Heat inactivation of tissue enzymes provided far more accurate and sensitive measurements of adenosine then did acid inactivation⁷³. The tissues were then homogenized with a power homogenizer. The homogenate was centrifuged at 14,0000 rpm for 5 minutes, and the supernatant centrifuged for a second time. The resulting supernatant was loaded onto centrifugal filter devices (Biomax-30, Millipore) and filtered to remove proteins. Aliquots were used for analysis. Adenosine, AMP, ADP, ATP and hypoxanthine were measured on a Thermofinnigan LCQ Duo mass spectrometer equipped with electrospray ionization as recently described for rat kidney.¹⁰

Adenosine, AMP, ADP, ATP and hypoxanthine were measured on a Thermofinnigan LCQ Duo mass spectrometer equipped with electrospray ionization as recently described for rat kidney⁷³. Briefly, analytes were resolved on a C-18 column with water methanol containing 7.5 mM N,N-dimethylhexylamine (ion pair agent) at a flow rate of 0.5 ml/min. The analytes were monitored using single ion monitoring in the positive ion mode: for AMP, mass-to-charge ratios (m/z)=477; for ADP m/z=557; and for ATP, m/z=766. For adenosine and hypoxanthine measurement, the filtrate was diluted 1:100 in water and internal standard (adenine 9-β-D arabinofuranoside) was added to a final concentration of 10 pg/ul. Standard curve was created in water and samples analyzed with an LCMS assay. The analytes were monitored using single ion monitoring: for adenosine and adenine 9-β-D arabinofuranoside (internal standard) the m/z was 268, and for hypoxanthine m/z was 137.

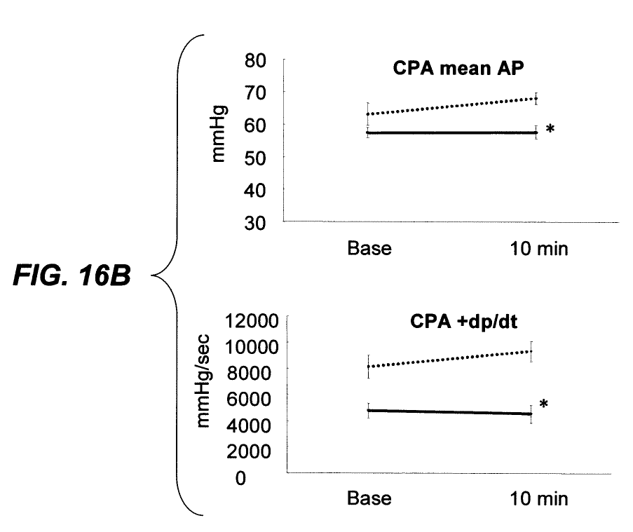

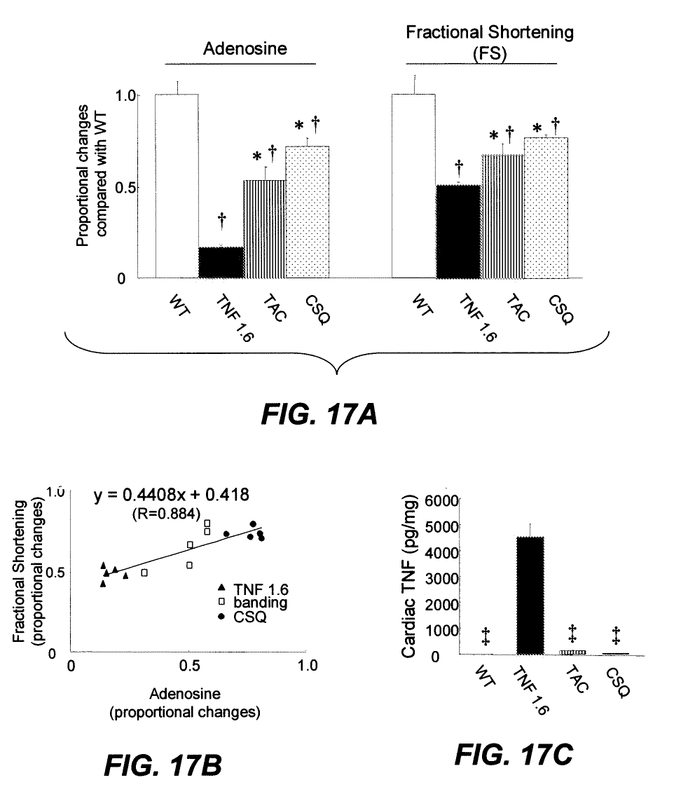

Statistical Analysis. Analysis was performed using SPSS for Windows (version 11.5). Kaplan-Meier survival curves were compared between group using log-rank tests. The results are presented as mean±SEM. One-way analysis of variance was performed using a Student-Newman-Keuls test. For multiple variables, we utilized a two-way analysis of variance. Categorical differences were analyzed using the Mann-Whitney test. Differences were considered to be statistically significant at P<0.05. In Example 5, statistical significance of in vivo cardiac responsiveness (the slope) between experimental value before drug administration and experimental value at 10 minutes after administration (FIGS. 16A and 16B) were compared using ANOVA General Linear Model with repeated measures. Positive correlation between adenosine levels and FS (FIG. 17B) was obtained using Linear Regression. All other data used non-parametric methods to protect against violation of ANOVA assumptions and two-tailed P value calculation. Differences were considered to be statistically significant at P<0.05.

Transgenic mouse generation. Experiments were carried out in transgenic mice with cardiac-restricted constitutive overexpression of the human A_{2A}-R engineered on an FVB background as previously described (127, 142). All protocols were approved by the Institutional Animal Care and Use Committee of Thomas Jefferson University.

Echocardiography. Echocardiographic studies were performed using an ultrasonographic system (ACUSON Sequoia C256) as described (143, 144). Age matching, non-transgenic mice in FVB background served as controls. Mice were anesthetized with 2.5% Avertin (10 μl/g body weight, IP, Aldrich Chemical Co) and placed in the supine position. A 14-MHz transducer was applied to the left hemithorax. Two-dimensional targeted M-mode imaging was obtained from the short-axis view at the level of the greatest left ventricular dimension at baseline. M-mode measurements of left ventricular end-diastolic and end-systolic diameter and left ventricular anterior- and posterior-wall thickness were made using the leading-edge convention of the American Society of Echocardiography. End diastole was determined at the maximal left ventricular diastolic dimension, and end systole was taken at the peak of posterior-wall motion.

Left ventricular hemodynamics measurement. After anesthetization with 2.5% Avertin (10 μl/g body weight, IP, Aldrich Chemical Co), mice were placed in the supine position. A 1.4 F micromanometer catheter (Millar Instruments) was inserted into the left ventricle through the right carotid artery (143, 144). Left ventricular pressure and heart rate were then recorded.

Immunoblotting and histopathology of myocardium. Frozen ventricular tissues were homogenized on ice using a non-ionic detergent-based lysis buffer (25 mM Tris-HCl pH 7.6, 137 mM NaCl, 10% glycerol, 1% NP40 or IGEPAL CA-630, 10 mM NaF) freshly supplemented with 1 mM Sodium pyrophosphate, 5 μg/ml leupeptin, 5 μg/ml aprotinin, 1 mM EDTA, 10 mM PMSF, and 1 mM NaVO4. After electrophoresis in SDS-PAGE and transfer onto nitrocellulose membranes, the blots were probed with anti-A₁-R (Affinity BioReagents), anti-A_{2A}-R (Millipore), anti-actin (Sigma), anti-Gαi (Abcam), anti-NCX1 (Swant), anti-SERCA₂ (Bethyl lab), anti-pT308-Akt (Cell Signaling), anti-total Akt (BD Biosciences), anti-calsequestrin (Swant) and anti-Na+-K+-ATPase (Gift from Dr. R. Levenson, Pennsylvania State University) as previously described (127, 145). For anti-A₁-R and anti-A_{2A}-R blots, special precaution was taken to denature protein extract in the absence of reducing agent at 70° C. for 5 minutes. All blots were incubated with either IRDye 700 or 800 secondary antibodies and visualized using the Odyssey Infrared Imaging System software (Li-Cor, Lincoln, Nebr.). Histopathology staining was performed by RADIL, U. of Missouri and imaged at TJU Pathology Imaging Facility.

Isolation of adult murine cardiac myocytes. Cardiac myocytes were isolated from the septum and left ventricular free wall of wild-type and transgenic mice (male, 8-9 week old) as recently describe (145). Briefly, mice were heparinized (1,500 U/kg ip) and anesthetized (pentobarbital sodium, 50 mg/kg ip). Excised heart was mounted on a steel cannula and retrograde perfused (100 cmH₂O, 37° C.) with Ca²⁺-free bicarbonate buffer followed by enzymatic digestion (collagenases B and D, protease XIV). Isolated myocytes were cultured on laminin-coated glass cover slips and the Ca²⁺ concentration of the buffer was progressively increased from 0.05 to 0.125 to 0.25 to 0.5 mM in three steps (10 min interval each). The 0.5 mM Ca²⁺ buffer was then aspirated and replaced with minimal essential medium (MEM, Sigma M1018) containing 1.2 mM Ca²⁺, 2.5% FBS, and antibiotics (1% penicillin/streptomycin). After 1 h (5% CO2, 37° C.), media was replaced with FBS-free MEM. Myocytes were used within 2-8 h of isolation.

Myocyte shortening measurements. Myocytes adherent to cover slips were bathed in 0.6 ml of air- and temperature-equilibrated (37° C.), HEPES-buffered (20 mM, pH 7.4) medium 199 containing 0.6, 1.8, or 5.0 mM [Ca₂+]o. Measurements of myocyte contraction (1 Hz) were performed using a charge-coupled device video camera and edge-detection software (Ionoptix, Milton, Mass.) as previously described (145-147).

[Ca²⁺]ᵢ transient measurements. Myocytes were exposed to 0.67 μM of fura-2 AM for 15 min at 37° C. Fura-2-loaded myocytes were field-stimulated to contract (1 Hz, 37° C.) in medium 199 containing 0.6, 1.8, or 5.0 mM [Ca²⁺]o. Fura-2 loaded myocytes mounted on [Ca²⁺]i transient measurements using a Dvorak-Stotler chamber situated in a temperature-controlled stagte (37° C.) of a Zeiss I M 35 inverted microscope were performed as previously described (145-147).

Statistics. All results are expressed as means±SE. Kaplan-Meier survival curves were compared between group using log-rank tests. Two-way analysis of variance was used to analyze the calcium transient and contraction results. Commercial software package were used for all statistical analysis (JMP version 4.05; SAS Institute, Cary, N.C.) or SPSS for Windows (version 11.5). Categorical differences were analyzed using the Mann-Whitney test. In all analyses, P<0.05 was taken to be statistically significant.

### Example 1

Inducible and cardiac specific expression of A₁-AR. The inventors generated human A₁-AR transgenic (A₁-TG) mice controlled by an inducible cardiac-specific promoter with binding sites for the tetracyline transactivating factor (tTA). Gene expression was initiated by crossing six founder A₁-TG lines with mice that expressed tTA in the heart (MHC-tTA) (FIG. 1A). By immunoblotting, four of the five founder lines showed robust A₁-AR protein expression (FIG. 1B). Expression of A₁-AR was confirmed by radioligand binding in cardiac membranes using the A₁-AR ligand, DPCPX (FIG. 1C). Quantification of immunoblots and radio-ligand binding indicated that these A₁-AR transgenic lines expressed the A₁-AR at 500-1000 fold above endogenous A₁-AR level. Specificity was confirmed by competition with A₁-AR antigenic peptide and with excess non-radioactive A₁-AR binding ligand (FIG. 1C and data not shown).

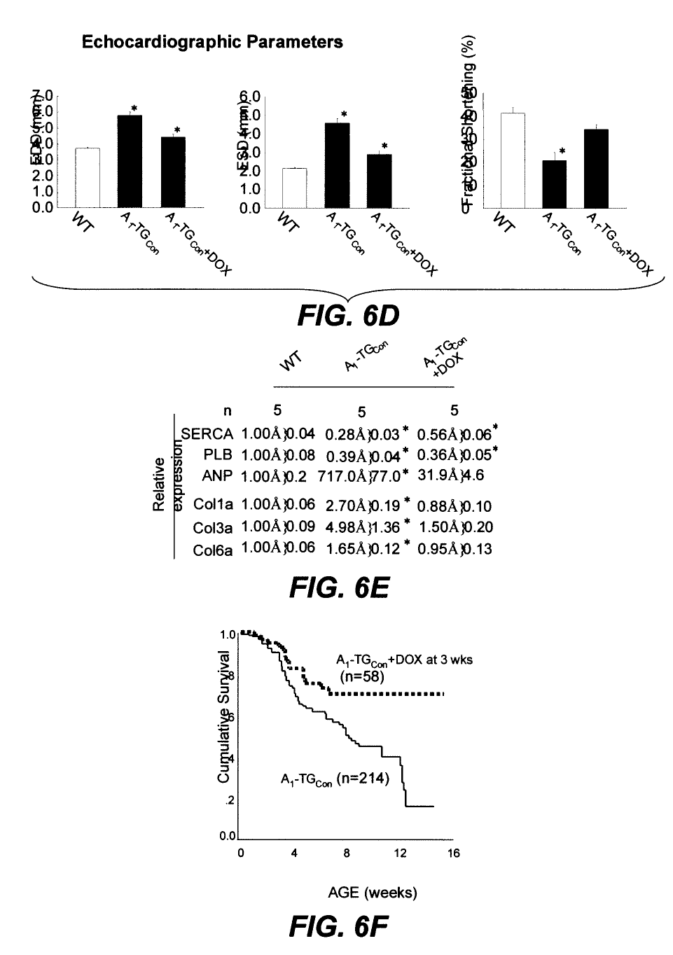

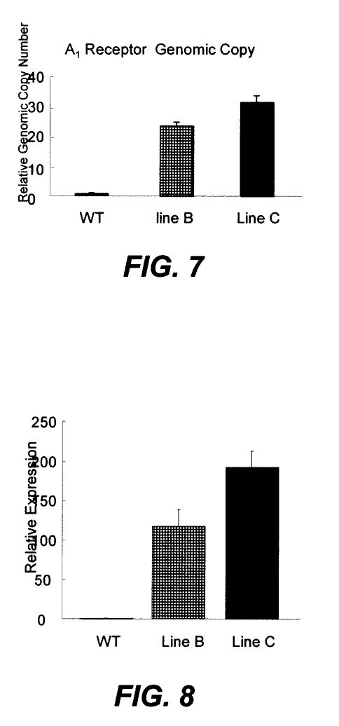

A₁-TG lines B and C were chosen for further characterization. Real-time PCR showed that both lines had similar genomic copy numbers and expressed transgene messages at similar levels (FIGS. 7 and 8). Human A₁-AR mRNA was not detected in other organs such as brain, lung, kidney and liver and gene expression of other AR subtypes (A₂ₐ, A_{2b}- and A₃-) were identical when compared with WT heart (data not shown).

### Example 2

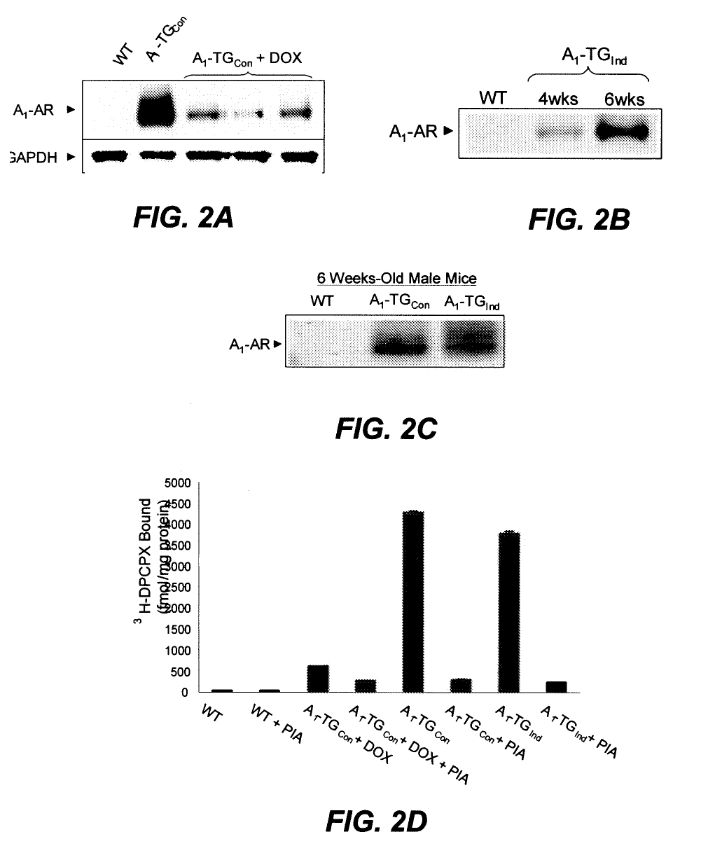

Doxycyline-regulated A₁-AR expression. The stable tetracycline analog, doxycycline (DOX), inhibits tTA transactivation. The inventors determined a minimal dose of DOX (300 mg/kg of mouse diets) that attenuated A₁-AR expression, which, in turn prevented cardiomyopathy. As shown in FIG. 2A, when DOX was continuously administered to pregnant mothers and subsequently to the offspring, cardiac A₁-AR expression was significantly attenuated at six-week of age comparing to constitutively expressed A₁-AR TG mice (A₁-TG_{Con}). To generate inducible A₁-AR transgenic mice (A₁-TG_{Ind}), DOX was removed when mice reached three-weeks of age. Time course studies showed that A₁-AR was fully re-expressed at six-weeks of age. At six weeks of age, A₁-AR protein expression in A₁-TG_{Ccon} and A₁-TG_{Ind} was identical (FIGS. 2B and C). DOX inhibition and A₁-AR induction are confirmed by A₁-AR binding assays (FIG. 2D).

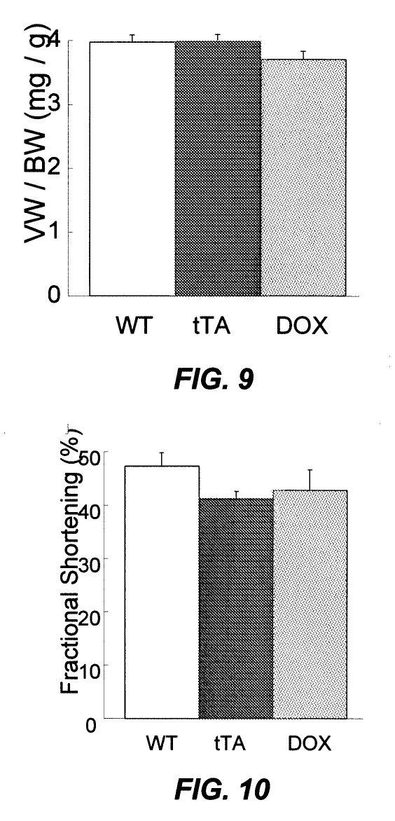

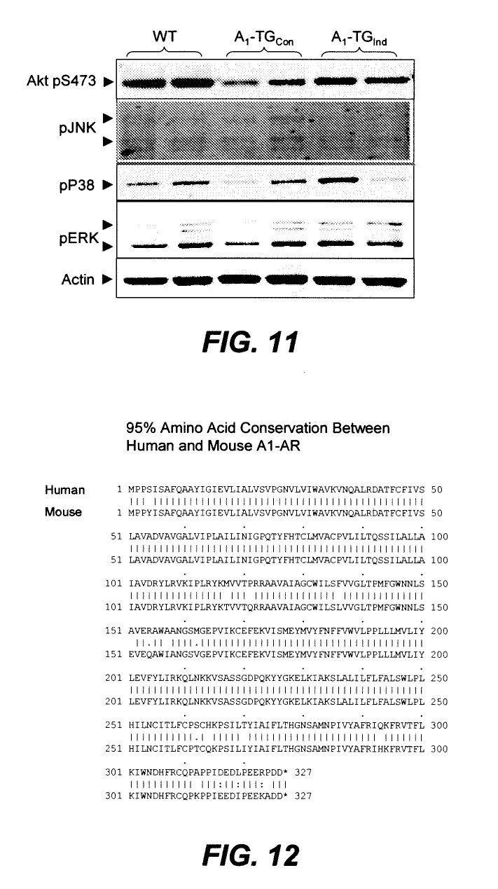

As reported previously,³³ the MHC-tTA mouse line expressed tTA at very low levels. The inventors discovered that tTA expression in WT mice did not affect mouse heart weight and function up to 12 weeks. Similarly, the amount of DOX used in the study did not affect WT mouse heart size or function (FIGS. 9 and 10).

### Example 3

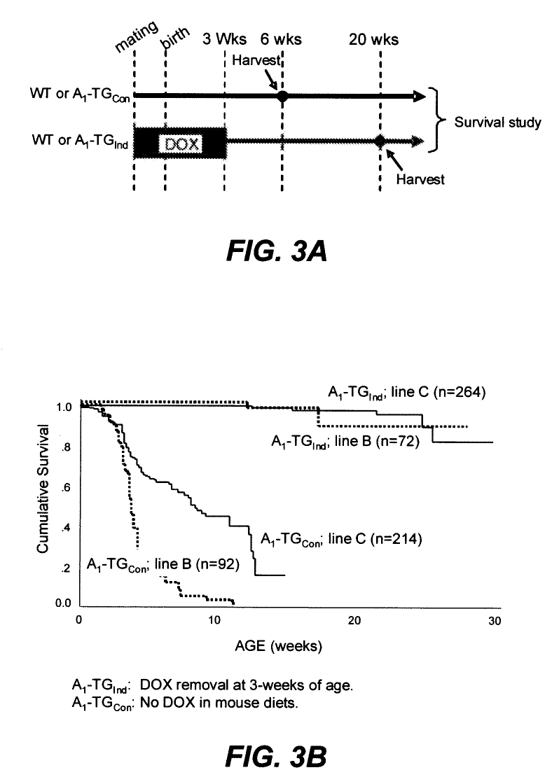

Constitutive and Induced Overexpression of A₁-AR. As shown by the induction scheme (FIG. 3A), A₁-AR was either expressed constitutively in the absence of DOX (A₁-TG_{Con}) or expression was induced (A₁-TG_{Ind}) by removing DOX at three weeks of age. Constitutive A₁-AR overexpression in two A₁-TG lines led to development of a dilated cardiomyopathy and high mortality in both male and female mice. Almost all A₁-TG_{Con} mice died within 6-12 weeks depending on the founder line (FIG. 3B). It should be noted that the higher mortality rate of line B was associated with a higher A₁-AR protein expression (FIG. 1B). Mice died of apparent congestive heart failure (post-mortem cardiac hypertrophy and dilation; pleural effusion). Both male and female mice showed similar mortality rate and phenotype (data not shown). In contrast, when A₁-AR expression was delayed until mice reached 3 weeks of age, over 90% of A₁-TG_{Ind} mice survived 30 weeks or longer despite comparable levels of A₁-AR overexpression. The inventors chose to use line C for the remaining physiological and biochemical studies because this line had the lowest transgene expression level and afforded longer survival.

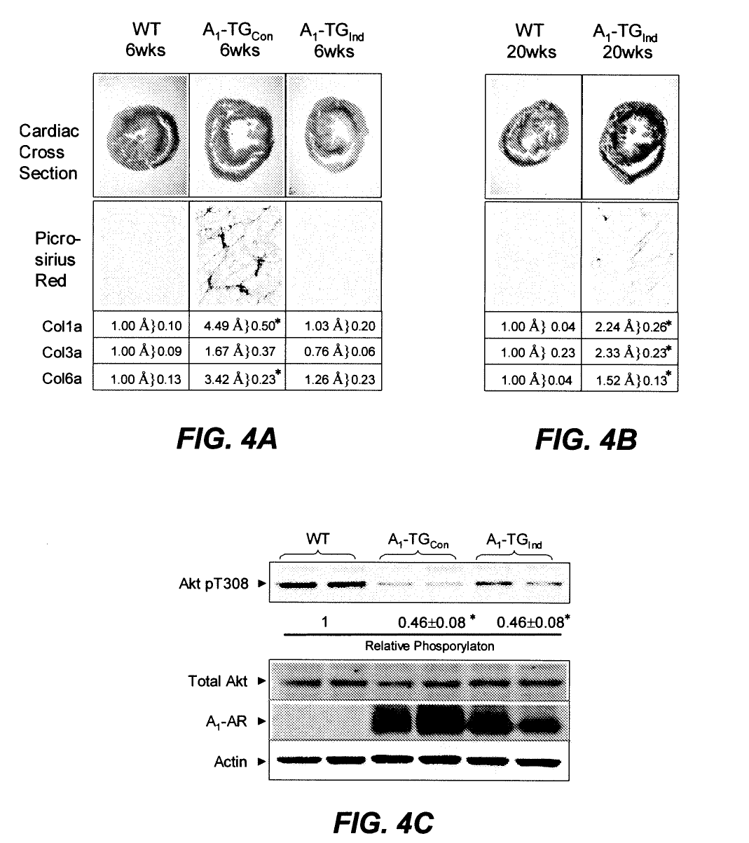

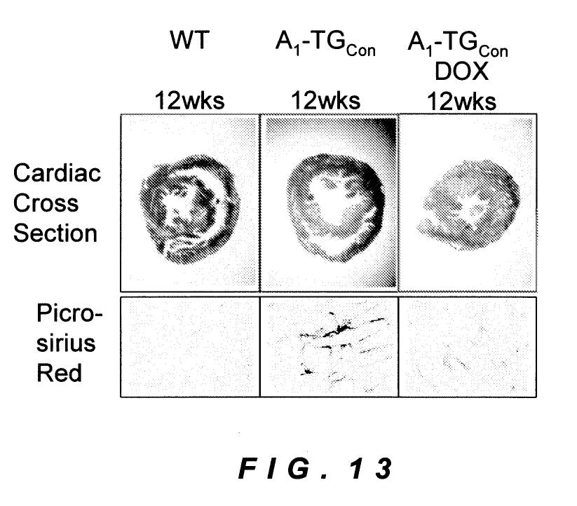

The inventors assessed cardiac functions and physical cardiac parameters when A₁-TG_{Con} or A₁-TG_{Ind} mice reached 6 weeks of age A₁-TG_{Con} developed early and profound cardiac dilatation, diminished ventricular function and marked bradycardia (FIG. 4A and Table 1). However, the inventors did not detect significant arrhythmia using surface ECG measurements (data not shown). In addition, the inventors determined the expression of genes frequently associated with cardiomyopathy. Data showed that expression of atrial natriuretic peptide (ANP) and collagen genes were enhanced in A₁-TG myocardium (FIG. 4A and Table 1). On the other hand, expression of the calcium handling genes, SERCA and phospholamban were decreased (Table 1). These mice also demonstrated extensive fibrosis by Picrosirus Red staining and enhanced expression of collagen genes (FIG. 4A). In contrast, when A₁-AR induction was delayed until 3 weeks of age, A₁-TG_{Ind} mice had a normal phenotype at six weeks of age as demonstrated by ventricular weight and cardiac function. There was, however, a small but statistically significant decrease in heart rate (Table 1). It should be noted that the marked reduction in heart rate was detected in both anesthetized resting mice and in conscious restrained mice. Delaying A₁-AR expression until mice reached 3-weeks of age, A₁-TG_{Ind} mice showed significantly improved heart rate and cardio-parameters when compared to A₁-TG_{Con} mice. To assess the effects of A₁-AR overexpression on Akt phosphorylation, the inventors probed heart extracts from 6 week-old male mice using antibodies that recognize phosphorylated Akt at Thr308. A₁-AR over-expression significantly decreased in basal Akt phosphorylation in both A₁-TG_{Con} and A₁-TG_{Ind} mice (FIG. 4C). Consistently, phosphorylation of Akt Ser473 was also decreased (FIG. 11). In contrast, phosphorylation of map kinases (Erk1/2, JNK1/2 and p38) were not altered (FIG. 11).

TABLE 1
Organ weights, Echocardiograohic Real-
time PCR data of 6 weeks old Mice.

| | | |
|---|---|---|
| WT | A₁-TG_{Con} | A₁-TG_{nd} |

Organ weights

| | | | |
|---|---|---|---|
| n | 8 | 10 | 7 |
| Body weight, g | 21.8 ± 0.8 | 17.8 ± 0.6* | 22.6 ± 0.8* |
| Ventricular/Body weight | 4.13 ± 0.09 | 7.45 ± 0.28* | 4.17 ± 0.18* |
| Lung/Body weight | 6.85 ± 0.22 | 10.51 ± 0.47* | 6.90 ± 0.21 |

Echocardiographic data

| | | | |
|---|---|---|---|
| n | 8 | 9 | 8 |
| Heart Rate, beats/min | 411 ± 11 | 108 ± 8* | 341 ± 13* |
| Heart Rate, beats/min | 580 ± 11 | 257 ± 7* | 345 ± 10* |
| (conscious) | | | |
| LVEDD, mm | 350 ± 0.07 | 5.56 ± 0.30* | 3.62 ± 0.07* |
| LVESD, mm | 1.90 ± 0.07 | 4.14 ± 0.37* | 2.20 ± 0.08* |
| Fractional shortening, % | 45.7 ± 2.1 | 26.3 ± 3.6* | 39.4 ± 1.5* |

Real-time PCR (relative expression)

| | | | |
|---|---|---|---|
| n | 5 | | 5 |
| SERCA | 1.00 ± 0.06 | 0.12 ± 0.01* | 0.59 ± 0.05* |
| PLB | 1.00 ± 0.08 | 0.23 ± 0.01* | 0.86 ± 0.05* |
| ANP | 1.00 ± 0.3 | 248 ± 52* | 7.9 ± 1.5* |

### Example 4

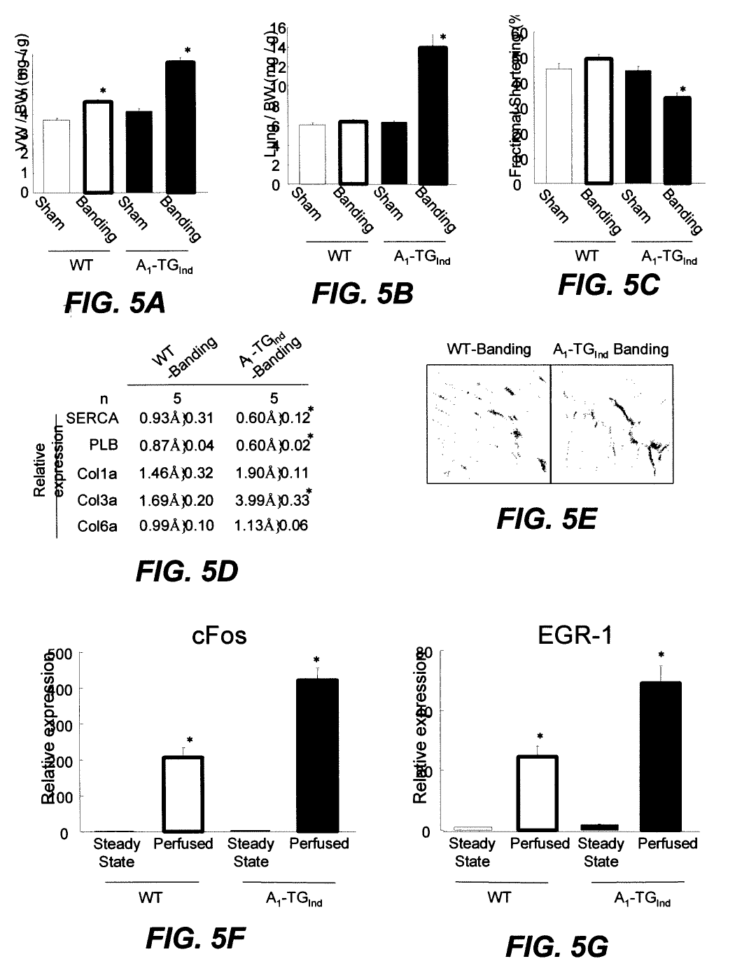

Effect of induced A₁-AR expression responding to pressure overload. Since A₁-TG_{Ind} mice had normal cardiac morphology and function at 6 weeks, the inventors assessed whether A₁-AR overexpression was cardioprotective in the presence of pressure overload induced by aortic banding. The inventors measured LV systolic pressure immediately before and after being banded and pressure gradient between wild-type and A₁-TG_{Ind} mice were similar (data not shown). As shown in FIGS. 5A, 5B and 5C, 4 weeks after surgery, aortic banding accelerated the hypertrophic response to pressure overload and further decreased fractional shortening when compared with non-transgenic controls. In addition, aortic banding markedly reduced the level of expression of the calcium handling genes SERCA and phospholamban (FIG. 5D), markedly enhanced fibrosis, and effected a significant decrease in heart rate in mice overexpressing the A₁-AR transgene (FIG. 5E).

Based on these results, the inventors next evaluated cardiac morphology and function in older A₁-TG_{Ind} mice. Consistent with the maladaptive effects of A₁-AR overexpression in young mice with aortic banding, at 20 weeks of age, A₁-TG_{Ind} mice developed ventricular enlargement, fibrosis, decreased fractional shortening and changes in the expression of calcium handling genes (FIG. 4B and Table 2). Importantly, at this age, a marked reduction in heart rate was detected in anesthetized resting mice when compared to heart rates of 6 week-old mice (Table 2). Interestingly, heart rates in conscious restrained mice did not differ between the two age groups.

TABLE 2
Organ Weight, Echocardiographic Real-
time PCR data of 20 weeks old Mice

| | |
|---|---|
| WT | A₁-TG_{ind} |

Organ weights

| | | |
|---|---|---|
| n | 5 | 10 |
| Body weight, g | 30.0 ± 1.4 | 28.6 ± 0.7 |
| Ventricular/Body weight | 3.94 ± 0.17 | 5.12 ± 0.19* |
| Lung/Body weight | 6.06 ± 0.17 | 6.45 ± 0.12 |

Echocardiographic data

| | | |
|---|---|---|
| n | 5 | 7 |
| Heart Rate, beats/min | 467 ± 31 | 144 ± 3* |
| Heart Rate, beats/min | 591 ± 4 | 333 ± 8* |
| (conscious) | | |
| LVEDD, mm | 364 ± 0.10 | 5.03 ± 0.13* |
| LVESD, mm | 1.81 ± 0.15 | 3.43 ± 0.10* |
| Fractional shortening, % | 50.4 ± 3.2 | 31.8 ± 1.1* |

Real-time PCR (relative expression)

| | | |
|---|---|---|
| n | 5 | 5 |
| SERCA | 1.00 ± 0.05 | 0.53 ± 0.02* |
| PLB | 1.00 ± 0.03 | 0.68 ± 0.05* |
| ANP | 1.00 ± 0.1 | 138.5 ± 31.9* |

Finally, to obviate the effects of heart rate on the changes in cardiac biology in the transgenic mice, the inventors next assessed the expression of early response genes, cFos and EGR-1, in Langendorff perfused A₁-TG_{Ind} hearts. Under equal paced conditions, hemodynamic parameters (left ventricular developed pressure, diastolic pressure, +dp/dt, and −dp/dt) were similar between wild-type control hearts and A₁-TG_{Ind} hearts (Table 3).

TABLE 3
Langendorff perfusion hemodynamics in A₁-TG_{Ind} and wild-type mouse
hearts (6 weeks-old male mice, N = 5).

| | | Coronary | Systolic | Diastolic | | | Developed |
|---|---|---|---|---|---|---|---|
| | Perfusion | Flow | Pressure | Pressure | | | Pressure |
| | Pressure | (ml/min) | (mmHg) | (mmHg) | + dp/dt | − dp/dt | (mmHg) |
| WT | 55 cm | 0.9 ± 0.3 | 56.1 ± 10.2 | 5.2 ± 2.8 | 2799 ± 675 | 1365 ± 302 | 50.9 ± 10.9 |
| A₁- | 55 cm | 1.3 ± 0.4 | 56.3 ± 4.2 | 7.2 ± 1.9 | 2854 ± 284 | 1250 ± 107 | 49.1 ± 4.8 |
| TG_{Ind} | | | | | | | |
| WT | 120 cm | 1.6 ± 0.3 | 85.6 ± 16.0 | 6.1 ± 3.0 | 3977 ± 954 | 2004 ± 439 | 79.4 ± 16.9 |
| A₁- | 120 cm | 2.3 ± 1.1 | 81.2 ± 6.2 | 15.1 ± 7.0 | 3501 ± 290 | 1595 ± 148 | 66.1 ± 5.3 |
| TG_{Ind} | | | | | | | |

LV-developed pressure was calculated by subtracting the LV diastolic pressure from the LV systolic pressure.
One way ANOVA analysis vs. WT showed no significance at P < 0.05 setting.

In preliminary studies, real-time quantitative PCR showed that serum rapidly induced cFos and EGR-1 expression in neonatal-myocyte derived cell line, H9C2, within 30 minutes (data not shown). As seen in FIGS. 5F and 5G, at steady-state, levels of cFos and EGR-1 in the mouse hearts were low and no differences were observed between wild-type and A₁-TG mice. However, the administration of a high perfusion pressure and therefore of increased cardiac load resulted in a rapid induction of cFos and EGR-1 expression. Importantly, the induction of cFos and EGR-1 expression was twice as high in A₁-TG mice when compared with wild-type controls.

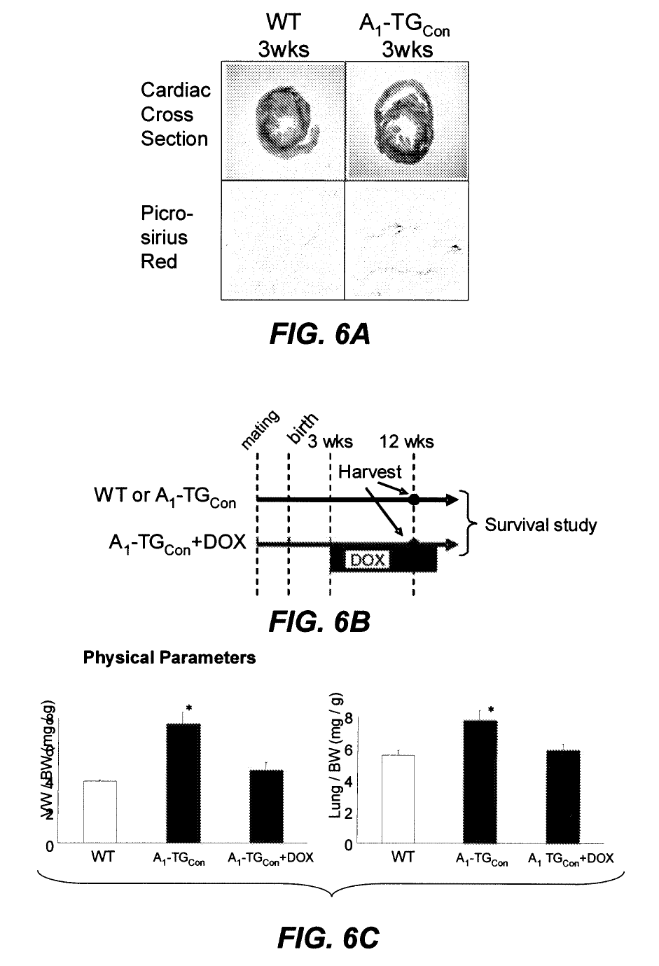

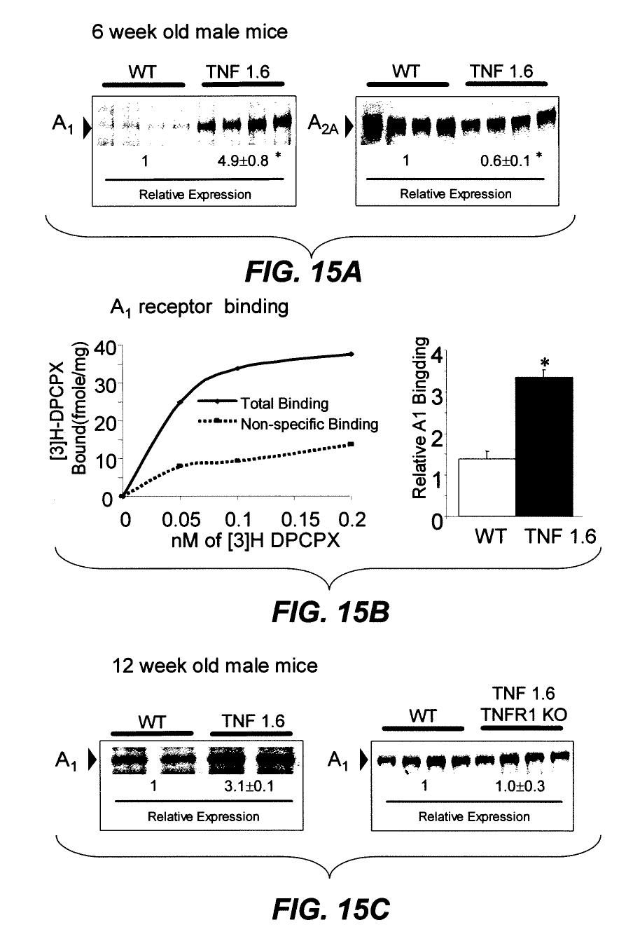

Effects of DOX treatment. By three weeks post-birth, A₁-TG_{Con} mice demonstrated enlargement of the left ventricular cavity and fibrosis (FIG. 6A). To determine whether the cardiomyopathy induced by A₁-AR overexpression was reversible, A₁-TG_{Con} mice were fed DOX beginning at 3 weeks in order to inhibit A₁-AR transgene expression (FIG. 6B). Assessment of cardiac function at 12 weeks demonstrated that attenuation of A₁-AR expression in the A₁-TG_{Con} mice normalized ventricular weight, increased fractional shortening and modulated LV dimension (FIGS. 6C and 6D). In addition, DOX reversed collagen staining, corrected gene expression profile towards normal levels and enhanced the survival of A₁-TG_{Con} mice (FIG. 6E, 6F and FIG. 13), indicating that the functional pathology in this model was largely reversible.

In order to investigate the adverse effects of exclusive activation of A₁-AR expression on cardiac morphology and function, the inventors created transgenic mice in which A₁-AR expression could be temporally regulated, by crossing mice harboring the α-MHC promoter driving very low levels of the tet trans-activator with mice harboring a transgene construct consisting of the human A₁-AR gene linked to an attenuated mouse α-MHC promoter that was inactive in the heart except when induced.³³ This novel construct provided the inventors the unique opportunity to evaluate the effects of A₁-AR activation overexpression beginning during prenatal development or after maturity as well as providing the ability to “turn-off” transgene expression to assess the reversibility of physiologic or morphologic changes.

Constitutive activation or overexpression (i.e. in the absence of DOX) of A₁-AR was demonstrated to result in a marked increase in the ventricular/body weight ratio, significant cardiac dilatation, diminished contractility, altered expression of cardiomyopathy-associated genes by six weeks of age and a significant mortality with all transgenic mice being dead by 15 weeks of age. In contrast, when expression of the A₁-AR transgene was inhibited until 3 weeks after birth, survival at 30 weeks was not adversely effected and cardiac function and morphology were normal at 6 weeks, although there was a modest but significant reduction in resting heart rate. However, 6 week-old A₁-AR mice (in which expression was activated at 3 weeks), were discovered to not able to tolerate pressure overload, as banding resulted in markedly enhanced hypertrophy, diminished cardiac function—changes that were not observed in banded wild-type non-transgenic controls. These physiological changes were discovered to be accompanied by diminished expression of calcium handling genes and enhanced expression of ANP and collagen genes.

The inventors discovery of profound adverse consequences of constitutive A₁-AR activation or overexpression could be partially reversed by “turning-off” the A₁-AR transgene with the administration of DOX. To address the concerns that the level of the A₁-AR overexpression in the present experiments might be “super-physiologic”, the inventors evaluated A₁-TG mice in which 80% of the transgene were suppressed using a sub-optimum DOX dose. The inventors demonstrated that mice with lower A₁-AR expression had a normal phenotype up to 20 weeks, but these mice remained unable to tolerate the stress of pressure overload (data not shown).

By contrast with constitutive overexpression of A₁-AR in C57/B16 mice, constitutive activation or overexpression of the A₃-AR in FVB mice also effected unfavorable changes in the cardiac phenotype that were consistent with the results in the previous experiment. Founders with high levels of A₃-AR died within 4 weeks of birth due to cardiac failure while heterozygote mice with moderate overexpression of the A₃-AR demonstrated cardiac hypertrophy, dilation, decreased function, recapitulation of the fetal gene program, and a significant decrease in heart rate at 14 to 17 weeks of age.^{30,44} By contrast, low levels of A₃-AR expression were not discovered to be associated with significant changes in morphology or function at young ages; although they did demonstrate a significant reduction in adenylyl cyclase activity30, first-degree AV block. 44 and altered sinus nodal and atrioventricular nodal function.²⁹

The inventors discovered that cardiac failure in the mice overexpressing the A₁-AR was associated with the development of hypertrophy, fibrosis, ANP induction and decreased SERCA and phospholamban expression. The inventors discovered a marked decrease in the expression of SERCA in the A₁-TG_{Ind} mice even before the onset of any changes in left ventricular size or function. In contrast to other heart failure models, the development of left ventricular hypertrophy and dysfunction was not associated with a change in MAP kinase activity, a key signaling protein in the development of cardiac hypertrophy (FIG. 11). In addition, the activity of Akt decreased (FIG. 4).

The development of cardiac hypertrophy and dilatation in FVB mice overexpressing the A₁-AR was discovered to be closely related to the A₁-AR-induced decrease in heart rate. Indeed, the profound bradycardia was detected in mice with both constitutive and inducible activation or overexpression of the A₁-AR and was demonstrated to contribute to the development of cardiomyopathy in these animals, demonstrating that selective activation or overexpression of the A₁-AR or A₃-AR receptor has salutary benefits on cardioprotection without adversely effecting cardiac morphology or function when heart rate does not change; however, the inventors discovered that cardiac dysfunction occurs when the heart rate is either moderately or markedly decreased. Assessment of the role of heart rate in effecting changes in cardiac function and morphology in the presence of A₁-AR overexpression is challenging because pacemakers small enough for a mouse are not commercially available.

The inventors assessed, if in the presence of increased load, A₁-AR TG hearts would demonstrate a rapid and robust increase in early response gene expression that was independent of heart rate. The inventors discovered that, in the presence of ventricular pacing, Langendorff perfused hearts from both A₁-AR TG and wild type mice demonstrated an increase in cFos and EGR-1 expression; however, the level of expression induced in the A₁-AR TG was twice that seen in the wild-type controls (FIGS. 5F and 5G). This demonstrates that intrinsic molecular changes occur in response to an increase in pressure load in the presence of A₁-AR activation or overexpression that are independent of heart rate are associated with alterations in the fetal gene program that is a characteristic feature of the heart failure phenotype. Further, the inventors have discovered that heart-rate related changes in cardiac function and morphology can contribute to the cardiomyopathy observed in the animals, in particular the profound cardiac dysfunction observed in older animals.

The inventor's discoveries have important implications on the therapeutic use of selective adenosine receptor agonists (i.e. agonists which activate only one adenosine receptor subtype at a time) in subjects with cardiovascular disease. For example, when used chronically, doses of selective adenosine agonists that do not decrease heart rate should be chosen for clinical investigation and individual patients should be observed carefully to insure that variations in genetic background do not result in unique effects on heart rate in selected patient populations. In addition, the inventors have demonstrated that use of therapeutic agents which have a more balanced effect, i.e. activate both the A₁- and A₂-ARs are likely to be safer in patients with normal or compromised cardiac function.

### Example 5

In Examples 1 to 4, the inventors demonstrated the effect of temporal changes in expression of A₁- and A_{2A}R on heart related changes and cardiac function and demonstrated that disproportionate modification of one or the other of A₁- or A_{2A}R contributes to cardiomyopathy. As such, the inventors discovered that adenosine therapy which activates only one adenosine receptor subtype is not a beneficial therapeutic strategy for adenosine therapy, but rather simultaneous activation of at least two adenosine receptor subtypes, for example both A₁- and A_{2A}-ARs simultaneously is of benefit to subjects with normal or compromised cardiac function.

Far less, however, is known about the role of adenosine in the failing heart, for example on myocardial infarction. Adenosine levels have been reported to be elevated in patients with heart failure.⁷² In addition, subjects with heart failure who harbor a nonsense mutation in the AMP deaminase gene, resulting in high levels of muscle adenosine, have a markedly improved survival when compared with patients having the wild-type genotype.⁷³ By contrast, recent studies have reported that high levels of over expression of the A₁- or A₃-AR in the heart can have untoward effects.⁷⁴⁻⁷⁶ Indeed, it was discovered that over expression of high levels of the A₃-AR results in the development of a dilated cardiomyopathy. However, information is not available regarding changes in adenosine system in the failing heart. In this Example, the inventors evaluated the myocardial adenosine system in a well-studied mouse model of heart failure, the TNF 1.6 mouse⁷⁷⁻⁷⁹, which demonstrate LV dilation, marked diminution in heart rate and fractional shortening, and significant increases in LV end-diastolic pressure and ventricular weight.

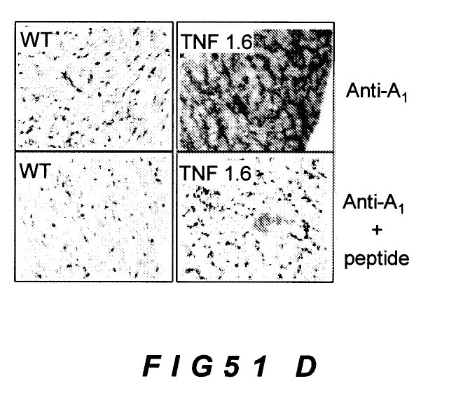

Adenosine levels in TNF 1.6 mice. Baseline echocardiographic and hemodynamic data for TNF 1.6 mice are found in Table 4, which demonstrate that in 6-weeks old TNF 1.6 mice, changes in cardiac morphology and function which are associated with a substantial decrease in myocardial adenosine levels in TNF 1.6 mice as compared with gender-matched non-transgenic controls (FIG. 14A). Average of 70% decrease were also seen in young TNF 1.6 mice (3 weeks of age) prior to the onset of profound morphologic and hemodynamic changes and in older 22 week old mice with end-stage disease (FIG. 14B). The inventors compared the traditional Wollenberger clamp method^{83,84} to a modified rapid pinch-excision method, which preserving adenosine for subsequent measurement (data not shown).

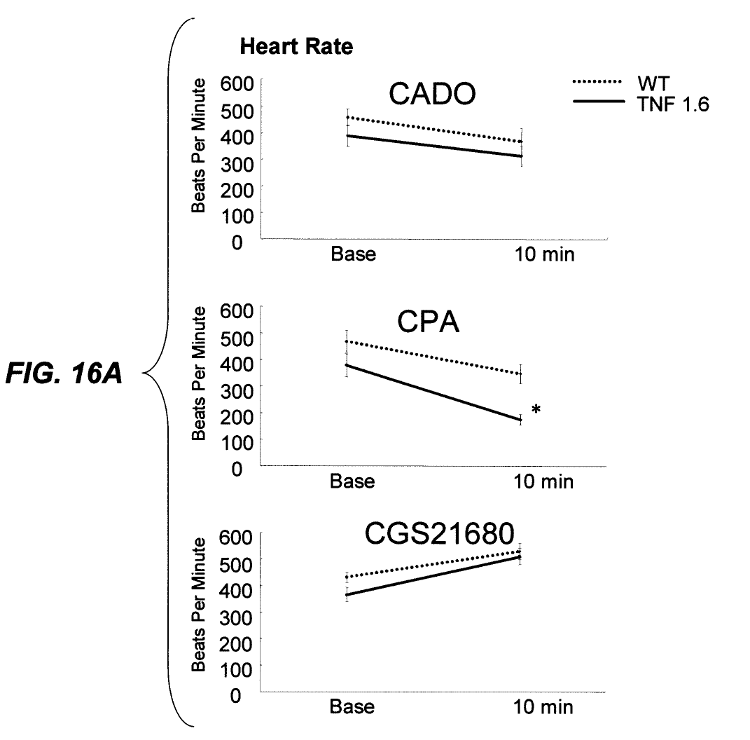

Regulation of A₁ and A_{2A} ARs. The change in myocardial adenosine levels was associated with sub-type selective alterations in the expression of ARs. As seen in FIG. 15A, the inventors detected A₁ AR expression was enhanced 4.9 fold in TNF 1.6 myocardium. By contrast, A_{2A} AR was decreased by 40% in the same samples (FIG. 15A). The changes in protein levels for A₁ AR was largely independent of steady state levels of the A₁ AR mRNA (WT vs. TNF 1.6: 100%+/−23% vs. 70%+/−32%, n=5). Consistent with analysis of receptor levels by Western blotting, A₁-AR binding was significantly higher in the TNF 1.6 myocardium than in age- and gender-matched controls (FIG. 15B). It is not unexpected that the Western blotting would give higher values than radioligand binding because Western blotting assesses the total amount of protein in a tissue whereas radioligand binding only detects receptors that are in the correct conformation and are present on the membrane surface. Thus, receptors that are “down-regulated” would not be identified by radioligand binding assays. More importantly, the inventors have discovered, using two independent methods an increase in the amount of A₁-AR. Finally, when A₁ AR levels were measured in 12 week old male TNF 1.6 mice that had been crossed with TNFR1 knockout mice, A₁ AR levels were determined not to be changed as compared with age-matched WT controls (FIG. 15C), demonstrating ablation of TNFR1, but not TNFR2, blocks cardiotoxic effects in TNF 1.6 mice.⁸²

TABLE 4
Echochardiographic, hemodynamic and organ weights of mice. Organ
weights (N = 4), echocardiography (N = 4) and hemodynamic
data (N = 15) in 6-weeks-old wild-type and TNF 1.6 male mice.

| | | |
|---|---|---|
| Wild type | TNF 1.6 | p value |

Echocardiographic data

| | | | |
|---|---|---|---|
| n | 4 | 4 | |
| LVEDD, mm | 3.53 ± 0.14 | 4.68 ± 0.14 | <0.05 |
| LVESD, mm | 2.03 ± 0.04 | 3.69 ± 0.13 | <0.05 |
| Fractional shortening, % | 42.2 ± 2.6 | 21.1 ± 1.0 | <0.05 |

Hemodynamic data

| | | | |
|---|---|---|---|
| n | 15 | 15 | |
| Heart Rate, beats/min | 453 ± 10 | 380 ± 10 | <0.001 |
| Mean AoP, mmHg | 62.3 ± 0.8 | 57.8 ± 0.7 | <0.001 |
| LVEDP, mmHg | 1.6 ± 0.2 | 7.5 ± 0.6 | <0.001 |
| dP/dtₘₐₓ, mmllgisec | 8380 ± 201 | 5162 ± 273 | <0.001 |
| dP/dtₘᵢₙ, mmHg/sec | 6425 ± 180 | 4616 ± 154 | <0.001 |

Organ weights

| | | | |
|---|---|---|---|
| n | 4 | 4 | |
| Body weight, g | 22.0 ± 0.9 | 25.5 ± 1.0 | <0.05 |
| Ventricular weight, mg | 90.1 ± 6.8 | 156.4 ± 8.5 | <0.01 |
| Lung weight, mg | 145.8 ± 11.7 | 176.7 ± 7.8 | <0.01 |

Values were mean ± SEM and P value was analyzed with non-parametric method.

To identify the cell types that expressed A₁ AR protein, the inventors stained wild-type and TNF 1.6 myocardium with an anti-A₁-AR antibody. As shown in FIG. 15D, in wild-type and TNF 1.6 mouse hearts, A₁-AR was expressed throughout the myocardium, but was more abundant in the TNF 1.6 hearts. Importantly, all cell types including cardiac myocytes had enhanced A₁-AR staining in TNF 1.6 hearts. The specificity for binding was shown by the fact that binding could be inhibited by competition with a selective peptide (FIG. 15D).

A₁ receptor-specific functional response. To determine whether the changes in A₁-AR levels had functional significance in TNF 1.6 mice, the inventors determined the chronotropic response to the selective A₁-AR agonist, 2-chloro-N⁶-cyclopentanyladenosine (CPA). In wild-type mice, CPA effectively decreased heart rate. However, as seen in FIG. 16A, the inventors discovered that CPA produced a far more robust decrease in heart rate in TNF 1.6 mice as compared with age- and gender-matched wild-type controls. The inventors discovered that CPA was only slightly increased arterial pressure and cardiac contractility (FIG. 16B), whereas infusion of the non-selective adenosine agonist, 2-chloroadenosine (CADO) or the A_{2A} receptor selective agonist, 2-p-(2-carboxyethyl)phenethylamino-5′-N-ethylcaroxamino adenosine hydrochloride (CGS21680) had a similar effects in TNF 1.6 and wild-type mice.

Myocardial adenosine levels in models of left ventricular dysfunction. In order to insure that the changes in adenosine levels in mice with heart failure secondary to TNFα over expression were reflective of changes in LV function and not simply a phenomenon associated constitutive over expression of TNFα, the inventors evaluated adenosine levels in other models of heart failure and maladaptive cardiac remodeling: mice with LV dysfunction secondary to over expression of CSQ and mice with cardiac dysfunction secondary to surgically-induced chronic pressure overload. As shown in FIG. 17A, mice over-expressing CSQ and mice with surgically induced cardiac pressure overload both demonstrated significant decreases in myocardial adenosine levels as compared with the appropriate wild-type or sham-operated (for aortic constriction model) controls. In addition, the inventors discovered an inverse linear relationship between LV performance, as measured by fractional shortening, and adenosine levels across the three heart failure models (FIG. 17C). In contrast to TNF 1.6 mice, the inventors discovered cardiac TNF\acute{α} expression in both CSQ and banded mice was almost undetectable despite significant decreases in left ventricular function (FIG. 17D).

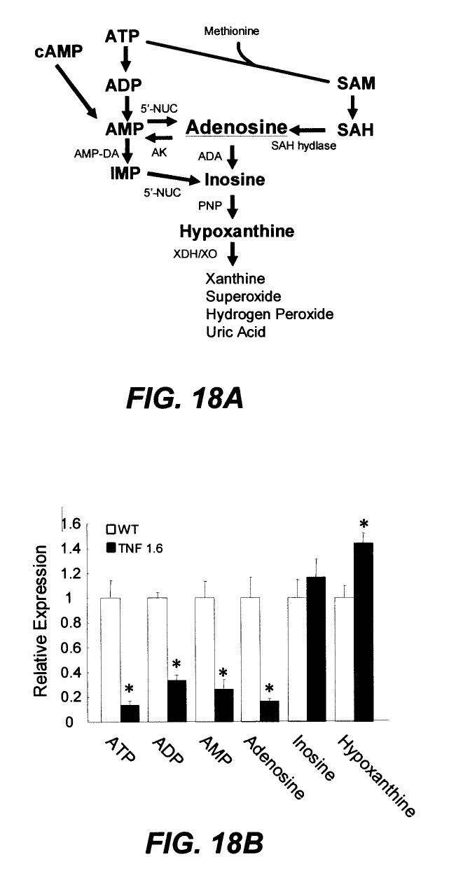

Levels of adenosine precursors in TNF 1.6 mice. Although controversial, adenosine production in disease is thought to occur through the metabolism of ATP (FIG. 18A)⁸⁵. The inventors discovered that mice over-expressing TNFα had a profound decrease in myocardial levels of ATP, ADP and AMP as compared with wild-type controls (FIG. 18B). By contrast, the inventors determined that there was no change in the level of inosine, but a significant increase in the levels of hypoxanthine, both catabolic products of adenosine metabolism.

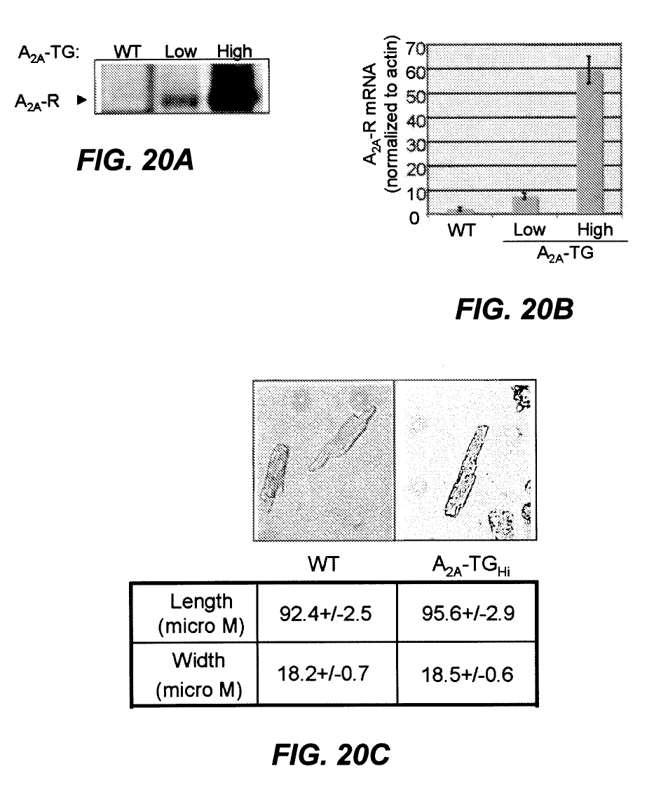

Expression profiling of TNF 1.6 mice. In order to identify changes in enzymes that might contribute to the production of either adenosine, or adenosine precursors, the inventors performed gene profiling using an Affymatrix platform. Of the 5962 genes that were screened in mRNA isolated from the hearts of 6 week old TNF 1.6 and wild-type control mice, the inventors identified and discovered two ATP synthase components whose expression were significantly decreased in the TNF 1.6 mice: ATP synthase, H+ transporting, mitochondrial FO complex, subunit F (Atp5j), ATP synthase, H⁺ transporting, mitochondrial F1 complex, subunit O (Atp5o) (data not shown). The inventors confirmed these results by real-time PCR quantification (FIG. 20A).

In contrast, the inventors detected that the mRNA levels of ectonucleotide pyrophosphatase/phosphodiesterase 2 (Enpp2) were substantially increased in the myocardium. Enpp2, also known as autotoxin, is an integral membrane enzyme that degrades extracellular ATP, ADP, AMP and cAMP to adenosine. Real-time PCR on mRNA isolated from the same gender and age matched mice confirmed the findings from the affymatrix displays (FIG. 20B).

Finally, the inventors determined the expression of the two major enzymes involved in adenosine catabolism, purine nucleoside phosphoyrlase (PNP) or xanthine dehydrogenase/xanthine oxidase (XDH/XO). Real-time PCR data showed that both enzymes were significantly enhanced in TNF 1.6 myocardium when compared with wild-type controls (FIG. 20C). To the best of our knowledge, this is the first evidence of PNP up regulation in the failing heart, although XO has been shown to be up-regulated in cases of TNF\acute{α} overexpression. Up regulation of ENPP2 and XO proteins in TNF 1.6 myocardium was confirmed by immunoblotting with specific anti-ENPP2 and anti-XO antibodies (FIG. 20D).

### Example 6

Creation of transgenic mice overexpressing the A_{2A}-Adenosine Receptor. The inventors created mice overexpressing the A_{2A}-Adenosine receptor by placing the human A_{2A}-adenosine receptor (A_{2A}-AR) cDNA under the control of a cardiac-specific promoter as described previously (127). Using an anti-A_{2A}-R antibody, the inventors analyzed levels of A_{2A}-R expression in both wild-type and 15 lines of transgenic mice. Based on these measurements, the transgenic lines were classified as low expression (2-5×, A_{2A}-TG_{Lo}) or high expression (more than 50×, A_{2A}-TG_{Hi}) (FIG. 10A). RT-PCR showed that A_{2A}-R mRNA was also increased in these strains (FIG. 20B).

A_{2A}-R overexpression increased cardiac contractility. As seen in Table 5, the inventors determined that overexpression of the A_{2A}-AR resulted in a small but significant increase in left ventricular mass (ventricular wt/body wt) at 8 to 12 wks in mice with high levels of A_{2A}-AR overexpression but not in those with low levels of A_{2A}-AR overexpression. However, measurements of single cell morphology showed no increase in either ventricular myocyte width or length (FIG. 20C). Echocardiography demonstrated an increase in heart rate and fractional shortening as well as a significant decrease in left ventricular end-systolic dimension (LVESD) in mice with both high and low levels of A_{2A}-AR overexpression (Table 5).

TABLE 5
Basal Physiology of High and Low Expression of A_{2A}-transgenic mice.
Organ weights and echocardiography data in mice expressing high levels (A₂G-TG_{Hi}) or low
levels (A₂G-TG_{Lo}) of A_{2A}-R. Mice were examined at 8-12 weeks.

| | | | P value | | P value |
|---|---|---|---|---|---|
| | WT | A_{2A-}TG_{Hi} | vs Wt | A_{2A-}TG_{L0} | vs Wt |
| n | 15 | 17 | | 12 | |
| Ventricular/Body weight | 3.86 +/− 0.04 | 4.57 +/− 0.2 | <0.001 | 4.04 +/− 0.09 | NS |
| Heart Rate | 372 +/− 13 | 457 +/− 13 | <0.001 | 494 +/− 11 | <0.001 |
| LVEDD, mm | 3.28 +/− 0.06 | 3.04 +/− 0.01 | NS | 2.94 ± 0.008 | <0.005 |
| LVESD, mm | 1.86 +/− 0.09 | 1.35 +/− 0.12 | <0.005 | 1.33 +/− 0.09 | <0.005 |
| Fractional shortening, % | 43.6 +/− 1.9 | 56.6 +/− 2.5 | <0.005 | 55.05 +/− 2.4 | <0.005 |

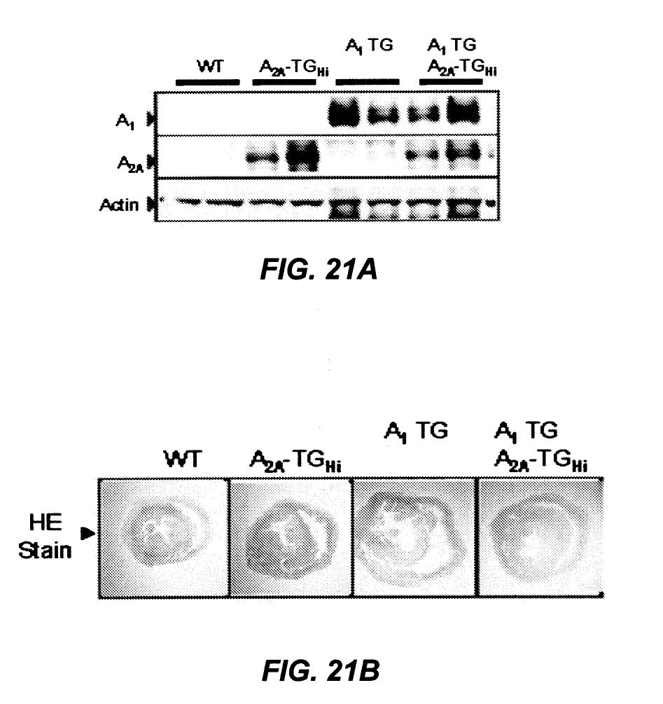

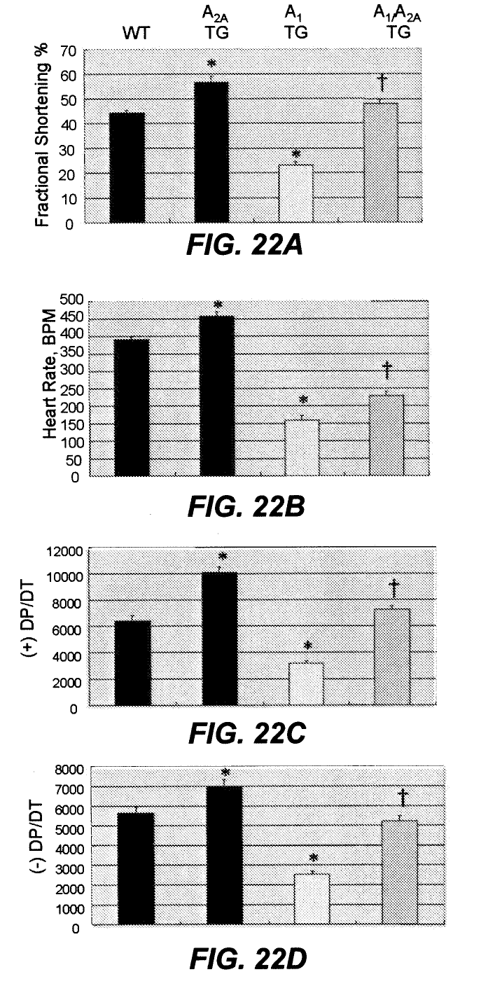

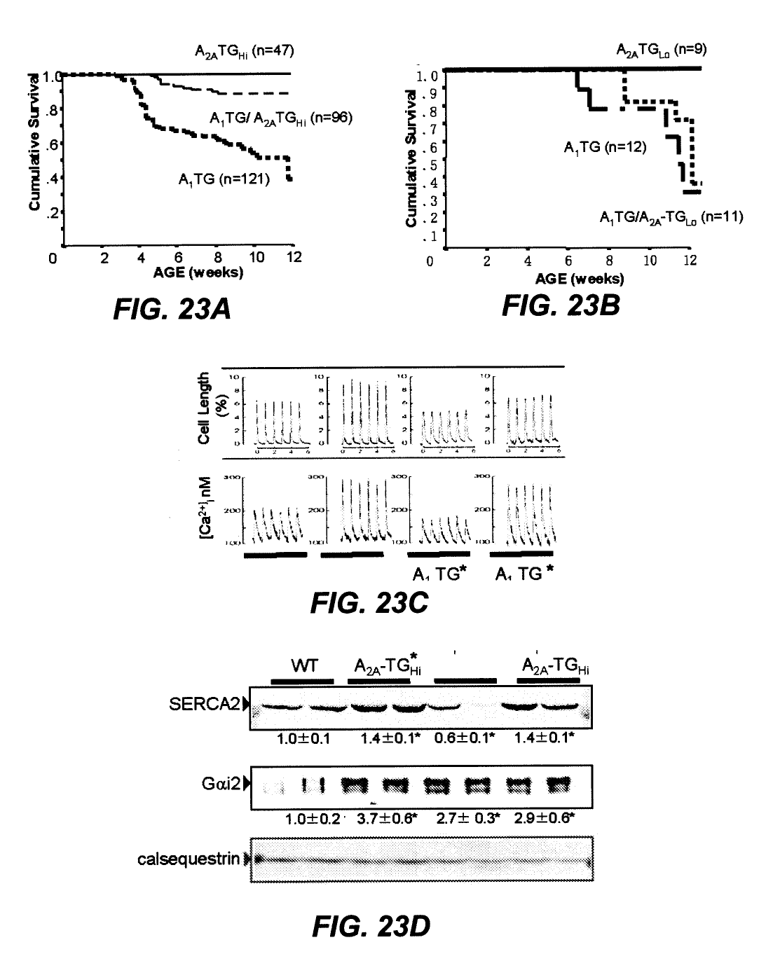

Overexpression of the A_{2A}-AR prevents the development of the cardiomyopathic phenotype in mice overexpressing the A₁-AR. In a previous study, the inventors demonstrated that constitutive overexpression of the A₁-AR decreased cardiac contractility in FVB mice (127). The discovery that A_{2A}-AR increased cardiac contractility led the inventors to analyse if the adverse effects of A₁-AR overexpression was due at least in part to a lack of balance in the endogenous stoichiometry of the A₁- and A_{2A}-ARs. In order to test this hypothesis, the inventors generated created double recombinants harboring both the A₁-AR and A_{2A}-R_{Hi} transgenes (A₁/A_{2A}-TG_{Hi}). As seen in FIG. 21A, co-expression of the A₁-AR and A_{2A}-AR transgenes did not influence the expression of either the A₁-AR or A_{2A}-AR receptors when compared with mice in which either the A₁-AR- or A_{2A}-AR transgenes were expressed alone. Also, immunofluorescence microscopy of isolated myocytes confirmed that both receptors co-localized to the sarcolemmal surface (data not shown). While mice overexpressing A₁-AR alone developed profound cardiac dilatation, the inventors surprisingly discovered that co-expression of A_{2A}-AR prevented the profound cardiac dilatation in A₁-TG mice (FIG. 21B). Furthermore, as seen in FIG. 22, co-expression of the A₁-AR and A_{2A}-AR transgenes significantly improved cardiac hemodynamics when compared with mice overexpressing the A₁-AR. Indeed, fractional shortening (FS), left ventricular end-diastolic pressures, +dp/dt, and −dp/dt were similar in A₁/A_{2A}-TG mice and wild-type controls. However, heart rate remained depressed in the A₁/A_{2A}-TG mice, albeit at a rate significantly higher than that seen in the A₁-TG mice. Furthermore, as seen in FIG. 23A, co-expression of the A₁-AR and A_{2A}-AR_{Hi} transgenes significantly improved survival comparing to mice overexpressing A₁-AR alone. By contrast, co-expression of the A₁-R with the A_{2A}-AR TG_{Lo} had no effect on the cardiac phenotype when compared with A₁-TG mice (FIG. 23B and data not shown).

Overexpression of A_{2A}-R enhances myocyte [Ca²⁺]i transport. In view of the marked increase in contractility in the A_{2A}-TGHi mice and their ability to prevent heart failure in the A₁-TG mice, the inventors next assessed if mice harboring the A_{2A} transgene have enhanced Ca²⁺ handling. In order to test this the inventors first compared Ca²⁺ handling in myocytes isolated from A_{2A}-TG_{Hi}, A₁-TG, and wild-type non-transgenic littermates. As seen in FIG. 23C and Table 6, when compared to WT myocytes, constitutive overexpression of A_{2A}-AR resulted in significant higher systolic (p<0.0001) but not diastolic [Ca²⁺i (p<0.60). Maximal contraction amplitudes (p<0.0001), maximal shortening (p<0.0003) and relengthening velocities (p<0.006) were all higher in myoctyes overexpressing A_{2A}-R (Table 6). Sarcoplasmic reticulum (SR) Ca²⁺ uptake activity was significantly faster (p<0.0001).

By contrast, myocytes in which A₁-AR was constitutively overexpressed had significantly lower systolic (p<0.0005) and diastolic [Ca²⁺]i (p<0.0007) at all [Ca²⁺]o examined (Table 6). Altered [Ca²⁺]i transients in myocytes with constitutively overexpressed A₁-AR resulted in decreased maximal contraction amplitudes as well as maximal shortening and relengthening velocities (p<0.0001 for all 3 parameters; Table 6). Furthermore, the t1/2 of [Ca²⁺]i transient decline, an estimate of sarcoplasmic reticulum Ca²⁺ uptake (135), was significantly prolonged in A₁-AR TG myocytes when compared with WT (p<0.0001) myocytes.

Co-overexpression of A₁- and A_{2A}-R ameliorates the abnormal [Ca²⁺]i handling seen in the A₁-R TG mycoytes. When analyzing myocyte function in the dual A₁-/A_{2A}-AR transgenic mice, the inventors surprisingly discovered that the overexpression of the A_{2A}-R significantly ameliorated the contractile abnormalities observed in A₁-AR TG myocytes (p<0.0001 for maximal contraction amplitude, maximal shortening and relengthening velocities, Table 6). In addition, maximal contraction amplitude of A₁/A_{2A}-AR TG myocytes was significantly higher (p<0.015) than that observed in WT myocytes but lower (p<0.05) than that measured in A_{2A}-R overexpressed myocytes (Table 6).

TABLE 6
Effects of Adenosine receptor overexpression on [Ca²⁺]_{I} transients, myocyte
concentration and calcium handling protein expression. For calcium transient and
contraction measurements, numbers in parentheses are myocytes pooled from 4-5
mice/genotype group.

| | | | |
|---|---|---|---|
| | | | A₁-TG |
| WT | A_{2A}-TG_{Hi} | A₁-TG | A_{2A}-TG_{Hi} |

Calcium Transients [Ca²⁺]₀ mM
Systolic [Ca^{2±}]_{I}, nM

| | | | | |
|---|---|---|---|---|
| 0.6 | 167 ± 10 (20) | 205 ± 13* (28) | 110 ± 9* (20) | 181 ± 14*^{$} (12) |
| 1.8 | 215 ± 10 (39) | 295 ± 14* (32) | 172 ± 12* (38) | 271 ± 22*^{$} (32) |
| 5.0 | 336 ± 11 (41) | 488 ± 18* (35) | 304 ± 30* (15) | 480 ± 80*^{$} (19) |

Diastolic[Ca²⁺]_{I}, nM

| | | | | |
|---|---|---|---|---|
| 0.6 | 118 ± 7 | 108 ± 6⁺ | 73 ± 9* | 94 ± 13* |
| 1.8 | 111 ± 7 | 116 ± 6⁺ | 95 ± 7* | 86 ± 11* |
| 5.0 | 131 ± 5 | 125 ± 5⁺ | 113 ± 11* | 85 ± 17* |

T_{r/2}[Ca2+]_{I} transient decline, ms

| | | | | |
|---|---|---|---|---|
| 0.6 | 216 ± 13 | 175 ± 11*⁺ | 307 ± 32* | 231 ± 22*^{$} |
| 1.8 | 174 ± 7 | 124 ± 7*⁺ | 243 ± 13* | 143 ± 8*^{$} |
| 5.0 | 151 ± 5 | 100 ± 4*⁺ | 200 ± 8* | 133 ± 8*^{$} |

Myocyte Contraction [Ca²⁺]⁰
Maximal contraction amplitude, 0/0 cell length

| | | | | |
|---|---|---|---|---|
| 1.8 | 6.80 ± 0.43 (56) | 9.07 ± 0.42*⁺(38) | 4.99 ± 0.45* (36) | 7.85 ± 0.41*^{$} (36) |
| 5.0 | 10.59 ± 0.44 (38) | 12.79 ± 0.57*⁺(49) | 7.17 ± 0.82* (11) | 11.92 ± 0.35*^{$} (22) |

Maximal shortening velocity, cell lengths

| | | | | |
|---|---|---|---|---|
| 1.8 | 1.05 ± 0.07 | 1.39 ± 0.06*⁺ | 0.63 ± 0.06* | 1.00 ± 0.05^{$} |
| 5.0 | 1.58 ± 0.10 | 1.84 ± 0.09*⁺ | 0.90 ± 0.13* | 1.53 ± 0.07^{$} |

Maximal relengthening velocity, cell length/s

| | | | | |
|---|---|---|---|---|
| 1.8 | 0.92 ± 0.07 | 1.17 ± 0.07*⁺ | 0.42 ± 0.04* | 0.75 ± 0.05^{$} |
| 5.0 | 1.29 ± 0.10 | 1.49 ± 0.09*⁺ | 0.68 ± 0.13 | 1.17 ± 0.05^{$} |

Selected Calcium Handling Protein Expression

| | | | | |
|---|---|---|---|---|
| SERCA2 | 1.00 ± 0.07 (7) | 1.42 ± 0.05*^{$} (4) | 0.63 ± 0.11* (7) | 1.37 ± 0.14*^{$} (4) |
| Na⁺ | 1.00 ± 0.03 (4) | 0.78 ± 0.09^{$} (4) | 0.43 ± 0.11* (4) | 0.71 ± 0.04*^{$} (4) |
| Pump | | | | |
| NCXI | 1.00 ± 0.12 (4) | 1.45 ± 0.15^{$} (4) | 0.83 ± 0.19 (4) | 1.32 ± 0.17^{$} (4) |

Mouse groups: wild-type (WT), A_{2A}-TG_{Hi}, A₁-TG) and A₁/A_{2A}-TG.
Values are means ± SE.
Two-way analysis of variance was used for analysis.
*p < 0.05, compared to WT;
^{$}p < 0.0001, A₁-TG vs. A₁-TG/A_{2A}-TG_{Hi};
⁺p < 0.05, A_{2A}-TG vs. A₁-TG/A_{2A}-TG_{Hi}.

Protein expression were determined by immunoblotting. Numbers in parentheses are number of hearts from each mouse group. Signal band intensities on each blot were normalized to the average intensity of that protein measured in wild-type hearts. One-way analysis of variance followed by Dunnett's test was used to analyze the results. *P<0.05, compared to WT; A₁-TG vs. A_{2A}-TG_{Hi} or A₁-TG vs. A₁/A_{2A}-TG_{Hp}

With respect to [Ca²⁺]i homeostasis, SR Ca²⁺ uptake activity (p<0.0001) and systolic (p<0.0001) but not diastolic [Ca²⁺]i (p<0.36) was improved by co-overexpression of A₁ and A_{2A}-AR when compared with constitutive overexpression of A₁-AR alone (Table 6). Diastolic [Ca²⁺]i was significantly lower in A₁/A_{2A} myocytes when compared to WT (p<0.0001) or A_{2A}-R overexpressed (p<0.0001) myocytes. The t1/2 of [Ca²⁺]i transient decline was significantly shorter in A₁/A_{2A} myocytes when compared to WT myocytes (p<0.009) but longer when compared to A_{2A}-AR overexpressed myocytes (p<0.002).

Effects of enhanced A_{2A}-AR signaling on biochemical pathways in the heart. In order to understand the mechanisms responsible for the enhanced contractility effected by increased A_{2A}-AR signaling, the inventors assessed the levels of proteins involved in Ca²⁺ homeostasis and G protein-coupled receptor signaling in the heart. As seen in FIG. 23D, the amount of SERCA₂ was significantly increased in hearts from A_{2A}-AR TGHi mice when compared with wild-type controls. The level of SERCA₂ protein was also significantly elevated in A₁/A_{2A}-TGHi mice but by contrast was significantly lower than WT controls in mice overexpressing the A₁-AR. The levels of the Na+ pump and NCX1 in the A_{2A}-TG mice had changing trends, but they were not statistically different from the WT mice. By contrast, mice overexpressing the A₁-AR had decreased Na+ pump protein levels. Surprisingly, the inventors discovered that co-overexpression of A_{2A}-AR in A₁-TG mice enhanced the expression of all three proteins involved in Ca²⁺ homeostasis when compared to mice expressing only A₁-AR (FIG. 23D and Table 6). However, the change in contractility seen in the A_{2A}-R TG_{Hi} mice, as well as in the A_{2A}-AR TG_{Lo} mice, was not associated with an increase in steady-state adenylyl cyclase activity (Data not shown). By contrast, levels of the G-inhibitory protein, Gαi-2, as well as the levels of mRNA encoding Gαi-2 were increased in all three experimental models (A_{2A}-TG_{Hi}, A₁-TG, A₁/A_{2A}-TG; FIG. 23D and data not shown).

Long term effects of enhanced A_{2A}-AR signaling. Although overexpression of the A_{2A}-AR markedly enhanced cardiac contractility in mice up to 12 weeks of age, the inventors demonstrated that long-term overexpression was not associated with an increase in cardiac contractility. Indeed, cardiac function was identical in 20-week-old transgenic and non-transgenic controls as seen by measurement of fractional shortening (42±2.0% WT, n=7 vs. 40.3±3.4% A_{2A}-TG_{Hi}, n=13, p=NS). By contrast with young mice overexpressing A_{2A}-AR, SERCA₂ levels were not elevated in 20 week old A_{2A}-TG_{Hi} mice (1.1±0.1, n=7) when compared with wild type controls (1.0±0.1, n=13, p=NS). Similarly, while phosphorylation (ie. activation) of Akt was significantly enhanced in young mice (12 weeks old) overexpressing the A_{2A}-AR transgene (1.03±0.2 WT, n=3 vs. 1.8±0.16, A_{2A}-TG_{Hi}, n=6, p<0.01), pAkt level were not elevated in 20 week old A_{2A}-R TG_{Hi} mice (0.91±0.12, n=9) when compared with wild type controls (1.12±0.15, n=7, p=NS). However, levels of Gαi remained elevated in the hearts of the 20-week-old A_{2A}-TG_{Hi} mice (1.9±0.3 A_{2A}-R TG_{Hi}, n=13 versus 1.0±0.1 WT, n=7, p<0.01).

In Examples 5 and 6, the inventors have demonstrated for the first time, using cardiac-restricted overexpression of the A_{2A}-AR in mice, the effects of A_{2A}-AR-signaling on cardiac morphology and function. The inventors have also demonstrated important limitations imposed by experiments utilizing receptor sub-type “selective” agonists and antagonists, i.e. studies which activate only one adenosine receptors subtype at a time.

The inventors have discovered that constitutive overexpression of the A_{2A}-AR in young mice resulted in super normal contractility which was associated with a modest increase in heart rate and a small but significant increase in LV mass. In myocytes isolated from hearts overexpressing the A_{2A}-AR, systolic but not diastolic [Ca²⁺]i was elevated and t1/2 of [Ca²⁺]i transient decline was much shorter when compared with wild-type controls and myocyte size did not change. These discoveries demonstrate enhanced SR Ca²⁺ uptake, resulting in increased SR Ca²⁺ content, more Ca²⁺ available for release per beat, and larger systolic [Ca²⁺]i values and twitch amplitudes in A_{2A}-AR overexpressed myocytes. SERCA₂ but not Na+-K+-ATPase or NCX1 expression was demonstrated to be increased in A_{2A}-AR over-expressed myocytes. Thus, the inventors have demonstrated that one major alteration induced by A_{2A}-AR overexpression was increased SERCA₂ expression and SR Ca²⁺ uptake activity.

The inventors discoveries are disparate from studies evaluating the role of adenosine receptor sub-type specific agonists and antagonists; which suggested that A_{2A}-AR-mediated inotropy was accompanied by only a small increase in Ca²⁺ transients. Furthermore, these earlier studies also suggested that A_{2A}-AR activation could increase shortening and the rate of maximal shortening in isolated myocytes without an effect on maximal rate of relaxation(137) while in isolated hearts, A_{2A}-AR activation could increase left ventricular pressure and the maximal rate of pressure development (+dP/dt/max) without influencing cardiac relaxation (132). In stark contrast, the inventors herein have demonstrated that enhanced contractility as well as enhanced relaxation—these effects being associated with increased expression of SERCA₂ and robust changes in Ca²⁺ handling. Importantly, the inventors have discovered that the changes in cardiac function after A_{2A}-AR overexpression were not due to an increase in heart rate, as demonstrated by the detection of enhanced contractility both in vivo as well as in isolated and paced myocytes.

Due to the inventors discovery of contrasting effects of A₁- and A_{2A}-AR overexpression on cardiac function, the inventors next assessed whether abnormalities in calcium homeostasis was also be responsible for the adverse effects of A₁-AR overexpression. The inventors demonstrated that the left ventricular myocyte contractility was severely depressed in A₁-TG myocytes. In addition, systolic [Ca²⁺]i in A₁-AR myocytes was lower at all 3 [Ca²⁺]o examined, demonstrating that SR Ca²⁺ uptake was diminished (138). The decreased SERCA₂ expression and prolonged t1/2 of [Ca²⁺]i transient decline in A₁-TG myocytes demonstrate defective SR Ca₂+ uptake activity, resulting in decreased SR Ca₂+ content and diminished twitch amplitudes. Diastolic [Ca²⁺]i was also discovered to be lower with A₁-AR overexpression, demonstrating accelerated Ca²⁺ efflux further contributes to reduced SR Ca²⁺ content and twitch amplitude.

As the inventors discovered that one of the major alterations in the excitation-contraction coupling in A_{2A}-TG myocytes was enhanced SERCA₂ expression and SR Ca²⁺ uptake, the inventors next assessed if enhanced A_{2A}-AR signaling ameliorated the negative inotropic effects of enhanced A₁-AR signaling. The inventors surprisingly discovered that co-overexpression or co-activation of A_{2A}-AR with overexpression or activation of A₁-AR improved cardiac contractility, decreased end-diastolic pressure, enhanced SERCA₂ expression and markedly improved survival as compared with mice overexpressing only the A₁-AR. The inventors also demonstrate these salutary benefits at the single cell level, demonstrating that co-expression of the A₁-AR and A_{2A}-AR ameliorated the marked cellular abnormalities found in the A₁-TG mice. As systolic [Ca²⁺]i and t1/2 of [Ca²⁺]i transient decline improved without a change in diastolic [Ca²⁺]i in the A₁-/A_{2A}-TG mice when compared with A₁-TG mice, the defects in SR Ca²⁺ uptake but not Ca²⁺ efflux pathways induced by constitutive A₁-AR overexpression were discovered to be influenced by co-overexpression of A_{2A}-AR. The inventors also discovered the ability of A_{2A}-AR signaling to ameliorate the adverse effects of A₁-AR overexpression in a dose-dependent manner, as crossing the A₁-TG with A_{2A}-AR TG_{Lo} mice had no effect on cardiac hemodynamics or outcomes despite an increase in contractility in the A_{2A}-TG_{Lo} mice.

Despite the salutary benefits of short-term overexpression of the A_{2A}-AR, the inventors discovered that long term benefits were not obvious as by 20 weeks of age, as contractility decreases substantially to levels less similar to those seen in WT controls.

The inventors also discovered that early activation of A_{2A}-AR signaling did not cause left ventricular dysfunction or pathology, as demonstrated by significant increase in Akt levels in young mice. Phosphorylation (activation) of Akt is known to have cardioprotective effects (141). The inventors demonstrated that while phosphorylation of Akt was enhanced in young mice overexpressing the A_{2A}-AR, it was not increased in older A_{2A}-TG mice with “normalization” of left ventricular function.

The inventors have demonstrated that the therapeutic usefulness of A_{2A}-AR overexpression can be predicated on “dose” as despite an inability to reverse left ventricular dysfunction caused by A₁-AR overexpression, low levels of A_{2A}-AR overexpression significantly increased cardiac contractility without causing an increase in either ventricular or atrial hypertrophy.

Taken together, the inventors have discovered that despite the fact that the A₁-AR and A_{2A}-AR signaling pathways affect different downstream events, the physiologic integrity of the heart, at least in a chronic sense, requires an ongoing balance between the activation of these two pathways. Indeed, the family of adenosine receptor subtypes is unusual in that it is the only group of G-protein coupled-seven trans-membrane spanning receptors in which a single ligand can bind to multiple receptor sub-types in the same tissue and in so doing mediate opposing signaling pathways—i.e. adenylyl cyclase activation in the case of the A_{2A}-AR and adenylyl cyclase inhibition via the A₁-AR. Thus, it would appear axiomatic that the chronic use of sub-type selective adenosine receptor agonists or antagonists might have unexpected consequences. The inventors discoveries should be considered when designing adenosine agonists and/or antagonists for chronic use in subjects with any cardiac dysfunction or cardiovascular disease.

### REFERENCES

All the references cited herein and throughout the application, including the specification are incorporated herein in their entirety by reference.
1. Peart J, et al., Am J Physiol Heart Circ Physiol. February 2003; 284(2):H519-527.
2. Zhao et al., Circulation. August 1993; 88(2):709-719.
3. Babbitt et al., Circulation. November 1989; 80(5):1388-1399.
4. Peart et al., Am J Physiol Heart Circ Physiol. November 2000; 279(5):H2166-2175.
5. Ross et al., J Am Coll Cardiol. Jun. 7, 2005; 45(11):1775-1780.
6. Wagner et al., Circulation. Feb. 17, 1998; 97(6):521-524.
7. Dubey et al., Circulation. Oct. 21, 1997; 96(8):2656-2666.
8. Stowe et al., J Cardiovasc Pharmacol. June 1997; 29(6):747-754.
9. Rongen et al., Hypertension. April 1996; 27(4):933-938.
10. Taddei et al., Am J Hypertens. January 1992; 5(1):29-31.
11. Liao et al., Circ Res. Oct. 17, 2003; 93(8):759-766.
12. Gastmann et al., Am J Cardiol. May 15, 2004; 93(10):1260-1264.
13. Yazaki et al., J Card Fail. August 2004; 10(4):316-320.
14. Loh et al., Circulation. Mar. 23, 1999; 99(11):1422-1425.
15. Kitakaze et al., Cardiovasc Drugs Ther. July 1998; 12(3):307-309.
16. Headrick et al., Am J Physiol Heart Circ Physiol. November 2003; 285(5):H1797-1818.
17. Sullivan et al., Br J Pharmacol. March 2001; 132(5):1017-1026.
18. Dobson et al., Cardiovasc Res. May 1997; 34(2):337-347.
19. Headrick et al., J Physiol. Jun. 15, 2003; 549(Pt 3):823-833.
20. Safran et al., Mol Cell Biochem. January 2001; 217(1-2):143-152.
21. Vinten-Johansen et al., Ann Thorac Surg. November 1999; 68(5):1942-1948.
22. Zhao et al., Am J Physiol. October 1997; 273(4 Pt 2):H1677-1687.
23. Todd et al., Ann Thorac Surg. November 1996; 62(5):1364-1372.
24. Reichelt et al., Circ Res. Feb. 18, 2005; 96(3):363-367.
25. Yang et al., Am J Physiol Heart Circ Physiol. March 2002; 282(3):H949-955.
26. Matherne et al., Proc Natl Acad Sci USA. Jun. 10, 1997; 94(12):6541-6546.
27. Guo et al., J Mol Cell Cardiol. April 2001; 33(4):825-830.
28. Gauthier et al., J Mol Cell Cardiol. January 1998; 30(1):187-193.
29. Kirchhof et al., Am J Physiol Heart Circ Physiol. July 2003; 285(1):H145-153.
30. Black et al., Circ Res. Jul. 26, 2002; 91(2):165-172.
31. Zhao et al., Dev Dyn. June 2001; 221(2):194-200.
32. Ng et al., Circ Res. June 1991; 68(6):1742-1750.
33. Sanbe et al., Circ Res. Apr. 4, 2003; 92(6):609-616.
34. Funakoshi et al., Circ Res. May 17, 2002; 90(9):959-965.
35. Funakoshi et al., Am J Physiol Heart Circ Physiol. June 2002; 282(6):H2159-2166.
36. MacDonnell et al., Circulation. Jun. 28, 2005; 111(25):3420-3428.
37. Piuhola et al., Am J Physiol Heart Circ Physiol. July 2003; 285(1):H112-118.
38. Eklund et al., Proc Natl Acad Sci USA. Jan. 30, 2001; 98(3):1194-1199.
39. Lopes et al., Br J Pharmacol. March 2004; 141(6):1006-1014.
40. Bristow et al., Mol Pharmacol. March 1989; 35(3):295-303.
41. Higuchi et al., Am J Physiol Heart Circ Physiol. Oct. 14, 2005.
42. Higuchi et al., Circulation. Apr. 20, 2004; 109(15):1892-1897.
43. Shusterman et al., Am J Physiol Heart Circ Physiol. June 2002; 282(6):H2076-2083.
44. Fabritz et al., Cardiovasc Res. Jun. 1, 2004; 62(3):500-508.
45. Redfern et al., Proc Natl Acad Sci USA. Apr. 25, 2000; 97(9):4826-4831.
46. Redfern et al., Nat Biotechnol. February 1999; 17(2):165-169.
47. Klinger et al., Cell Signal. February 2002; 14(2):99-108.
48. Krieg et al., Am J Physiol Heart Circ Physiol. December 2002; 283(6):H2322-2330.
49. Qin et al., Am J Physiol Heart Circ Physiol. February 2003; 284(2):H727-734.
50. Regan et al., Am J Physiol Heart Circ Physiol. March 2003; 284(3):H859-866.
51. Hautala et al., Pflugers Arch. January 2002; 443(3):362-369.
52. Magga et al., Life Sci. 1998; 63(12):1005-1015.
53. Cornelius et al., Hypertension. December 1997; 30(6):1348-1355.
54. Kovacic-Milivojevic et al. Circ Res. April 1997; 80(4):580-588.
55. Sadoshima J et al., Annu Rev Physiol. 1997; 59:551-571.
56. von Harsdorf R, et al., J Clin Invest. Sep. 1, 1997; 100(5):1294-1304.
57. Nakagawa et al., J Clin Invest. September 1995; 96(3):1280-1287.
58. Semeniuk et al., February 2003; 284(2):H425-430.
59. Dorn et al., Proc Natl Acad Sci USA. May 25, 1999; 96(11):6400-6405.
60. Sah R, et al., Circulation. Apr. 16, 2002; 105(15):1850-1856.
61. Freeman et al., Am J Physiol Heart Circ Physiol. January 2001; 280(1):H151-159
62. Harding et al., Proc Natl Acad Sci USA. May 8, 2001; 98(10):5809-5814.
63. Rockman et al., Proc Natl Acad Sci USA. Jun. 9, 1998; 95(12):7000-7005.
64. Sommerschild et al., Acta Anaesthesiol Scand. October 2000; 44(9):1038-1055.
65. Headrick J P, et al., Am J Physiol Heart Circ Physiol. November 2003; 285(5):H1797-1818.
66. Lasley R D, et al., J Mol Cell Cardiol. January 1990; 22(1):39-47.
67. Zhao et al., Circulation. August 1993; 88(2):709-719.
68. Zhao et al., Cardiovasc Res. February 1994; 28(2):270-279.
69. Peart J, et al., Am J Physiol Heart Circ Physiol. November 2000; 279(5):H2166-2175.
70. Reichelt M E, et al., Circ Res. Feb. 18, 2005; 96(3):363-367.
71. Matherne G P, et al., Proc Natl Acad Sci USA. Jun. 10, 1997; 94(12):6541-6546.
72. Funaya H, et al., Circulation. Mar. 18, 1997; 95(6):1363-1365.
73. Loh E, et al., Circulation. Mar. 23, 1999; 99(11):1422-1425.
74. Gauthier N S et al., J Mol Cell Cardiol. January 1998; 30(1):187-193.
75. Black et al., Circ Res. Jul. 26, 2002; 91(2):165-172.
76. Yang Z, et al., Am J Physiol Heart Circ Physiol. March 2002; 282(3):H949-955.
77. Funakoshi et al., Circ Res. May 17, 2002; 90(9):959-965.
78. Funakoshi et al., Am J Physiol Heart Circ Physiol. June 2002; 282(6):H2159-2166.
79. Kubota et al., Circ Res. October 1997; 81(4):627-635.
80. Harding V B, et al., Proc Natl Acad Sci USA. May 8, 2001; 98(10):5809-5814.
81. Jones et al., J Clin Invest. Apr. 1, 1998; 101(7):1385-1393.
82. Higuchi Y, et al., Circulation. Apr. 20, 2004; 109(15):1892-1897.
83. Leunissen R L, et al., J Appl Physiol. December 1968; 25(6):769-771.
84. Wollenberger A, et al., Pflugers Arch Gesamte Physiol Menschen Tiere. 1960; 270:399-412.
85. Jackson E K, et al., Annu Rev Physiol. 2004; 66:571-599.
86. Hettinger et al., J Neurochem. July 1998; 71(1):221-230.
87. Nie Z, et al., Mol Pharmacol. September 1997; 52(3):456-464.
88. Ramkumar V, et al., Mol Pharmacol. November 1991; 40(5):639-647.
89. Hettinger-Smith B D, et al., J Neurochem. November 1996; 67(5):1921-1930.
90. Abbracchio M P, et al., Eur J Pharmacol. Nov. 2, 1992; 227(3):317-324.
91. Zou A P, et al., Hypertension. January 1999; 33(1 Pt 2):511-516.
92. Ashton et al., Cardiovasc Res. March 2003; 57(3):715-726.
93. Headrick J P, et al., J Physiol. Jun. 15, 2003; 549(Pt 3):823-833.
94. Zimpfer M, et al., J Clin Invest. February 1981; 67(2):430-438.
95. Broughton A, et al., Cardiovasc Res. May 1980; 14(5):245-253.
96. Liao et al., Circ Res. Oct. 17, 2003; 93(8):759-766.
97. Shusterman V, et al., Am J Physiol Heart Circ Physiol. June 2002; 282(6):H2076-2083.
98. Varani K, et al., Faseb J. February 2003; 17(2):280-282.
99. Laghi-Pasini F, et al., J Intern Med. December 2003; 254(6):591-596.
100. Headrick J, et al., Pflugers Arch. October 1988; 412(6):618-623.
101. Headrick J P. et al., J Mol Cell Cardiol. June 1996; 28(6):1227-1240.
102. Kitakaze M, et al., Cardiovasc Res. January 1993; 27(1):18-27.
103. Budde J M, et al., Cardiovasc Res. August 2000; 47(2):294-305.
104. Todd J, et al., Ann Thorac Surg. November 1996; 62(5):1364-1372.
105. Vinten-Johansen J, et al., Basic Res Cardiol. July 2005; 100(4):295-310.
106. Meyer et al., Hypertension. March 2001; 37(3):862-868.
107. Zucchi R, et al., Cardiovasc Res. Feb. 1, 2002; 53(2):326-333.
108. Boucher J, et al., Diabetologia. March 2005; 48(3):569-577.
109. Bollen M, et al., Crit Rev Biochem Mol Biol. 2000; 35(6):393-432.
110. Rockman H A, et al., Nature. Jan. 10, 2002; 415(6868):206-212.
111. Kitakaze M, et al., Expert Opin Investig Drugs. November 2000; 9(11):2519-2535.
112. Kitakaze M, et al., Jpn Circ J. April 1999; 63(4):231-243.
113. Minhas K M, et al., Circ Res. Feb. 3, 2006; 98(2):271-279.
114. Amado L C, et al., J Mol Cell Cardiol. September 2005; 39(3):531-536.
115. Berry C E, et al., J Physiol. Mar. 16, 2004; 555(Pt 3):589-606.
116. Cappola T P, et al., Circulation. Nov. 13, 2001; 104(20):2407-2411.
117. Machida Y, et al., Am J Physiol Heart Circ Physiol. February 2003; 284(2):H449-455.
118. Chen Y, et al., Circ Res. Oct. 17, 2003; 93(8):691-693.
119. Yang, D., et al., (2006) J Clin Invest 116, 1913-23.
120. Klinger, et al., (2002) Cell Signal 14, 99-108.
121. Norton, G. R., et al., (1999) Am J Physiol 276, H341-9.
122. Cross, et al., (2002) Am J Physiol Heart Circ Physiol 283, H1562-8.
123. Yang, Z., et al., (2002) Am J Physiol Heart Circ Physiol 282, H949-55.
124. Morrison, R. R. et al., (2000) Am J Physiol Heart Circ Physiol 279, H1071-8.
125. Reichelt, M. E., et al., (2005) Circ Res 96, 363-7.
126. Lankford, A. R., et al., (2006) Am J Physiol Heart Circ Physiol 290, H1469-73.
127. Funakoshi, H., et al., (2006) Circulation 114, 2240-50.
128. Gauthier, N. S., et al., (1998) J Mol Cell Cardiol 30, 187-93.
129. Matherne, G. P., et al., (1997) Proc Natl Acad Sci U.S.A 94, 6541-6.
130. Kilpatrick, E. L., et al., (2002) Am J Physiol Heart Circ Physiol 282, H1035-40.
131. Lasley, R. D., et al., (2001) Am J Physiol Heart Circ Physiol 280, H1660-6.
132. Monahan, T. S., et al., (2000) Am J Physiol Heart Circ Physiol 279, H1472-81.
133. Shryock, J., et al., (1993) Circ Res 72, 194-205.
134. Headrick, J. P., et al., (2003) Am J Physiol Heart Circ Physiol 285, H1797-818.
135. Zhang, X. Q., et al., (1999) J Appl Physiol 87, 2143-50.
136. Woodiwiss, A. J., et al., (1999) Am J Physiol 276, H1434-41.
137. Dobson, J. G., et al., (1997) Cardiovasc Res. 34, 337-47.
138. Ahlers, B. A., et al., (2005) J Appl Physiol 98, 2169-76.
139. Foerster, K., et al., (2003) Proc Natl Acad Sci U.S.A 100, 14475-80.
140. Gaudin, C., et al., (1995) J Clin Invest 95, 1676-83.
141. Heineke, J. & Molkentin, J. D. (2006) Nat Rev Mol Cell Biol. 7, 589-600.
142. Sanbe, A., et al., (2003) Circ Res. 92, 609-16.
143. Funakoshi, et al., (2002) Circ. Res. 90, 959-65.
144. Funakoshi, H., et al., (2002) Am J Physiol Heart Circ Physio. 282, H2159-66.
145. Tucker, A. L., et al., (2006) Am J Physiol Heart Circ Physiol 291, H2199-209.
146. Song, J., et al., (2005) Am J Physiol Heart Circ Physiol 288, H2342-54.
147. Song, J., et al., (2002) Am J Physiol Heart Circ Physiol 283, H576-83.
148. Funakoshi H, et al., Circ Res. May 17, 2002; 90(9):959-965.
149. Funakoshi H, et al., Am J Physiol Heart Circ Physiol. June 2002; 282(6):H2159-2166.
150. Tang Z, et al., J Mol Cell Cardiol. April 2004; 36(4):515-530.
151. Lopes L V, et al., Br J Pharmacol. March 2004; 141(6):1006-1014.
152. Bristow M R, et al., Mol Pharmacol. March 1989; 35(3):295-303.
153. Higuchi Y, et al., Am J Physiol Heart Circ Physiol. Oct. 14, 2005.
154. Higuchi Y, et al., Circulation. Apr. 20, 2004; 109(15):1892-1897.
155. Leunissen R L, et al., J Appl Physiol. December 1968; 25(6):769-771.
156. Wollenberger A, et al., Pflugers Arch Gesamte Physiol Menschen Tiere. 1960; 270:399-412.
157. Jackson E K, et al., J Pharmacol Exp. Ther. Mar. 9, 2006.

1-3. (canceled)

4. A method for enhancing cardiac function in a subject comprising;
(a) selecting a subject in need of, or currently being treated an adenosine agonist therapy;
(b) administering to the subject a pharmaceutical composition comprising at least one agent which co-activates both an A₁-adenosine receptor (A₁-AR) and an A_{2A}-adenosine receptor (A_{2A}-AR), or a combination of at least one agent which activates an A₁-adenosine receptor (A₁-AR) and at least one agent which activates an A_{2A}-adenosine receptor (A_{2A}-AR), wherein the level of activation of A₁-AR is about the same as the level of activation of A_{2A}-AR.

5. The method of claim 4, wherein the subject is first diagnosed as having, or at risk of having a cardiac dysfunction, wherein a subject identified as having, or at risk of having a cardiac dysfunction is then treated for cardiac dysfunction according to the method of claim 4.

6. The method of claim 4, wherein the pharmaceutical composition is free of a sodium-hydrogen exchanger inhibitory compound.

7. The method of claim 4, wherein the subject in need is selected from the following subjects; is at risk of having or has had a myocardial infarction, has chronic heart failure, is undergoing coronary intervention, is undergoing percutaneous coronary intervention, is prior to or undergoing or post surgery having a potential to cause cardiac ischemic damage, or is prior to or undergoing or post a cardiac surgery.

8. (canceled)

9. The method of claim 7, wherein the subject with chronic heart failure has chronic or acute myocardial ischemia and reperfusion injury, cardiomyopathy, myocarditis, cardiac hypertrophy, ventricular remodeling, coronary ischemia or congestive heart failure.

10-13. (canceled)

14. The method of claim 4, wherein at least one agent is selected from the group consisting of: a small molecule, a nucleic acid, a nucleic acid analogue, an aptamer, a ribosome, a peptide, a protein, an avimer, an antibody, an siRNA, a miRNA, an shRNA, PNA, pc-PNA or variants or pharmaceutical salts and fragments thereof.

15. The method of claim 4, wherein the agent which activates both an A₁-adenosine receptor and an A_{2A}-adenosine receptor is AMP579 or a derivative thereof.

16. The method of claim 4, wherein the agent which activates both an A₁-adenosine receptor (A₁-AR) and an A_{2A}-adenosine receptor (A_{2A}-AR) is a binary conjugate of at least one agent which activates A₁-AR and at least one agent which activates A_{2A}-AR.

17. A pharmaceutical composition comprising a combination of at least one agent which activates an A₁-adenosine receptor and at least one agent which activates an A_{2A}-adenosine receptor or least one agent which co-activates both an A₁-adenosine receptor A₁-AR) and an A_{2A}-adenosine receptor (A₂-AR and a pharmaceutically acceptable carrier.

18. (canceled)

19. The pharmaceutical composition of claim 17, wherein the pharmaceutical composition results in substantially the same level of biological activation of both the A₁-adenosine receptor and the A_{2A}-adenosine receptor.

20. (canceled)

21. The pharmaceutical composition of claim 17, wherein the agent which co-activates both an A₁-adenosine receptor (A₁-AR) and an A_{2A}-adenosine receptor (A_{2A}-AR) is at least one agent which activates the A₁-adenosine receptor conjugated to at least one agent which activates the A_{2A}-adenosine receptor.

22. The pharmaceutical composition of claim 17, wherein the at least one agent which activates A₁-AR is selected from the group consisting of; AB-MECA, CPA, ADAC, CCPA, CHA, GR79236, S-ENBA, IAB-MECA, R-PIA, ATL146e, CGS-21680, CV1808, NECA, PAPA-APEC, DITC APEC, DPMA, S-PHPNECA, WRC-0470, IB-MECA, 2-CIADO, I-ABA, S-PIA, C1-IB MECA, polyadenylic acid or pharmaceutically acceptable analogues or derivatives or salts thereof.

23. The pharmaceutical composition of claim 17, wherein the at least one agent which activates A_{2A}-AR is selected from the group consisting of; 2-cyclohexylmethylenehydrazinoadenosine, 2-(3-cyclohexenyl)methylenehydrazinoadenosine, 2-isopropylmethylenehydrazinoadenosine, N-ethyl-1′-deoxy-1′-[6-amino-2-[(2-thiazolyl)ethynyl]-9H-purin-9-yl]-β-D-ribofuranuronamide, N-ethyl-1′-deoxy-1′-[6-amino-2-[hexynyl]-9H-purin-9-yl]-β-D-ribofuranuronamide, 2-(1-hexyn-1-yl)adenosine 5′-N-methyluronamide, 5′-chloro-5′-deoxy-2-(1-hexyn-1-yl)adenosine, N6-[2-(3,5-dimethoxyphenyl)-2-(2-methylphenyl)adenosine, 2-(2-phenyl)ethoxyadenosine, 2-[2-(4-methylphenyl)ethoxyl]adenosine, 2-[2-(4-fluorophenyl)ethoxy]adenosine, 2-(2-(2-naphthyl)ethoxy)adenosine, 2-4)-(2-carboxyethyl)phenethylamino-5′-N-ethylcarboxamidoadenosine (CGS-21680), 2-(2-cyclohexyl)ethoxyadenosine, 2-octynyladenosine (YT-146), 2-thiazolylethynyladenosine and 2-phenethylamino-5′-N-ethylcarboxamidoadenosine (CGS-21577) or pharmaceutically acceptable analogues or derivatives or salts thereof.

24-26. (canceled)

27. A pharmaceutical composition comprising an effective amount of AMP 579 or pharmaceutically acceptable analogues or derivatives or salts thereof, and aldose reductase inhibitor.

28. The pharmaceutical composition according to claim 27, wherein the aldose reductase inhibitor is selected from the group consisting of: epalrestat; 3,4-dihydro-2,8-diisopropyl-3-thioxo-2H-1,4-benzoxazine-4-acetic acid; 2,7-difluoro-spiro(9H-fluorene-9,4′-imidazolidine)-2′,5′-dione; 3-[(4-bromo-2-fluorophenyl)methyl]-7-chloro-3,4-dihydro-2,4-dioxo-1(2H)-q-uinazoline acetic acid; 6-fluoro-2,3-dihydro-2′,5′-dioxo-spiro[4H-1-benzopyran-4,4′-imidazolidine]-2-carboxamide; zopolrestat; sorbini; and 1-[(3-bromo-2-benzofuranyl)sulfonyl]-2,4-imidazolidinedione.

29. A method for treating or preventing cardiac dysfunction in a subject having, or at risk of having cardiac dysfunction, the method comprising administering to the subject a pharmaceutical composition comprising an effective amount of an AMP 579 and at least one of an aldose reductase inhibitor or a β-blocker.

30. (canceled)

31. The method of claim 4, wherein the pharmaceutical composition comprises a β-blocker or an aldose reductase inhibitor or a β-blocker and an aldose reductase inhibitor.

32. (canceled)

33. The pharmaceutical composition of claim 17, wherein the pharmaceutical composition optionally comprises a β-blocker or an aldose reductase inhibitor or a β-blocker and an aldose reductase inhibitor.

34. (canceled)

35. The method of claim 4, wherein the wherein the pharmaceutical composition results in a level of biological activation of the A₁-adenosine receptor is within about 10% of the level of biological activation of the A_{2A}-adenosine receptor, or wherein the at least one agent that activates the A₁-adenosine receptor has a lower Ki as compared to Ki of at least one agent which activates the A_{2A}-adenosine receptor.

36. The method of claim 4, wherein the pharmaceutical composition comprising an effective amount of a combination of at least one agent which activates an A₁-adenosine receptor (A₁-AR) and at least one agent which activates an A_{2A}-adenosine receptor (A_{2A}-AR), comprises at least a 1.5 fold higher amount of the at least one agent which activates the A₁-adenosine receptor as compared to the amount of the at least one agent which activates the A_{2A}-adenosine receptor activation.
